(19) United States
(12) Patent Application Publication
(10) Pub. No.: US 2019/0225682 A1
(43) Pub. Date: Jul. 25, 2019

(54) METHOD OF TREATING LOCALIZED FIBROTIC DISORDERS USING AN IL-33/TNF BISPECIFIC ANTIBODY

(71) Applicant: 180 Therapeutics LP, Cambridge, MA (US)

(72) Inventors: Jagdeep Nanchahal, Headington (GB); Glenn R. Larsen, Sudbury, MA (US); Marc Feldmann, London (GB)

(21) Appl. No.: 16/328,979

(22) PCT Filed: Aug. 31, 2017
(86) PCT No.: PCT/US17/49691

§ 371 Date: Feb. 27, 2019

Related U.S. Application Data
(60) Provisional application No. 62/383,246, filed on Sep. 2, 2016.

(51) Int. Cl. C07K 16/24; C12N 15/115; A61P 17/00; A61P 19/02; A61P 17/02; A61P 15/00; A61P 19/04

(52) U.S. Cl. CPC C07K 16/244; C07K 16/241; C12N 15/115; A61P 17/00; A61P 19/02; A61K 2039/505; A61P 15/00; A61P 19/04; C07K 2317/31; C07K 2317/76; C12N 2310/16; A61P 17/02

(57) ABSTRACT

The subject invention provides a method of treating a patient suffering from a localized fibrotic condition which comprises administering to the patient an amount of an IL-33 antagonist effective to treat the patient. The invention also provides a method of treating a patient suffering from a localized fibrotic condition which comprises administering to the patient an amount of a bispecific antibody comprising an IL-33 antigen binding domain of which (i) binds to and inhibits activation of, an IL-33 receptor, or (ii) specifically binds to IL-33 and inhibits IL-33 from binding to the IL-33 receptor, and a TNF antigen binding domain of which (i) binds to and inhibits activation of, a TNF receptor, or (ii) specifically binds to TNF and inhibits TNF from binding to the TNF receptor, wherein the bispecific antibody is effective to treat the patient.

This application claims priority of U.S. Provisional Application No. 62/383,246, filed Sep. 2, 2016, the entire contents of which are hereby incorporated by reference herein.

Throughout this application various publications are referenced, most typically by the last name of the first author and the year of publication. Full citations for these publications are set forth in a section entitled References immediately preceding the claims. The disclosures of all referenced publications in their entireties are hereby incorporated by reference into this application in order to more fully describe the state of the art to which the invention relates.

### BACKGROUND OF INVENTION

### Fibrosis

Fibrosis is characterized by the excess accumulation of extracellular matrix (ECM) components including collagen. Wynn (2008) J. Pathol. 214:199-210; Sivakumar & Das (2008) Inflamm. Res. 57:410-418. Fibrosis is thought to be a consequence of chronic tissue irritation or damage. Wynn (2008) J. Pathol. 214:199-210; Friedman (2008) Gastroenterology 134:1655-1669; Trojanowska & Varga (2007) Curr. Opin. Rheumatol. 19:568-573; Selman & Pardo (2006) Proc. Am. Thorac. Soc. 3:364-372. The progressive replacement of parenchymal tissues with ECM is observed in fibrotic diseases such as systemic sclerosis, idiopathic pulmonary fibrosis and liver fibrosis, leading to impaired organ function. Fibrosis is estimated to contribute to nearly 45% of deaths in the developed world. However, the cellular and molecular factors that sustain the fibrotic cascade remain poorly understood (U.S. Patent Application Publication No. 20140212412).

Fibrotic conditions occur when processes that normally contribute to wound healing go awry, resulting in extra scar tissue that can be harmful. Fibrotic conditions can affect single organs or tissues.

### Dupuytren's Disease

Dupuytren's disease, also known as palmar fibromatosis or in its established disease stage Dupuytren's contracture, is a disease associated with the buildup of extracellular matrix materials such as collagen on the connective tissue of the hand (the palmar fascia) causing it to thicken and shorten with the result that the fingers curl into the palm.

Dupuytren's disease is a common fibrotic disorder (Hindocha, 2009). The mean age of treatment for the disease is 63 years (Chen, 2011), with onset approximately 10 years earlier. It exhibits a strong hereditary basis (Hurst, 2009). Dupuytren's disease causes the fingers to curl irreversibly into the palm, leading to significant impairment of hand function.

There is no approved treatment for early disease. Once patients have established deformities, the mainstay of treatment is surgical excision (fasciectomy) of the diseased tissue or cords (Davis, 2013). Patients with advanced disease are treated by surgical excision of diseased tissues. Surgery is recommended when patients develop flexion deformities of the digits of 30 degrees or more of the finger joints and suffer impaired hand function (Rayan, 2007). Between 10-12% of patients develop recurrence over 3 years following surgery (Ullah, 2009; van Rijssen, 2012) and are treated with more extensive surgery that involves excision of the diseased tissue and the overlying skin (dermofasciectomy). Post-operatively, patients require 3-6 months of hand therapy and splintage (Hughes, 2003; Larson, 2008). Complications occur in about 20% of surgical patients (Bulstrode, 2005; Crean, 2011).

Alternative, less invasive techniques have been developed to disrupt the cords of diseased tissue with either a needle (Beaudreuil, 2012) or collagenase digestion (Hurst, 2009). However, recurrence rates are high, affecting 70% of patients treated with percutaneous needle fasciotomy (van Rijssen, 2012) and 35% of those treated with collagenase (Peimer, 2013) at 3 years. The complication rate is 20% following needle fasciotomy (Crean, 2011) and over 70% after collagenase injection (Hurst, 2009).

In the United Kingdom, the vast majority of patients with established disease and finger contractures are treated surgically (Davis, 2013). Over 90% of the 12,900 patients who have surgery for Dupuytren's disease per annum in the United Kingdom undergo fasciectomy. Recurrence rates are of the order of 12% within 3 years of fasciectomy and the costs for dermofasciectomy for recurrent disease are much higher (Ullah, 2009). Neither surgical fasciectomy nor collagenase injection was found to be an effective use of health care resources (Chen, 2011).

Intralesional steroid injection and radiotherapy are two additional possible treatments for Dupuytren's disease (Ball, 2016). Intralesional steroid injection has been proposed based on a retrospective study of 63 patients with early Dupuytren's disease treated with steroid injection into the nodules at 6 week intervals (Ketchum, 2000). However, this treatment has found limited acceptance. Radiotherapy has also been used although 20-30% of patients developed long term adverse effects, including dry skin, increased desquamation, skin atrophy, telangiectasia, erythema, altered heat and pain sensation (Seegenschmiedt, 2001; Pohl, 2002; Betz, 2010). Based on the published data The National Institute for Health and Care Excellence (NICE) does not recommend radiotherapy for Dupuytren's disease (NICE, 2010).

Therefore, there is a need to develop an effective therapy to prevent progression of early Dupuytren's disease while avoiding the necessity for invasive procedures. Also, there is a need to prevent the development of recurrent disease following surgery, needle fasciotomy, or collagenase injection in patients with established finger contractures.

### Combination Therapy

The administration of two drugs to treat a given condition, such as a localized fibrotic disorder, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry, 1999). In one example, combined administration of glatiramer acetate (GA) and interferon (IFN) has been experimentally shown to abrogate the clinical effectiveness of either therapy (Brod 2000). In another experiment, it was reported that the addition of prednisone in combination therapy with IFN-β antagonized its up-regulator effect. Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry, 1999). The interaction may also heighten or lessen the side effects of each drug. Hence, upon administration of two drugs to treat a disease, it is unpredictable what change will occur in the negative side profile of each drug. In one example, the combination of natalizumab and interferon β-1a was observed to increase the risk of unanticipated side effects. (Vollmer, 2008; Rudick 2006; Kleinschmidt-DeMasters, 2005; Langer-Gould 2005)

Additionally, it is difficult to accurately predict when the effects of the interaction between the two drugs will become manifest. For example, metabolic interactions between drugs may become apparent upon the initial administration of the second drug, after the two drugs have reached a steady-state concentration or upon discontinuation of one of the drugs (Guidance for Industry, 1999).

Therefore, the state of the art at the time of filing is that the effects of a combination therapy of two drugs, in particular an IL-33 antagonist together with either a TNF antagonist or a TNF soluble receptor, or a bispecific antibody directed to both IL-33 and TNF, cannot be predicted with any reasonable certainty.

### Bispecific Antibodies

By unifying two antigen binding sites of different specificity into a single construct, bispecific antibodies have the ability to bring together two discrete different antigens with specificity and therefore have great potential as therapeutic agents.

The major promise of bispecific antibodies is the enhancement of antibody efficacy by combining up to two activities into one molecule thus allowing the neutralization of biologic activity of each of the two ligands simultaneously by the binding of one mAb, the inhibition of two receptors by one mAb, the crosslinking of two receptors on one cell or the redirecting of cytotoxic immune cells. Another benefit of bispecific antibodies is the avoidance of some of the dual development effort and cost.

There are number of approaches for obtaining such bispecific antibodies. Bispecific antibodies were originally made by fusing two hybridomas, each capable of producing a different immunoglobulin. The resulting hybrid-hybridoma, or quadroma, was capable of producing antibodies bearing the antigen specificity of the first parent hybridoma as well as that of the second parent hybridoma (Milstein et al. (1983), Nature 305:537). However, the antibodies resulting from quadromas often exhibited undesired properties due to the presence of an Fc antibody portion.

Largely due to such difficulties, attempts later focused on creating antibody constructs resulting from joining two scFv antibody fragments while omitting the Fc portion present in full immunoglobulins. Each scFv unit in such constructs was made up of one variable domain from each of the heavy (VH) and light (VL) antibody chains, joined with one another via a synthetic polypeptide linker, the latter often being genetically engineered so as to be minimally immunogenic while remaining maximally resistant to proteolysis. The resulting bispecific single chain antibody is therefore a species containing two VH/VL pairs of different specificity on a single polypeptide chain, wherein the VH and VL domains in a respective scFv unit are separated by a polypeptide linker long enough to allow intramolecular association between these two domains, and wherein the thusly formed scFv units are contiguously tethered to one another through a polypeptide spacer kept short enough to prevent unwanted association between, for example, the VH domain of one scFv unit and the VL of the other scFv unit.

Bispecific single chain antibodies of the general form described above have the advantage that the nucleotide sequence encoding the four V-domains, two linkers and one spacer can be incorporated into a suitable host expression organism under the control of a single promoter or multiple promoters for different polypeptide encoding units. This increases the flexibility with which these constructs can be designed as well as the degree of experimenter control during their production.

Remarkable experimental results have been obtained using such bispecific single chain antibodies designed for the treatment of malignancies (Mack, J. Immunol. (1997), 158:3965-70; Mack, PNAS (1995), 92:7021-5; Kufer, Cancer Immunol. Immunother. (1997), 45:193-7; Loffler, Blood (2000), 95:2098-103) and non-malignant diseases (Bruhl, J. Immunol. (2001), 166:2420-6). In such bispecific single chain antibodies, one scFv unit is capable of activating cytotoxic cells, for example cytotoxic T cells, within the immune system by specifically binding to an antigen on the cytotoxic cells, while the other scFv unit specifically binds an antigen on a malignant cell intended for destruction. In this way, such bispecific single chain antibodies have been shown to activate and redirect the immune system's cytotoxic potential to the destruction of pathological, especially malignant cells. In the absence of such a bispecific single chain antibody construct, malignant cells would otherwise proliferate uninhibited.

In the event that a bispecific antibody is intended for therapeutic use, it is desirable to produce high amounts of this antibody solubly and in the desired functional form. The production of functionally active antibody becomes especially critical when producing bispecific antibodies of which one portion is able to activate and recruit the cytotoxic potential of human immune effector cells. For example, a produced antibody devoid of functional activity will not lead to the desired activation of human immune effector cells, while a bispecific antibody which is functionally active, albeit not in the desired manner, as for example may be the case when the bispecific antibody is produced in a heterogeneous form containing multiple isomers, may activate and recruit the cytotoxic potential of human immune effector cells in unforeseeable and/or unintended manners.

### SUMMARY OF THE INVENTION

The subject invention provides a method of treating a patient suffering from a localized fibrotic condition which comprises administering to the patient an amount of an IL-33 antagonist effective to treat the patient.

The subject invention further provides a method of treating a patient suffering from a localized fibrotic condition which comprises administering to the patient an amount of a soluble TNF receptor 2 (TNFR2) antagonist effective to treat the patient.

The subject invention further provides a method of treating a patient suffering from a localized fibrotic condition which comprises administering to the patient an amount of a soluble TNF receptor 1 (TNFR1) antagonist effective to treat the patient.

The invention also provides a method of treating a patient suffering from a localized fibrotic condition which comprises administering to the patient an amount of a bispecific antibody comprising
a) a IL-33 antigen binding domain of which (i) binds to and inhibits activation of, an IL-33 receptor, or (ii) specifically binds to IL-33 and inhibits IL-33 from binding to the IL-33 receptor, and
b) a TNF antigen binding domain of which (i) binds to and inhibits activation of, a TNF receptor, or (ii) specifically binds to TNF and inhibits TNF from binding to the TNF receptor,
   effective to treat the patient.

### BRIEF DESCRIPTION OF THE FIGURES

FIG. 1: Immune cells are present in Dupuytren's myofibroblast-rich tissue and release pro-inflammatory cytokines. (A) Flow cytometric analysis of cells isolated from freshly disaggregated Dupuytren's tissue. Intracellular α-SMA-positive (myofibroblasts; mean±SD: 87±6.1%), cell surface CD68-positive/CD163-negative (classically activated M1 macrophages; mean±SD: 4.8±2.2%), CD68-positive/CD163-positive (alternatively activated M2 macrophages; mean±SD: 1.8±1.0%) and CD117-positive (mast cells; mean±SD: 2.8±2.6% cells were quantified.) (B) Serial histological sections of Dupuytren's tissue stained for α-SMA+ (myofibroblasts), CD68+(monocytes) and chymase+ (mast cells) (Scale bar, 100 μm.)

FIG. 2: TNF selectively induces IL-33 mRNA expression in palmar dermal fibroblasts. 0.1 ng/ml rhTNF stimulation for 24 h selectively induced IL-33 mRNA expression in dermal palmar fibroblasts (PF-D) at hours. rhTNF did not have any effect on non-palmar dermal fibroblasts from Dupuytren's patients (NPF-D), or palmar dermal fibroblasts from normal individuals without Dupuytren's disease (PF-N). n=5 patients for all cell types. **P<0.001

FIG. 3: Myofibroblasts from patients with Dupuytren's disease (MF-D) respond to neutralizing anti-IL-33 in a dose-dependent manner. (A) Anti-IL-33 downregulates relative COL1A1 and α-SMA mRNA expression; (B) Anti-IL-33 downregulates the relative expression of TNF receptor 1 (TNFR1) and TNF receptor 2 (TNFR2) (C) Anti-IL-33 downregulates relative expression of mRNA of IL-33 and its cell surface receptor ST2L. All values were normalized to fold change compared to untreated MF-D. n=3 for 0.04 ug/ml and 4 ug/ml anti-IL-33 and n=6 for 0.4 ug/ml anti-IL-33. IgG isotype control for anti-IL-33 showed no effect in the relative expression of the genes at the corresponding doses tested. Data expressed as mean±SEM. *P<0.05, **P<0.01, ***P<0.001, ****P<0.0001. Methods: 1×10⁶ cells were cultured in monolayer and treated with rhTNF (300-01A, Peprotech), neutralizing anti-TNF (MAB2101, R&D), neutralizing anti-TNF receptor 1 (MAB625, R&D), neutralizing anti-TNF receptor 2 (MAB726, R&D), anti-TNF/TNF receptors isotype control (MAB002, R&D), neutralizing anti-IL-33 (500-P261, Peprotech) or isotype control (500-P00, Peprotech). The total RNA was extracted from each sample using a QIAshredder, followed by QIAamp RNeasy Mini Kit (74104, Qiagen) with on-column RNase-Free DNase set (79254, Qiagen) according to the manufacturer's instructions. RNA was eluted in 30 μl RNase-free water provided and quantified using a NanoDrop ND-1000 spectrophotometer (NanoDrop Technologies), ensuring a 260/230 and 280/260 ratios >2.0. For real-time reverse transcription PCR, Inventoried TaqMan® Gene expression Assays were used for α-SMA (Hs00426835-g1), COL1A1 (Hs00164004-m1), TNFR1 (Hs01042313-m1) and TNFR2 (Hs00961749-m1), IL-33(Hs00369211_m1) and ST2 (Hs00545033_m1) (Applied Biosystems) with Reverse Transcriptase qPCR™ Mastermix No ROX (RT-QPRT-032XNR, Eurogentec). A total of 10 μl of reaction mixture containing 2 μl of RNA at 50 ng/ml, 5 μl of 2× buffer, 0.5 μl Taqman probe, 0.05 μl of Reverse Transcriptase enzyme with RNase inhibitors and 2.45 μl RNase free water were added to each well of a 384 well plate. Samples were run on the ABI VAii 7™ Real-Time PCR System (Applied Biosystems). Expression was normalized to GAPDH (Hs02758991-g1, Applied Biosystems) and compared to the level of gene expression in baseline respective cell types, which were assigned the value of 1 using delta delta CT analysis performed with SDS software (Applied Biosystems).

FIG. 4A-4C: Immune cells are present in Dupuytren's nodules and secrete cytokines.

FIG. 4A: Characterization of cells in Dupuytren's nodules by FACS. The majority of the cells present are myofibroblasts, there are significant numbers of CD45+ immune cells, with macrophages, including classically activated (M1) and alternatively activated (M2) phenotypes, T cells and mast cells.

FIG. 4B: Immunostaining of Dupuytren's nodules. The majority of the cells are α-SMA positive myofibroblasts with interspersed CD68+ macrophages and tryptase positive mast cells.

FIG. 4C: Chemokines secreted by freshly disaggregated cells from Dupuytren's nodules. Chemokine levels were detected by electrochemiluminescence assays in the supernatants of freshly disaggregated Dupuytren's nodular cells after 24 hours. n=40 patient samples. CCL2 and CXCL10 are known chemoattractants for macrophages and CXCL8 (IL-8), CCL26 and CXCL10 for mast cells.

| | CXCL8 | CCL2 | CCL26 | CXCL10 | CCL17 | CCL3 |
|---|---|---|---|---|---|---|
| Mean | 3376 | 955.0 | 1056 | 238.8 | 57.37 | 57.61 |
| (pg/ml) | | | | | | |
| Std. | 2069 | 993.1 | 375.2 | 115.5 | 33.08 | 46.44 |
| Deviation | | | | | | |
| Std. Error of | 354.8 | 167.9 | 73.58 | 21.10 | 6.366 | 9.683 |
| Mean | | | | | | |

| | CCL11 | CCL4 | CCL13 | CCL22 | CCL8 | SCF |
|---|---|---|---|---|---|---|
| Mean (pg/ml) | 45.15 | 49.05 | 34.40 | 25.88 | 6.722 | 10.85 |
| Std. Deviation | 26.64 | 58.88 | 14.39 | 11.56 | 5.592 | 9.004 |
| Std. Error of | 4.638 | 12.85 | 2.628 | 2.313 | 1.318 | 1.592 |
| Mean | | | | | | |

FIGS. 5A-5G: Dupuytren's disease is a localized inflammatory disorder characterized by the secretion of cytokines, including TNF, which leads to increased expression of TNFR2 in palmar fibroblasts and myofibroblasts from patients with Dupuytren's disease.

FIG. 5A: A range of cytokines are secreted, including TNF and IL-33. Cytokines released by freshly isolated nodular cells in monolayer culture for 24 hours were measured using electrochemiluminescence. N=20 samples for TGFβ1 and 40 for all other cytokines.

| | TGF-β1 | IL-6 | TNF | IL-1β | IFN-γ | GM-CSF |
|---|---|---|---|---|---|---|
| Mean | 306.2 | 4333 | 55.75 | 12.11 | 4.786 | 48.79 |
| (pg/ml) | | | | | | |
| Std. | 231.6 | 3465 | 37.49 | 10.17 | 5.424 | 34.58 |
| Deviation | | | | | | |
| Std. Error of | 48.28 | 534.7 | 5.784 | 1.570 | 0.8369 | 5.336 |
| Mean | | | | | | |

| | IL-33 | M-CSF | IL-13 | IL-17A | IL-10 | IL-4 |
|---|---|---|---|---|---|---|
| Mean (pg/ml) | 13.59 | 2.617 | 1.915 | 0.08417 | 7.772 | 0.5413 |
| Std. Deviation | 20.70 | 2.496 | 0.6281 | 0.03632 | 9.713 | 1.144 |
| Std. Error of | 2.988 | 0.2942 | 0.07205 | 0.006524 | 1.499 | 0.1832 |
| Mean | | | | | | |

FIG. 5B: Cytokine levels do not depend on cell concentration. TNF secreted by varying numbers of freshly disaggregated cells from Dupuytren's nodules incubated for 24 hours in 4 ml of culture medium (DMEM) and 5% fetal bovine serum. The levels of TNF were determined by ELISA.

FIG. 5C: Cytokines in the plasma of patients with Dupuytren's disease compared with those secreted by freshly disaggregated nodular cells. Plasma levels of TNF, IL-1β, IL-6 and IL-8 were much lower in the systemic circulation.

FIG. 5D: Characterization of cells in Dupuytren's nodules secreting TNF. The cells expressing TNF by FACS included macrophages, both classically and alternatively activated mast cells and T cells.

FIG. 5E: Palmar dermal fibroblasts but not non-palmar dermal fibroblasts from the same individuals with Dupuytren's disease show increased expression of TNFR2 but not TNFR1 on treatment with TNF. Dupuytren's disease only occurs in the palm of genetically susceptible individuals. Exposure to physiologically relevant levels (0.1 ng/ml) of TNF of the palmar dermal fibroblasts from these patients resulted in increased expression of the inducible TNFR2 whilst expression of TNFR1 is reduced in these cells at both mRNA and protein level when exposed.

FIG. 5F: Immunostaining of TNFR1 and TNFR2 in Dupuytren's nodules. The majority of the cells in Dupuytren's nodules express both TNFR1 and TNFR2.

FIG. 5G: Palmar dermal fibroblasts and myofibroblasts show increased expression of TNFR2 but not TNFR1 on treatment with TNF. Non-palmar dermal fibroblasts from the same individuals with Dupuytren's disease show decreased expression of TNFR2. Quantification of immunofluorescent staining of matched cells from 3 donors. 20 cells were assessed from each patient.

FIGS. 6A-6E: IL-33 produced by myofibroblasts acts on mast cells and alternatively activated (M2) macrophages leading to increased TNF expression.

FIG. 6A: Myofibroblasts from Dupuytren's nodules express IL-33. The majority of the cells expressing IL-33 by FACS are myofibroblasts.

FIG. 6B: Immunofluorescence staining of ST2 and IL-33 freshly isolated myofibroblasts from Dupuytren's nodules. ST2 labels the cell surface whilst the IL-33 is seen both within the nucleus and cytoplasm.

FIG. 6C: Freshly isolated mast cells and macrophages from Dupuytren's nodules express ST2, the receptor for IL-33. Immunofluorescence co-staining.

FIG. 6D: Mast cell lines show increased TNF secretion on exposure to IL-33 in a dose-dependent manner.

FIG. 6E: Only alternatively activated macrophages (M2) derived from human monocytes and pre-treated with TNF show increased secretion of TNF on exposure to IL-33 in a dose-dependent manner.

FIGS. 7A-7C: Palmar fibroblasts but not non-palmar fibroblasts from patients with Dupuytren's disease differentiate into myofibroblasts and show increased expression of IL-33 and ST2 on exposure to TNF.

FIG. 7A: Only palmar fibroblast differentiate into myofibroblasts as evidenced by increased α-SMA at mRNA and protein levels and increased COL1A1 mRNA expression on treatment with 0.1 ng/ml TNF.

FIG. 7B: Only palmar fibroblast show increased expression of IL-33 and ST2 at both mRNA and protein levels whilst non-palmar fibroblasts show reduced expression of ST2 on exposure to TNF.

FIG. 7C: Palmar fibroblasts show increased expression of nuclear and cytoplasmic IL-33 and ST2 on treatment with TNF. Quantification of immunofluorescent staining of matched cells from 3 donors. 20 cells of each type were assessed from every patient.

FIGS. 8A-8D: Inhibition of TNF, TNFR2 or IL-33 down regulates the myofibroblast phenotype, with a combination of TNFR2 and IL-33 being most effective.

FIG. 8A: Anti-IL-33 down regulates the expression of α-SMA and ST2 at both the mRNA and protein level and COL1A1 at the mRNA level in myofibroblasts from patients with Dupuytren's disease in a dose-dependent manner. Data from non-responders not shown.

FIG. 8B: Only inhibition of TNF or TNFR2 but not TNFR1 downregulates the expression of α-SMA, COL1-A1, IL-33 and ST2 at mRNA level and 11-33 and ST2 also at protein level in myofibroblasts from responsive myofibroblasts from patients with Dupuytren's disease. Data from non-responders not shown.

FIG. 8C: Venn diagram showing the relative efficacy of TNF or IL-33 or TNFR2 inhibition. α-SMA was down regulated in myofibroblasts 6 of 11 patient samples (55%) by anti-TNF, 8 of 11 patient samples (73%) by anti-IL-33 and in 8 of 11 samples by anti-TNFR2. Therefore, combined anti-TNF and anti-IL-33 would be effective in 9 out of 11 patient samples (82%) and anti-TNFR2 and anti-IL-33 in 11 of 11 samples (100%).

FIG. 8D: Inhibition of expression of TNFR2, ST2 and most effectively TNFR2+ST2 down regulates myofibroblast phenotype

FIG. 9: Proposed mechanism of action of IL-33. TNF secreted by resident immune cells, including macrophages and mast cells, converts precursor cells into myofibroblasts. As the cells differentiate into myofibroblasts, they secrete IL-33. This in turn acts on the immune cells, leading to further TNF production through a positive feedback loop, resulting in chronic localized inflammation and a fibrotic response.

FIG. 10: Proposed mechanism of action of IL-33. TNF secreted by resident immune cells, including macrophages and mast cells, converts precursor cells into myofibroblasts. As the cells become myofibroblasts, they secrete IL-33, which acts on the immune cells, leading to further TNF production, driving a positive feedback loop and a chronic fibrotic response. The IL-33 also acts on the myofibroblasts via ST2 and further enhances IL-33 expression.

FIG. 11: A schematic representation of Dual Variable Domain (DVD)-Ig constructs and shows the strategy for generation of a DVD-Ig from two parent antibodies.

In some of the following figures, letters are occasionally assigned to a part of the depicted antibody. This is used to show where that part of the depicted antibody is in the next depicted antibody. The letters assigned in each figure are not related to letters assigned in other figures.

FIG. 12: Fab in Tandem bispecific antibody.

FIG. 13: Alternative formats for bispecific antibodies and other bispecific immunotherapeutics subdivided into five major classes: BsIgG, appended IgG, BsAb fragments, bispecific fusion proteins and BsAb conjugates. Heavy chains are shown in dark shades and corresponding light chains are in light shades. Connecting peptide linkers and engineered disulfide bonds are shown by thin lines. Approximate molecular weights are shown assuming ˜12.5 kDa per immunoglobulin domain.

FIG. 14: Schematic representation of targeted hetero-association of functional domains by fusion to a segmented tertiary structure.

FIG. 15: Asymmetric IgG-Like bispecific antibodies

FIG. 16: Symmetric IgG-Like bispecific antibodies

FIG. 17: IgG Fusions bispecific antibodies

FIG. 18: Fc Fusions bispecific antibodies

FIG. 19: Fab Fusions bispecific antibodies

FIG. 20: ScFv- and Diabody-based bispecific antibodies

FIG. 21: IgG/Non-IgG Fusions bispecific antibodies

FIG. 22: Heterodimeric IgG bispecifics (including Quadroma Triomab, Knobs-into-holes In Vitro assembly, Common LC, CrossMab^{CH1-CL}, (SEED) body, and LUZ-Y)

FIG. 23: Bispecific Abs from fragments

FIG. 24: This figure shows A) VH and B) VL sequences of anti-ST2L antibodies derived from phage display libraries and after subsequent affinity-maturation campaigns.

FIG. 25: This figure shows VH and VL regions and sequences of heavy chain CDRs of anti-ST2L antibody STLM208 VH ST2H257 HCDR3 variants.

FIG. 26: (A-H) Dual targeting strategies utilizing bispecific antibodies: (A) neutralization of two receptor-activating ligands, (B) neutralization of two receptors, (C) neutralization of a receptor and a ligand, (D) activation of two receptors, (E) activation of a receptor and inactivation of another receptor, (F) activation of a receptor and inactivation of a ligand, (G) blockage of two epitopes of one receptor, (H) blockage of two epitopes of one ligand. (I-O) Dual retargeting strategies utilizing bispecific antibodies: (I) binding to two receptors and Fc-mediated ADCC or CDC, (K) retargeting of cytotoxic effector cells with a trispecific antibody, (L) targeting of a bispecific toxin (immunotoxin) or a bispecific antibody-drug conjugate (ADC) to two receptors, (M) targeting of a bispecific cytokine (immunocytokine) to two receptors, (N) targeting of an enzyme to two receptors, (O) targeting of a drug-loaded nanoparticle/liposome to two receptors. Strategies are exemplified with bispecific IgG and Fab molecules, respectively.

FIG. 27: Bispecific antibody formats. Variable heavy chain domains (VH) are shown by a dark shade and identified by the letters b and d, variable light chain domains (VL) are shown by a light shade and identified by the letters a and c. The letters a and b and c and d indicate different specificities. Antibody constant domains are shown in white boxes and fusion proteins in white circles.

### DETAILED DESCRIPTION OF THE INVENTION

### Terms

As used herein, and unless stated otherwise, each of the following terms shall have the definition set forth below.

As used herein, including the appended claims, the singular forms of words such as “a,” “an,” and “the,” include their corresponding plural references unless the context clearly dictates otherwise.

As used herein, “an effective amount” is an amount effective to yield a desired therapeutic response without undue adverse side effects (such as toxicity, irritation, or allergic response) commensurate with a reasonable benefit/risk ratio. The effective amount will vary with such factors as the particular condition being treated, the physical condition of the patient, the duration of the treatment, the nature of concurrent therapy (if any), the specific formulations employed and the structure of the compound being administered.

As used herein, “about” in the context of a numerical value or range means±10% of the numerical value or range recited or claimed.

As used herein, to “treat” or “treating” means inducing inhibition, regression, or stasis of a disorder and/or disease. As used herein, “inhibition” of disease progression or disease complication in a subject means preventing, reducing or reversing the disease progression or disease complication in the subject.

The term “antibody”, as used herein, means any immunoglobulin (Ig) molecule comprised of four polypeptide chains, two heavy (H) chains and two light (L) chains in which one heavy chain and one light chain can bind to an antigen and the second heavy chain and the second light chain can bind to a second antigen which may be the same or different.

In a full-length antibody, each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are generally conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG1, IgG2, IgG 3, IgG4, IgA1 and IgA2) or subclass.

A “bispecific antibody” as used herein means any antibody with two binding domains that binds, and preferably neutralize biological function of, two distinct antigens in which the determinants of each of the antigens are different, i.e. two different antigens with different antigenic determinants.

The term “Fc region” defines the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain and a CH3 domain, and optionally comprises a CH4 domain. Replacements of amino acid residues in the Fc portion to alter antibody effector function are known in the art (Winter, et al. U.S. Pat. Nos. 5,648,260 and 5,624,821). The Fc portion of an antibody mediates several important effector functions e.g., cytokine induction, ADCC, phagocytosis, complement dependent cytotoxicity (CDC) and half-life/clearance rate of antibody and antigen-antibody complexes. In some cases these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to Fc.gamma.Rs and complement C1q, respectively. Neonatal Fc receptors (FcRn) are the critical components determining the circulating half-life of antibodies. Additionally, at least one amino acid residue may be replaced in the constant region of the antibody, for example the Fc region of the antibody, such that effector functions of the antibody are altered. The dimerization of two identical heavy chains of an immunoglobulin is mediated by the dimerization of CH3 domains and is stabilized by the disulfide bonds within the hinge region (Huber et al. Nature; 264: 415-20; Thies et al 1999 J Mol Biol; 293: 67-79.). Mutation of cysteine residues within the hinge regions to prevent heavy chain-heavy chain disulfide bonds will destabilize dimerization of CH3 domains. Residues responsible for CH3 dimerization have been identified (Dall'Acqua 1998 Biochemistry 37: 9266-73.). Therefore, it is possible to generate a monovalent half-Ig. Such monovalent half Ig molecules have been found in nature for both IgG and IgA subclasses (Seligman 1978 Ann Immunol 129: 855-70; Biewenga et al 1983 Clin Exp Immunol 51: 395-400). The stoichiometry of FcRn: Ig Fc region has been determined to be 2:1 (West et al 0.2000 Biochemistry 39: 9698-708), and half Fc is sufficient for mediating FcRn binding (Kim et al 1994 Eur J Immunol; 24: 542-548.). Mutations to disrupt the dimerization of CH3 domain may not have greater adverse effect on its FcRn binding as the residues important for CH3 dimerization are located on the inner interface of CH3 b sheet structure, whereas the region responsible for FcRn binding is located on the outside interface of CH2-CH3 domains. However, the half Ig molecule may have certain advantage in tissue penetration due to its smaller size than that of a regular antibody. At least one amino acid residue may be replaced in the constant region of the binding protein of the invention, for example the Fc region, such that the dimerization of the heavy chains is disrupted, resulting in half DVD Ig molecules. The anti-inflammatory activity of IgG is dependent on sialylation of the N-linked glycan of the IgG Fc fragment. The precise glycan requirements for anti-inflammatory activity has been determined, such that an appropriate IgG1 Fc fragment can be created, thereby generating a fully recombinant, sialylated IgG1 Fc with greatly enhanced potency (Anthony, R. M., et al. (2008) Science 320:373-376).

The term “vector”, as used herein, means a DNA or RNA molecule such as a plasmid, virus or other vehicle, which may contain one or more heterologous or recombinant DNA sequences and is designed for transfer between different host cells. The terms “AAV expression vector” and “AAV vector” refer to any adeno-associated virus (AAV) vector effective to incorporate and express heterologous DNA sequences in a cell. In connection with this invention any suitable AAV vector can be employed that is effective for introduction of nucleic acids into cells such that protein or polypeptide expression in the cell results. Various cells effective for expression, e.g., mammalian cells such as Chinese Hamster Ovary (CHO) cells, insect cells and eukaryotic cells such as yeast cells are useful in practicing the invention.

The term “antigen-binding portion” of an antibody refers to one or more portions of an antibody that possess the ability to bind specifically to an antigen and preferably neutralize biological function of the antigen and enhances the clearance of the antigen from the body. It has been shown that the antigen-binding function of an antibody can be performed by certain fragments of a full-length antibody such as a bispecific antibody. Examples of “antigen-binding portions” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward (1989) Nature 341:544-546; PCT Publication No. WO 90/05144 A1), which comprises a single variable domain; and (vi) an isolated complementarity determining region (CDR). Further, although the two domains of the Fv fragment, VL and VH, are encoded by separate genes, they can be joined, using recombinant methods, by a synthetic linker that enables them to be expressed as a single protein chain in which the VL and VH regions pair to form monovalent molecules (also known as single chain Fv (scFv); see e.g., Bird et al. (1988) Science 242: 423-426; and Huston et al. (1988) Proc. Natl. Acad. Sci. USA 85: 5879-5883). Such single chain antibodies are also intended to be encompassed within the term “antigen-binding portion” of an antibody. Other forms of single chain antibodies are encompassed, such as diabodies which are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2: 1121-1123). Such antibody binding portions are known in the art (Kontermann and Dubel eds., Antibody Engineering (2001) Springer-Verlag. New York. p. 790 (ISBN 3-540-41354-5). In addition, single chain antibodies include “linear antibodies” comprising a pair of tandem Fv segments (VH-CH1-VH-CH1) which, together with complementary light chain polypeptides, form a pair of antigen binding regions (Zapata et al. (1995) Protein Eng. 8(10):1057-1062; and U.S. Pat. No. 5,641,870).

The term “linker” is used to denote polypeptides comprising two or more amino acid residues joined by peptide bonds and are used to link one or more antigen binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). Exemplary linkers include, but are not limited to, AKTTPKLEEGEFSEAR; AKTTPKLEEGEFSEARV; AKTTPKLGG; SAKTTPKLGG; SAKTTP; RADAAP; RADAAPTVS; RADAAAAGGPGS; RADAAAA(G4S)4; SAKTTPKLEEGEFSEARV; ADAAP; ADAAPTVSIFPP; TVAAP; TVAAPSVFIFPP; QPKAAP; QPKAAPSVTLFPP; AKTTPP; AKTTPPSVTPLAP; AKTTAP; AKTTAPSVYPLAP; ASTKGP; ASTKGPSVFPLAP, GGGGSGGGGSGGGGS; GENKVEYAPALMALS; GPAKELTPLKEAKVS; GHEAAAVMQVQYPAS, TVAAPSVFIFPPTVAAPSVFIFPP; and ASTKGPSVFPLAPASTKGPSVFPLAP.

An “immunoglobulin constant domain” refers to a heavy or light chain constant domain. Human IgG heavy chain and light chain constant domain amino acid sequences are known in the art.

“Specific” and “specificity” in the context of an interaction between members of a specific binding pair (e.g., an antigen (or fragment thereof) and an antibody (or antigenically reactive fragment thereof)) refer to the selective reactivity of the interaction. The phrase “specifically binds to” and analogous phrases refer to the ability of antibodies (or antigenically reactive fragments thereof) to bind specifically to analyte (or a fragment thereof without any substantial binding to other entities.

The bispecific antibodies of the present invention may contain two VH/VL pairs of different specificity on a single polypeptide chain, wherein the VH and VL domains in a respective scFv unit are separated by a polypeptide linker long enough to allow intramolecular association between these two domains, and wherein the thusly formed scFv units are contiguously tethered to one another through a polypeptide spacer kept short enough to prevent unwanted association between, for example, the VH domain of one scFv unit and the VL of the other scFv unit all while retaining the Fc portion present in full immunoglobulins. These bispecific antibodies are dual variable domains in tandem or Fab in Tandem. The bispecific antibodies may also be a heterodimer with common light chains.

The present invention utilizes methods for production of immunoglobulins, including, but not limited to full length antibodies and antibody fragments having a native sequence (i.e. that sequence produced in response to stimulation by an antigen), single chain antibodies which combine the antigen binding variable region of both the heavy and light chains in a single stably-folded polypeptide chain; univalent antibodies (which comprise a heavy chain/light chain dimer bound to the Fc region of a second heavy chain); “Fab fragments” which include the full “Y” region of the immunoglobulin molecule, i.e., the branches of the “Y”, either the light chain or heavy chain alone, or portions, thereof (i.e., aggregates of one heavy and one light chain, commonly known as Fab′); “hybrid immunoglobulins” which have specificity for two or more different antigens (e.g., quadromas or bispecific antibodies as described for example in U.S. Pat. No. 6,623,940); “composite immunoglobulins” wherein the heavy and light chains mimic those from different species or specificities; and “chimeric antibodies” wherein portions of each of the amino acid sequences of the heavy and light chain are derived from more than one species (i.e., the variable region is derived from one source such as a murine antibody, while the constant region is derived from another, such as a human antibody).

The bispecific antibody may be a Fab in Tandem (FIG. 12) (PCT International Publication No. WO/2015/103072).

### Vectors for Use in Practicing the Invention

The present invention contemplates the use of any plasmid or AAV viral vector serotype for introduction of constructs encoding immunoglobulin heavy and light chains and a self processing cleavage sequence into cells to achieve expression of immunoglobulin. A large number of mammalian host cell lines including NSO murine myeloma cells, PER.C6® human cells, and Chinese hamster ovary (CHO) cells (Feng 2010) and a large number of plasmid and AAV vectors are known in the art. In generating recombinant AAV viral vectors, non-essential genes are replaced with a gene encoding a protein or polypeptide of interest. The use of alternative AAV serotypes other than AAV2 (Davidson et al (2000), PNAS 97(7)3428-32; Passini et al (2003), J. Virol 77(12):7034-40) has demonstrated different cell tropisms and increased transduction capabilities. In one aspect, the present invention includes AAV vectors and methods that allow optimal AAV vector-mediated delivery and expression of an immunoglobulin or other therapeutic compound in vitro or in vivo. In one aspect, the present invention includes plasmid vectors and methods that allow optimal plasmid vector-mediated expression of an immunoglobulin or other therapeutic compound in vitro or in vivo.

A vector typically comprises an origin of replication and may or may not comprise a “marker” or “selectable marker” by means of which the vector can be identified and selected. While any selectable marker can be used, selectable markers for use in recombinant vectors are generally known in the art and the choice of the proper selectable marker will depend on the host cell. Examples of selectable marker genes which encode proteins that confer resistance to antibiotics or other toxins include, but are not limited to ampicillin, methotrexate, tetracycline, neomycin (Southern et al., J., J Mol Appl Genet. 1982; 1(4):327-41 (1982)), mycophenolic acid (Mulligan et al., Science 209:1422-7 (1980)), puromycin, zeomycin, hygromycin (Sugden et al., Mol Cell Biol. 5(2):410-3 (1985)) and G418. As will be understood by those of skill in the art, expression vectors typically include an origin of replication, a promoter operably linked to the coding sequence or sequences to be expressed, as well as ribosome binding sites, RNA splice sites, a polyadenylation site, and transcriptional terminator sequences, as appropriate to the coding sequence(s) being expressed.

Methotrexate (MTX) amplified CHO cells may be used to make antibodies useful in the present invention.

Reference to a vector or other DNA sequences as “recombinant” merely acknowledges the operable linkage of DNA sequences which are not operably linked in nature. Regulatory (expression and/or control) sequences are operatively linked to a nucleic acid coding sequence when the expression and/or control sequences regulate the transcription and, as appropriate, translation of the nucleotide sequence. Thus expression and/or control sequences can include promoters, enhancers, transcription terminators, a start codon (i.e., ATG) 5′ to the coding sequence, splicing signals for introns and stop codons.

Adeno-associated virus (AAV) is a helper-dependent human parvovirus which is able to infect cells latently by chromosomal integration. Because of its ability to integrate chromosomally and its nonpathogenic nature, AAV has significant potential as a human gene vector. For use in practicing the present invention rAAV virions may be produced using standard methodology, known to those of skill in the art and are constructed to include components operatively linked in the direction of transcription, control sequences including transcription initiation and termination sequences, the immunoglobulin coding sequence(s) of interest and a self processing cleavage sequence. The recombinant AAV vectors of the instant invention may specifically comprise: (1) a packaging site enabling the vector to be incorporated into replication-defective AAV virions; (2) the coding sequence for two or more polypeptides or proteins of interest, e.g., heavy and light chains of an immunoglobulin of interest; and (3) a sequence encoding a self-processing cleavage site alone or in combination with an additional proteolytic cleavage site. AAV vectors for use in practicing the invention are constructed such that they also include, as operatively linked components in the direction of transcription, control sequences including transcription initiation and termination sequences. These components are flanked on the 5′ and 3′ end by functional AAV ITR sequences. By “functional AAV ITR sequences” is meant that the ITR sequences function as intended for the rescue, replication and packaging of the AAV virion.

Adeno-associated virus (AAV) is a helper-dependent human parvovirus which is able to infect cells latently by chromosomal integration. Because of its ability to integrate chromosomally and its nonpathogenic nature, AAV has significant potential as a human gene therapy vector. For use in practicing the present invention rAAV virions may be produced using standard methodology, known to those of skill in the art and are constructed such that they include, as operatively linked components in the direction of transcription, control sequences including transcription initiation and termination sequences, the immunoglobulin coding sequence(s) of interest and a self processing cleavage sequence. The recombinant AAV vectors of the instant invention may specifically comprise: (1) a packaging site enabling the vector to be incorporated into replication-defective AAV virions; (2) the coding sequence for two or more polypeptides or proteins of interest, e.g., heavy and light chains of an immunoglobulin of interest; and (3) a sequence encoding a self-processing cleavage site alone or in combination with an additional proteolytic cleavage site. AAV vectors for use in practicing the invention are constructed such that they also include, as operatively linked components in the direction of transcription, control sequences including transcription initiation and termination sequences. These components are flanked on the 5′ and 3′ end by functional AAV ITR sequences. By “functional AAV ITR sequences” is meant that the ITR sequences function as intended for the rescue, replication and packaging of the AAV virion.

Recombinant AAV vectors are also characterized in that they are capable of directing the expression and production of recombinant immunoglobulins in target cells. Thus, the recombinant vectors comprise at least all of the sequences of AAV essential for encapsidation and the physical structures for infection of the recombinant AAV (rAAV) virions. Hence, AAV ITRs for use in the vectors of the invention need not have a wild-type nucleotide sequence (e.g., as described in Kotin, Hum. Gene Ther., 5:793-801, 1994), and may be altered by the insertion, deletion or substitution of nucleotides or the AAV ITRs may be derived from any of several AAV serotypes. Generally, an AAV vector is a vector derived from an adeno-associated virus serotype, including without limitation, AAV-1, AAV-2, AAV-3, AAV-4, AAV-5, AAV-6, AAV-7, AAV-8, etc. Preferred rAAV vectors have the wild type REP and CAP genes deleted in whole or part, but retain functional flanking ITR sequences. The Table below illustrates exemplary AAV serotypes for use in practicing the present invention.

TABLE
AAV Serotypes For Use In Gene Transfer.

| | | Genome | | Immunity |
|---|---|---|---|---|
| | | Size | Homology | in Human |
| Serotype | Origin | (bp) | to AAV2 | Population |
| AAV-1 | Human specimen | 4718 | NT: 80% | NAB: 20% |
| | | | AA: 83% | |
| AAV-2 | Human Genital | 4681 | NT: 100% | NAB: 27-53% |
| | Abortion Tissue | | AA: 100% | |
| | Amnion Fluid | | | |
| AAV-3 | Human | 4726 | NT: 82% | cross reactivity |
| | Adenovirus | | AA: 88% | with AAV2 NAB |
| | Specimen | | | |
| AAV-4 | African Green | 4774 | NT: 66% | Unknown |
| | Monkey | | AA: 60% | |
| AAV-5 | Human Genital | 4625 | NT: 65% | ELISA: 45% |
| | Lesion | | AA: 56% | NAB: 0% |
| AAV-6 | Laboratory | 4683 | NT: 80% | 20% |
| | Isolate | | AA: 83% | |
| AAV-7 | Isolated From | 4721 | NT: 78% | NAB: <1:20 (~5%) |
| | Heart DNA of | | AA: 82% | |
| | Rhesus Monkey | | | |
| AAV-8 | Isolated From | 4393 | NT: 79% | NAB: <1:20 (~5%) |
| | Heart DNA of | | AA: 83% | |
| | Rhesus Monkey | | | |

An AAV expression vector may be introduced into a producer cell, followed by introduction of an AAV helper construct, where the helper construct includes AAV coding regions capable of being expressed in the producer cell and which complement AAV helper functions absent in the AAV vector. The helper construct may be designed to down regulate the expression of the large Rep proteins (Rep78 and Rep68), typically by mutating the start codon following p5 from ATG to ACG, as described in U.S. Pat. No. 6,548,286, expressly incorporated by reference herein. This is followed by introduction of helper virus and/or additional vectors into the producer cell, wherein the helper virus and/or additional vectors provide accessory functions capable of supporting efficient rAAV virus production. The producer cells are then cultured to produce rAAV. These steps are carried out using standard methodology. Replication-defective AAV virions encapsulating the recombinant AAV vectors of the instant invention are made by standard techniques known in the art using AAV packaging cells and packaging technology. Examples of these methods may be found, for example, in U.S. Pat. Nos. 5,436,146; 5,753,500, 6,040,183, 6,093,570 and 6,548,286, expressly incorporated by reference herein in their entirety.

Further compositions and methods for packaging are described in Wang et al. (US 2002/0168342), also incorporated by reference herein in its entirety and include those techniques within the knowledge of those of skill in the art.

Approximately 40 serotypes of AAV are currently known, however, new serotypes and variants of existing serotypes are still being identified today and are considered within the scope of the present invention. See Gao et al (2002), PNAS 99(18):11854-6; Gao et al (2003), PNAS 100(10):6081-6; Bossis and Chiorini (2003), J. Virol. 77(12):6799-810). Different AAV serotypes are used to optimize transduction of particular target cells or to target specific cell types within a particular target tissue. The use of different AAV serotypes may facilitate targeting of diseased tissue. Particular AAV serotypes may more efficiently target and/or replicate in specific target tissue types or cells. A single self-complementary AAV vector can be used in practicing the invention in order to increase transduction efficiency and result in faster onset of transgene expression (McCarty et al., Gene Ther. 2001 August; 8(16):1248-54).

Host cells for producing rAAV virions include mammalian cells, insect cells, microorganisms and yeast. Host cells can also be packaging cells in which the AAV rep and cap genes are stably maintained in the host cell or producer cells in which the AAV vector genome is stably maintained and packaged. Exemplary packaging and producer cells are derived from 293, A549 or HeLa cells. AAV vectors are purified and formulated using standard techniques known in the art.

The vectors of the invention typically include heterologous control sequences, including, but not limited to, constitutive promoters, such as the cytomegalovirus (CMV) immediate early promoter, the RSV LTR, the MoMLV LTR, and the PGK promoter; tissue or cell type specific promoters including mTTR, TK, HBV, hAAT, regulatable or inducible promoters, enhancers, etc. Preferred promoters include the LSP promoter (Ill et al., Blood Coagul. Fibrinolysis 8S2:23-30 (1997)), the EF1-alpha promoter (Kim et al., Gene 91(2):217-23 (1990)) and Guo et al., Gene Ther. 3(9):802-10 (1996)). Most preferred promoters include the elongation factor 1-alpha (EF1a) promoter, a phosphoglycerate kinase-1 (PGK) promoter, a cytomegalovirus immediate early gene (CMV) promoter, chimeric liver-specific promoters (LSPs), a cytomegalovirus enhancer/chicken beta-actin (CAG) promoter, a tetracycline responsive promoter (TRE), a transthyretin promoter (TTR), a simian virus 40 (SV40) promoter and a CK6 promoter. The nucleotide sequences of these and numerous additional promoters are known in the art. The relevant sequences may be readily obtained from public databases and incorporated into AAV vectors for use in practicing the present invention.

### Delivery of Nucleic Acid Constructs Including Immunoglobulin Coding Sequences to Cells

The rAAV vector or plasmid vector constructs may comprise nucleotide sequences encoding antibodies or fragments thereof in the form of self-processing recombinant polypeptides and may be introduced into cells in vitro, ex vivo or in vivo for delivery of therapeutic genes to cells, e.g., somatic cells, or in the production of recombinant immunoglobulin by AAV vector-transduced or by plasmid vector-transduced cells.

The rAAV vector or plasmid vectors constructs may be introduced into cells in vitro or ex vivo using standard methodology known in the art. Such techniques include transfection using calcium phosphate, microinjection into cultured cells (Capecchi, Cell 22:479-488 (1980)), electroporation (Shigekawa et al., BioTechn., 6:742-751 (1988)), liposome-mediated gene transfer (Mannino et al., BioTechn., 6:682-690 (1988)), lipid-mediated transduction (Felgner et al., Proc. Natl. Acad. Sci. USA 84:7413-7417 (1987)), and nucleic acid delivery using high-velocity microprojectiles (Klein et al., Nature 327:70-73 (1987)).

The rAAV constructs or plasmid encoding constructs may be introduced into cells using standard infection or transfection techniques routinely employed by those of skill in the art.

For in vitro or ex vivo expression, any cell effective to express a functional immunoglobulin may be employed. Numerous examples of cells and cell lines used for protein expression are known in the art. For example, prokaryotic cells and insect cells may be used for expression. In addition, eukaryotic microorganisms, such as yeast may be used. The expression of recombinant proteins in prokaryotic, insect and yeast systems are generally known in the art and may be adapted for antibody expression using the compositions and methods of the present invention.

Examples of cells useful for immunoglobulin expression further include mammalian cells, such as fibroblast cells, cells from non-human mammals such as ovine, porcine, murine and bovine cells, insect cells and the like. Specific examples of mammalian cells include COS cells, VERO cells, HeLa cells, Chinese hamster ovary (CHO) cells, 293 cell, NSO cells, SP20 cells, 3T3 fibroblast cells, W138 cells, BHK cells, HEPG2 cells, DUX cells and MDCK cells.

Host cells are cultured in conventional nutrient media, modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium (MEM, Sigma), RPMI 1640 (Sigma), and Dulbecco's Modified Eagle's Medium (DMEM, Sigma) are typically suitable for culturing host cells. A given medium is generally supplemented as necessary with hormones and/or other growth factors (such as insulin, transferrin, or epidermal growth factor), DHFR, salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The appropriate culture conditions for a particular cell line, such as temperature, pH and the like, are generally known in the art, with suggested culture conditions for culture of numerous cell lines provided, for example, in the ATCC Catalogue available on line at <“http://www.atcc.org/Search catalogs/AllCollections.cfm”>. A rAAV vector or plasmid vector of the invention may be administered in vivo via any of a number of routes (e.g., intradermally, intravenously, intratumorally, into the brain, into the hand, into the skin, into the shoulder joint, into the uterine wall, intraportally, intraperitoneally, intramuscularly, into the bladder etc.), effective to deliver rAAV or plasmid vector in animal models or human subjects. Dependent upon the route of administration, the recombinant immunoglobulin will elicit an effect locally or systemically. The use of a tissue specific promoter 5′ to the immunoglobulin open reading frame(s) results in greater tissue specificity with respect to expression of a recombinant immunoglobulin expressed under control of a non-tissue specific promoter.

In vivo delivery of the recombinant AAV vectors may be targeted to a wide variety of organ types including, but not limited to brain, liver, blood vessels, muscle, heart, lung and skin. In vivo delivery of the recombinant AAV vectors of the invention may also be targeted to a wide variety of cell types based on the status of the cells. In the case of ex vivo gene transfer, the target cells are removed from the host and genetically modified in the laboratory using a recombinant AAV vector of the present invention and methods well known in the art.

The recombinant AAV vectors and or plasmid vectors of the invention can be administered using conventional modes of administration including but not limited to the modes described above and may be in a variety of formulations which include but are not limited to liquid solutions and suspensions, microvesicles, liposomes and injectable or infusible solutions. The preferred form depends upon the mode of administration and the therapeutic application.

The recombinant AAV vector constructs or plasmid vectors of the present invention may find utility in the in vitro production of recombinant antibodies for use in therapy. Methods for recombinant protein production are well known in the art and may be utilized for expression of recombinant antibodies using the self processing cleavage site-containing vector constructs described herein.

A recombinant immunoglobulin or fragment thereof, may be produced by introducing an AAV vector or plasmid vector such as described above into a cell to obtain an AAV-infected or plasmid-transfected cell, wherein the vector comprises in the 5′ to 3′ direction: a promoter operably linked to the coding sequence for an immunoglobulin heavy or light chain or fragment thereof, a self processing sequence such as a 2A or 2A-like sequence and the coding sequence for an immunoglobulin heavy or light chain or a fragment thereof, wherein the self processing cleavage sequence is inserted between the first and second immunoglobulin coding sequences. It will be appreciated that the coding sequence for either the immunoglobulin heavy chain or the coding sequence for the immunoglobulin light chain may be 5′ to the 2A sequence (i.e. first) in a given AAV construct or plasmid vector.

In one aspect, the invention provides a method for producing a recombinant immunoglobulin or fragment thereof, by introducing an AAV vector such as described above into a cell, wherein the AAV vector further comprises an additional proteolytic cleavage site between the first and second immunoglobulin coding sequences. A preferred additional proteolytic cleavage site is a furin cleavage site with the consensus sequence RXK(R)R (SEQ ID NO:10).

A cell for expressing a recombinant immunoglobulin or a fragment thereof, is provided wherein the cell comprises an AAV or DNA plasmid vector for the expression of two or more immunoglobulin chains or fragments thereof, a promoter operably linked to a first coding sequence for an immunoglobulin chain or fragment thereof, a self processing cleavage sequences, such as a 2A or 2A-like sequence, and a second coding sequence for an immunoglobulin chain or a fragment thereof, wherein the self processing cleavage sequence is inserted between the first and the second coding sequences. In a related aspect, the cell comprises an AAV vector or plasmid vector as described above wherein the expression vector further comprises an additional proteolytic cleavage site between the first and second immunoglobulin coding sequences. An additional proteolytic cleavage site is a furin cleavage site with the consensus sequence RXK(R)R (Arg Xaa Lys Arg Arg).

In the present invention, both DNA vectors and plasmid vectors that can be transfected into mammalian cells to make a stable recombinant antibody producing mammalian cell line may be used instead of AAV vector transfection.

“IL-33” refers to human interleukin-33, also known as IL-1F11, NF-HEV, and C9orf26. IL-33 is further described at online Mendelian Inheritance in Man (OMIM) entry 608678 and Gene ID No. 90865, and exemplary naturally occurring nucleic acid and protein sequences for human IL-33 are found at GenBank Accession Nos. NM_033439.2 (SEQ ID NO: 4) and AY905581 (SEQ ID NO: 6), respectively, all of which are available through the NCBI website. See also Schmitz et al. (2005) Immunity 23: 479-490. The contents of all database entries recited in the paragraph are hereby incorporated by reference in their entireties. A mature form of IL-33 has been proposed to be the active form of IL-33 (Schmitz et al. (2005) Immunity 23:479-490).

The minimal IL-33 receptor (IL-33R) complex consists of IL-1R4 (also known as IL-33Ra and ST2) and IL-1R3 (also known as IL-1RAcP) (Martin 2016).

Any known IL-33 antagonist may be utilized in the practice of this invention. The IL-33 antagonist preferably neutralizes biological function after binding. The IL-33 antagonist ANB020 is a functional anti-IL-33 therapeutic antibody currently in development. ANB020 inhibits IL-33 cytokine function by blocking interaction with the IL-33 cytokine's receptor at low picomolar potency. The IL-33 antagonist may be a decoy receptor, such as soluble ST2 (sST2 or soluble IL-1R4) (Kakkar 2008, Martin 2016). The decoy receptor preferably binds to and neutralizes biological function of the IL-33 receptor. The IL-33 antagonist may also be a soluble receptor (ST2/IL-1RAP) inhibitor. The IL-33 antagonist may also be a neutralizing antibody to IL-33, blocking antibodies to IL-IR4, or Fc-fusion proteins with the extracellular domain of IL-1R4 (Martin 2016). IL-33 antagonists may be administered at dosages between 0.001 mg/kg (mg IL-33 antagonist/kg of the subject's body weight) to 10 mg/kg, with preferable dosing in the range of 0.05 to 1 mg/kg.

Where the IL-33 antagonist comprises a bispecific (or bifunctional) antibody fragment or portion, the bispecific antibody or fragment thereof may comprise as one variable domain (e.g. antigen binding portion) an IL-33 antagonist and as the other variable domain (e.g. antigen binding portion) a second variable domain other than IL-33 antagonist. Optionally, the second variable domain may comprise a TNF antagonist, a GM-CSF antagonist, an IL-17 antagonist, an IL-21 antagonist or an IL-23 antagonist. A higher dose of IL-33 antagonist may be administered since the antibody or fragment thereof will be self-localising, minimizing systemic uptake and thus systemic side effects. Optionally, the second variable domain may comprise a DAMP antagonist (such as an antagonist for S100A8 and/or S100A9, e.g. as described in U.S. Pat. No. 7,553,488) or an AGE inhibitor (e.g. being variable domains of DAMP antagonist antibody or AGE inhibitor antibody). Methods for the production of bispecific (or bifunctional) antibodies, and bispecific (or bifunctional) antibody fragments are known in the art and are also discussed elsewhere in this application, which methods may be applied to the present purpose.

The IL-33 antagonist may be an antibody which specifically binds to a peptide comprising the amino acid sequence of SEQ ID NO. 23. The antibody may specifically bind to an epitope comprising the amino acid sequence of SEQ ID No. 24 or an epitope comprising the tetrapeptide sequence of SEQ ID NO. 25. These particular antibodies may be a polyclonal antibody, or alternatively, a monoclonal antibody. The binding of these antibodies may attenuate IL-33 activity, or inhibit IL-33 activity. The binding of these antibodies may prevent IL-33 activating the IL-33 receptor. Alternatively, the binding of these antibodies may promote proteolysis of IL-33, for example the antibody may be a catalytic antibody.

The IL-33 antagonist may be an antibody that alters the activity of IL-33 receptor bound IL-33 (IL-33-ST2), the antibody specifically binding to an epitope within the polypeptide sequence of SEQ ID NO. 26. The antibody may be a neutralising antibody. The binding of the antibody to IL-33 may prevent the ST2 (IL-4R) receptor from interacting with and/or associating with a co-receptor of the ST2-IL-33 receptor. For example, the binding of the antibody to IL-33 may prevent the ST2 receptor from interacting with and/or associating with IL-1 accessory protein. Alternatively, the binding of the antibody may prevent IL-33 from activating the ST2 receptor. These antibodies may be a polyclonal antibody or a monoclonal antibody. Although making of these antibodies is within the purview of a person of ordinary skill in the art, an example of making IL-33 antagonist is disclosed in U.S. Pat. No. 8,119,771, which is hereby incorporated by reference.

Antisense and siRNA molecules that reduce the expression of IL-33 may be designed based on the coding sequence for IL-33, as disclosed herein at SEQ ID NO: 4.

As an alternative to preparing monoclonal antibody-secreting hybridomas, a monoclonal anti-IL-33 antibody can be identified and isolated by screening a recombinant combinatorial immunoglobulin library (e.g., an antibody phage display library) with IL-33 to thereby isolate immunoglobulin library members that bind IL-33. Kits for generating and screening phage display libraries are commercially available for example, from Pharmacia and Stratagene. Additionally, examples of methods and reagents particularly amenable for use in generating and screening antibody display library can be found in, for example, Fuchs et al. (1991) Bio/Technology 9:1370-1372; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) PNAS 89:3576-3580; Hoogenboom et al. (1991) Nuc. Acid Res. 19:4133-4137; Barbas et al. (1991) PNAS 88:7978-7982; and McCafferty et al. Nature (1990) 348:552-554.

“IL-1R4” as used herein is synonymous with the T1/ST2 protein, and is also known as ST2, IL-1RL1, DER-4, Fit-1 and IL-1RL1. ST2 is further described at OMIM entry 601203 and Gene ID No. 9173. “Soluble ST2” (sST2) or “soluble IL-1R4” refers to a soluble extracellular variant of the membrane-bound form of ST2 (ST2L). Exemplary naturally occurring sequences for human sST2 are found at GenBank Accession Nos. NM_003856.2/NP_003847, and exemplary naturally occurring sequences for human ST2L are found at NM_016232.4/NP_057316, all of which are available through the NCBI website. The contents of all database entries recited in the paragraph are hereby incorporated by reference in their entireties. sST2 comprises residues 1-323 of ST2L and an additional C-terminal five amino acid residues (SKECF).

Soluble ST2 is believed to bind to IL-33 in solution, and thus to act as a natural antagonist of IL-33 by blocking binding to the IL-33 receptor of cell surfaces. Chackerian et al. (2007) J Immunol 179:2551-2555; Leung et al. (2004) J. Immunol. 173:145-150. Human sST2 comprises the sequence of amino acid residues 19-328 of SEQ ID NO: 5.

The IL-33 antagonist may be a soluble ST2 polypeptide, such as a polypeptide that includes amino acids 1-336 of SEQ ID NO: 22. The IL-33 antagonist may also include less than amino acids 1-336 of SEQ ID NO: 22 as long as it is a sufficient length to bind the naturally-occurring ligand IL-33. For example, the IL-33 antagonist may include at least about 8, 10, 12, 15, 20, 30, 50, 75, 100, 150, 200, 250, or 300, of amino acids 1-336 of SEQ ID NO: 22. Additionally, there may be up to 1, 2, 3, 4, 8, 10, 12, 15, 20, 30, 40 or 50, conservative amino acid substitutions.

While recombinant DNA techniques are the preferred method of producing useful soluble ST2 polypeptides having a sequence of more than 20 amino acids, shorter ST2 polypeptides having fewer than about 20 amino acids may also be produced by conventional chemical synthesis techniques. Synthetically produced polypeptides useful in the methods of this disclosure can be advantageously produced in extremely high yields and can be easily purified.

The IL-33 antagonist may be an isolated human or human-adapted antibody antagonist or fragment thereof that specifically binds Domain I (SEQ ID NO: 27) of human ST2L. An exemplary antibody binding Domain I of human ST2L (SEQ ID NO: 27) is an antibody STLM15 (C2244) comprising HCDR1, HCDR2 and HCDR3 sequences of SEQ ID NOs: 28, 29 and 30, respectively, and LCDR1, LCDR2 and LCDR3 sequences of SEQ ID NOs: 31, 32 and 33, respectively, or an antibody C2494 (STLM62) comprising HCDR1, HCDR2 and HCDR3 sequences of SEQ ID NOs: 34, 35 and 36, respectively, and LCDR1, LCDR2 and LCDR3 sequences of SEQ ID NOs: 37, 38 and 39, respectively (Table below).

| | HCDR1 | | HCDR2 | | |
|---|---|---|---|---|---|
| mAb | | SEQ ID | | SEQ ID | HCDR3 [symbol] |
| Name | Sequence | NO: | Sequence | NO: | Sequence |
| C2519A | DYMNM | | NINPYYGSTTYNQKFKG | | EGDTYLAWFAY |
| C2521A | TYWMN | | QIFPASGSTYYNEMFKD | | [symbol] NIYYINFQYYFAY [symbol] |
| C2244/STLM15 | SDYAWN | | FISYSGDTSFNPSLKS | | [symbol] GYSFDY |
| C2494/STLM62 | DDYMH | | RIDPAIGNTEYAPKFQD | | [symbol] FYAMDY |
| | LCDR1 | | LCDR2 | | |
| mAb | | SEQ ID | | SEQ ID | LCDR3 [symbol] |
| Name | Sequence | NO: | Sequence | NO: | Sequence |
| C2519A | RSSQSIVYSNGNTYLE | | KVSNRFS | | FQGSHVPPT |
| C2521A | RASQNIGTRMH | | YASESIS | | QQSNTWPFT |
| C2244/STLM15 | RASKSVSTSGSSYMF | | LASNLES | | QHSREIPYT |
| C2494/STLM62 | ITNTDIDDVIH | | EGNTLRP | | LQSDNMLT |

| mAb | |
|---|---|
| | VH sequence |
| C2519A | EFQLQQSGPELVKPGASVKISCKASGYSFTDYNMNWVKQSHGKSLEWI |
| | GNINPYYGSTTYNQKFKGKATLTVDKSSNTAYMHLNSLTSEDSAVYYC |
| | AREGDTYLAWFAYWGQGTLVTVSA |
| C2521A | QIQLQQSGPELVRPGTSVKISCKASGYTFLTYWMNWVKQRPGQGLEWI |
| | GQIFPASGSTYYNEMFKDKATLTVDTSSSTAYMQLSSLTSEDTAVYFC |
| | ARSENIYYINFQYYFAYWGQGTTLTVSS |
| C2244/STLM15 | EVQLQESGPGLVKPSQSLSLTCTVTGFSITSDYAWNWIRQFPGSKLEW |
| | MGFISYSGDTSFNPSLKSRISVTRDTSKNQFFLQLNSVTTEDTATYYC |
| | ASYDGYSFDYWGQGTTLTVSS |
| C2494/STLM62 | EVQLQQSVAELVRPGASVKLSCTASAFNIKDDYMHWVKQRPEQGLEWI |
| | GRIDPAIGNTEYAPKFQDKATMTADTSSNTAYLQLSSLTSEDTAVYYC |
| | ALGDFYAMDYWGQGTSVTVSS |
| | VL sequence |
| C2519A | DVLMTQTPLSLPVSLGDQASISCRSSQSIVYSNGNTYLEWYLQKPGQS |
| | PKLLIYKVSNRFSGVPDRFSGSGSGTDFTLKISRVEAEDLGVYYCFQG |
| | SHVPPTFGGGTKLEIK |
| C2521A | ILLTQSPAILSVSPGERVSFSCRASQNIGTRMHWYQQRTNGSPRLLIK |
| | YASESISGIPSRFSGSGSGTDFTLTISSVESEDIADYYCQQSNTWPFT |
| | FGSGTKLEIK |
| C2244/STLM15 | DIVLTQSPASLAISLGQRATISCRASKSVSTSGSSYMFWYQQKPGQPP |
| | KLLIYLASNLESGVPARFSGSGSGTDFTLNIHPVEEEDAAAYYCQHSR |
| | EIPYTFGGGTKLEIK |
| C2494/STLM62 | ETTVTQSPASLSVATGEKVTIRCITNTDIDDVIHWYQQKPGEPPKLLI |
| | SEGNTLRPGVPSRFSSSGYGTDFVFTIENTLSEDVADYYCLQSDNMLT |
| | FGAGTKLELK |

[symbol]

indicates data missing or illegible when filed

Additional exemplary antibodies binding Domain I of human ST2L are antibodies shown in the Table below and FIG. 24, for example antibodies STLM103, STLM107, STLM108, STLM123, STLM124, STLM208, STLM209, STLM210, STLM211, STLM212, and STLM213.

| | human ST2L affinity | | | cyno ST2L affinity | | | ST2L-ECD |
|---|---|---|---|---|---|---|---|
| | | | KD | | | KD | domain |
| | kₒₙ (M⁻¹s⁻¹) | k_{off} (s⁻¹) | (pM) | kₒₙ (M⁻¹s⁻¹) | k_{off} (s⁻¹) | (pM) | binding |
| STLM103 | 3.97E+06 | 1.63E−04 | 41 | 6.42E+06 | 2.02E−04 | 31 | D1 |
| STLM107 | 2.90E+07 | 3.41E−04 | 12 | 1.00E+08 | 6.50E−04 | 7 | D1 |
| STLM108 | 2.29E+06 | 2.22E−04 | 97 | 2.05E+07 | 5.98E−04 | 29 | D1 |
| STLM123 | 1.37E+07 | 2.08E−04 | 15 | 1.00E+08 | 5.19E−04 | 5 | D1 |
| STLM124 | 1.65E+07 | 7.56E−04 | 46 | 8.71E+07 | 2.57E−03 | 30 | D1 |
| STLM206 | 6.39E+06 | 1.60E−04 | 25 | 9.40E+07 | 5.83E−04 | 6 | D1 |
| STLM207 | 8.33E+06 | 3.95E−04 | 48 | 1.00E+08 | 2.07E−03 | 21 | D1 |
| STLM208 | 5.97E+06 | 6.76E−05 | 11 | 1.39E+07 | 7.02E−05 | 5 | D1 |
| STLM209 | 6.59E+06 | 1.70E−04 | 26 | 3.39E+07 | 3.11E−04 | 9 | D1 |
| STLM210 | 1.21E+07 | 2.27E−04 | 19 | 5.70E+07 | 5.28E−04 | 9 | D1 |
| STLM211 | 1.70E+07 | 4.83E−04 | 29 | 1.00E+08 | 1.39E−03 | 14 | D1 |
| STLM212 | 1.24E+07 | 3.98E−04 | 32 | 1.43E+07 | 3.46E−04 | 24 | D1 |
| STLM213 | 7.54E+06 | 1.08E−04 | 14 | 1.64E+07 | 1.24E−04 | 8 | D1 |
| STLM214 | 9.16E+06 | 2.99E−04 | 33 | 7.20E+06 | 2.64E−04 | 37 | D1 |
| STLM215 | 6.91E+06 | 1.72E−04 | 25 | 3.54E+07 | 3.69E−04 | 10 | D1 |
| STLM216 | 9.63E+06 | 1.58E−04 | 16 | 7.89E+07 | 2.64E−04 | 3 | D1 |
| STLM217 | 7.27E+06 | 1.26E−04 | 17 | 3.81E+07 | 1.38E−04 | 4 | D1 |
| STLM218 | 9.89E+06 | 2.24E−04 | 23 | 1.45E+07 | 2.65E−04 | 18 | D1 |
| STLM219 | 7.54E+06 | 2.01E−04 | 27 | 1.07E+07 | 2.30E−04 | 22 | D1 |
| STLM220 | 5.80E+06 | 9.53E−05 | 16 | 1.60E+07 | 1.40E−04 | 9 | D1 |
| STLM221 | 2.73E+06 | 9.61E−05 | 35 | 6.04E+06 | 1.30E−04 | 22 | D1 |
| STLM222 | 8.22E+06 | 3.01E−04 | 37 | 1.18E+07 | 3.45E−04 | 29 | D1 |
| STLM226 | 2.16E+07 | 1.93E−03 | 90 | 1.00E+08 | 3.01E−02 | 301 | D1 |
| STLM227 | 2.66E+07 | 1.70E−03 | 64 | 1.00E+08 | 2.94E−02 | 294 | D1 |
| STLM228 | 2.01E+07 | 1.04E−03 | 52 | 1.00E+08 | 1.55E−02 | 155 | D1 |
| STLM229 | 1.29E+07 | 4.45E−04 | 35 | 1.00E+08 | 8.50E−03 | 85 | D1 |
| STLM230 | 1.11E+07 | 4.26E−04 | 38 | 5.06E+07 | 7.30E−03 | 144 | D1 |
| STLM231 | 1.97E+07 | 9.13E−04 | 46 | 8.27E+07 | 1.43E−02 | 172 | D1 |
| STLM232 | 1.78E+07 | 4.49E−04 | 25 | 1.00E+08 | 7.97E−03 | 80 | D1 |

Exemplary human antibody antagonists are shown in FIG. 25 and FIG. 24. Exemplary human-adapted antagonists are shown in the Table below.

| | | VH chains | | |
|---|---|---|---|---|
| | | | >VH2494- | >VH2494- |
| | | Parent* | IGHV1-24*01 | IGHV1-f*01 |
| VL chains | pRD# | pDR4211 | pDR9870 | pDR9871 |
| Parent* | pDR4212 | STLM126 | STLM186 | STLM196 |
| >VL2494-IGKV1-39*01 O12b | pDR9865 | STLM127 | STLM187 | STLM197 |
| >VL2494-IGKV3-15*01 L2 | pDR9873 | STLM129 | STLM189 | STLM199 |
| >VL2494-IGKV1-9*01 L8 | pDR9874 | STLM130 | STLM190 | STLM200 |
| >VL2494-IGKV1-5*01 L12 | pDR9875 | STLM131 | STLM191 | STLM201 |
| >VL2494-IGKV1-12*01 L5 | pDR9876 | STLM132 | STLM192 | STLM202 |
| >VL2494-IGKV1-39*01 O12 | pDR9877 | STLM133 | STLM193 | STLM203 |
| >VL2494-IGKV1-27*01 A20 | pDR9878 | STLM134 | STLM194 | STLM204 |
| >VL2494-IGKV1-33*01 O18 | pDR9879 | STLM135 | STLM195 | STLM205 |

*Parent = C2494 VH and VL

Any known TNF antagonist may be utilized in the practice of the invention, a broad variety of which are known and disclosed in the art. The TNF antagonist preferably neutralizes biological function after binding. The TNF antagonist is preferably a human TNF antagonist. Optionally, the TNF antagonist may be an antibody, such as a monoclonal antibody or fragment thereof; a chimeric monoclonal antibody (such as a human-murine chimeric monoclonal antibody); a fully human monoclonal antibody; a recombinant human monoclonal antibody; a humanized antibody fragment; a soluble TNF antagonist, including small molecule TNF blocking agents such as thalidomide or analogues thereof or PDE-IV inhibitors; a TNF receptor or a TNF receptor fusion protein, e.g. a soluble TNFR1 (p55) or TNFR2 (p75) TNF receptor or TNF receptor fusion protein. Optionally, the TNF antagonist is a functional fragment or fusion protein comprising a functional fragment of a monoclonal antibody, e.g. of the 15 types mentioned above, such as a Fab, F(ab′)2, Fv and preferably Fab. Preferably a fragment is pegylated or encapsulated (e.g. for stability and/or sustained release). The TNF antagonist may also be a camelid antibody. As used herein, TNF antagonists include but are not limited to TNF receptor inhibitors.

Preferably, the TNF antagonist is selected from those which at administration (e.g. local administration, such as injection into: a clinical nodule or cord of Dupuytren's disease, a localized deposit endometriosis, the inflammatory nodule in adhesive capsulitis, hypertrophic scar, or keloid scar) cause administration-site irritation manifested as palpable local swelling, redness and pruritis in fewer than 40% of patients, preferably fewer than 20% and more preferably fewer than 10%.

The TNF antagonist may be selected, for example, from one or a combination of Infliximab, Adalimumab, Certolizumab pegol, Golimumab or Etanercept, or functional fragment thereof.

A humanized binding protein or antigen binding fragment thereof comprising an antigen binding domain capable of binding TNF, the antigen binding domain comprising a heavy chain variable region (VH) and a light chain variable region (VL), wherein the VH region comprises an amino acid sequence selected from the group consisting of SEQ ID NOs: 7, 8, 9, 10, and 11, and the VL region comprises an amino acid sequence selected from the group consisting of SEQ ID NOs: 12, 13, and 14 may be used as part of the bispecific antibody of the present invention. Additionally, the antibody, or antigen binding portion thereof, capable of binding TNF in the bispecific antibody may comprises an amino acid sequence selected from the group consisting of SEQ ID NOs: 15-21.

Anti hTNF Antibodies

The Table below is a list of amino acid sequences of VH and VL regions (CDR sequences bolded) of anti-hTNF-antibodies of the invention.

TABLE
List of Amino Acid Sequences of Murine
Anti-hTNF-α Antibody VH And VL Regions

| SEQ | | | |
|---|---|---|---|
| ID | | | Sequence |
| NO. | Protein region | | 123456789012345678901234567890 |
| 15 | VH | | QVQLKESGPGLVAPSQSLSITCTVSGFSLT |
| | MAK195 | | DYGVNWVRQPPGKGLEWLGMIWGDGSTDYD |
| | | | STLKSRLSISKDNSKSQIFLKMNSLQTDDT |
| | | | ARYYCAREWHHGPVAYWGQGTLVTVSA |
| | VH | Residues 31-35 | DYGVN |
| | MAK195CDR-H1 | of SEQ ID NO: 15 | |
| | VH | Residues 50-65 | MIWGDGSTDYDSTLKS |
| | MAK195CDR-H2 | of SEQ ID NO: 15 | |
| | VH | Residues 98-106 | EWHHGPVAY |
| | MAK195CDR-H3 | of SEQ ID NO: 15 | |
| 16 | VL | | DIVMTQSHKFMSTTVGDRVSITCKASQAVS |
| | MAK195 | | SAVAWYQQKPGQSPKLLIYWASTRHTGVPD |
| | | | RFTGSGSVTDFTLTIHNLQAEDLALYYCQQ |
| | | | HYSTPFTFGSGTKLEIKR |
| | VL | Residues 24-34 | KASQAVSSAVA |
| | MAK195CDR-L1 | of SEQ ID NO: 16 | |
| | VL | Residues 50-56 | WASTRHT |
| | MAK195CDR-L2 | of SEQ ID NO: 16 | |
| | VL | Residues 89-97 | QQHYSTPFT |
| | MAK195CDR-L3 | of SEQ ID NO: 16 | |

### Anti-hTNF Chimeric Antibodies

A chimeric antibody is a molecule in which portions of the antibody are derived from different animal species, such as antibodies having a variable region derived from a murine monoclonal antibody and a human immunoglobulin constant region. Methods for producing chimeric antibodies are known in the art. See, e.g., Morrison (1985) Science 229:1202; Oi et al. (1986) BioTechniques 4:214-221; Gillies et al. (1989) J. Immunol. Methods 125:191-202; U.S. Pat. Nos. 5,807,715; 4,816,567; and 4,816,397. In addition, techniques developed for the production of “chimeric antibodies” (Morrison et al. (1984) Proc. Natl. Acad. Sci. USA 81:6851-6855; Neuberger et al. (1984), Nature 312:604-608; Takeda et al. (1985) Nature 314:452-454; by splicing genes from a mouse antibody molecule of appropriate antigen specificity together with genes from a human antibody molecule of appropriate biological activity can be used.

The chimeric antibodies disclosed herein may be produced by replacing the heavy chain constant region of the murine monoclonal anti human TNF-antibodies described above with a human IgG1 constant region.

### Anti-TNF CDR-Grafted Antibodies

CDR-grafted antibodies may comprise heavy and light chain variable region sequences from a human antibody wherein one or more of the CDR regions of VH and/or VL are replaced with CDR sequences of the murine antibodies of the invention. A framework sequence from any human antibody may serve as the template for CDR grafting. However, straight chain replacement onto such a framework often leads to some loss of binding affinity to the antigen. The more homologous a human antibody is to the original murine antibody, the less likely the possibility that combining the murine CDRs with the human framework will introduce distortions in the CDRs that could reduce affinity. Therefore, the human variable framework that is chosen to replace the murine variable framework apart from the CDRs may have at least a 65%, 70%, 75%, or 80% sequence identity with the murine antibody variable region framework. Methods for producing chimeric antibodies are known in the art (also see EP Patent No. EP 0 239 400; PCT Publication WO 91/09967; U.S. Pat. Nos. 5,225,539; 5,530,101; and 5,585,089), veneering or resurfacing (EP 0 592 106; EP 0 519 596; Padlan (1991) Mol. Immunol. 28(4/5):489-498; Studnicka et al. (1994) Protein Eng. 7(6):805-814; Roguska et al. (1994) Proc. Natl. Acad. Sci. USA 91:969-973), and chain shuffling (U.S. Pat. No. 5,565,352).

Select CDR grafted antibodies with VH and/or VL chains are described in the Table below.

CDR Grafted Antibodies

| | Sequence |
|---|---|
| Protein region | 123456789012345678901234567890 |
| hMAK195VH.1z | QVQLQESGPGLVKPSETLSLTCTVSGGSIS |
| | DYGVNWIRQPPGKGLEWIGMIWGDGSTDYD |
| | STLKSRVTISVDTSKNQFSLKLSSVTAADT |
| | AVYYCAREWHHGPVAYWGQGTLVTVSS |
| hMAK195VH.2z | EVQLVESGGGLIQPGGSLRLSCAASGFTVS |
| | DYGVNWVRQAPGKGLEWVSMIWGDGSTDYD |
| | STLKSRFTISRDNSKNTLYLQMNSLRAEDT |
| | AVYYCAREWHHGPVAYWGQGTLVTVSS |
| hMAK195Vk.1 | DIQMTQSPSSLSASVGDRVTITCKASQAVS |
| | SAVAWYQQKPGKAPKLLIYWASTRHTGVPS |
| | RFSGSGSGTDFTLTISSLQPEDFATYYCQQ |
| | HYSTPFTFGQGTKLEIK |
| hMAK195Vk.2z | EIVNTQSPATLSVSPGERATLSCKASQAVS |
| | SAVAWYQQKPGQAPRLLIYWASTRHTGIPA |
| | RFSGSGSGTEPTLTISSLQSEDFAVYYCQQ |
| | HYSTPFTPGQGTKLEIK |

Humanized antibodies are antibody molecules from non-human species antibody that binds the desired antigen having one or more complementarity determining regions (CDRs) from the non-human species and framework regions from a human immunoglobulin molecule. Known human Ig sequences are disclosed, e.g., www.ncbi.nlm.nih.gov/entrez-/query.fcgi; www.atcc.org/phage/hdb.html; www.sciquest.com/; www.abcam.com/; www.antibodyresource.com/onlinecomp.html; www.public.iastate.edu/.about.pedro/research_tools.html; www.mgen.uni-heidelberg.de/SD/IT/IT.html; www.whfreeman.com/immunology/CH-05/kuby05.htm; www.library.thinkquest.org/12429/Immune/Antibody.html; www.hhmi.org/grants/lectures/1996/vlab/; www.path.cam.ac.ukLabout.mrc7/m-ikeimages.html; www.antibodyresource.com/; mcb.harvard.edu/BioLinks/Immunology.html.www.immunologylink.com/; pathbox.wustl.edu/.about.hcenter/index.-html; www.biotech.ufl.edu/.about.hcl/; www.pebio.com/pa/340913/340913.html-; www.nal.usda.gov/awic/pubs/antibody/; www.m.ehime-u.acjp/.about.yasuhito-/Elisa.html; www.biodesign.com/table.asp; www.icnet.uk/axp/facs/davies/lin-ks.html; www.biotech.ufl.edu/.about.fccl/protocol.html; www.isac-net.org/sites_geo.html; aximtl.imt.uni-marburg.de/.about.rek/AEP-Start.html; baserv.uci.kun.nl/.about.jraats/linksl.html; www.recab.uni-hd.de/immuno.bme.nwu.edu/; www.mrc-cpe.cam.ac.uk/imt-doc/public/INTRO.html; www.ibt.unam.mx/virN_mice.html; imgt.cnusc.fr:8104/; www.biochem.ucl.ac.uk/.about.martin/abs/index.html; antibody.bath.ac.uk/; abgen.cvm.tamu.edu/lab/wwwabgen.html; www.unizh.ch/.about.honegger/AHOseminar/Slide01.html; www.cryst.bbk.ac.ukLabout.ubcg07s/; www.nimr.mrc.ac.uk/CC/ccaewg/ccaewg.htm; www.path.cam.ac.ukl.about.mrc7/humanisation/TAHHP.html; www.ibt.unam.mx/vir/structure/stat_aim.html; www.biosci.missouri.edu/smithgp/index.html; www.cryst.bioc.cam.ac.uk/.about.fmolina/Web-pages/Pept/spottech.html; wwwjerini.de/frroducts.htm; www.patents.ibm.com/ibm.html.Kabat et al., Sequences of Proteins of Immunological Interest, U.S. Dept. Health (1983). Such imported sequences can be used to reduce immunogenicity or reduce, enhance or modify binding, affinity, on-rate, off-rate, avidity, specificity, half-life, or any other suitable characteristic, as known in the art.

Framework residues in the human framework regions may be substituted with the corresponding residue from the CDR donor antibody to alter, improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., U.S. Pat. No. 5,585,089; Riechmann et al. (1988) Nature 332:323-327.) Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding. Antibodies can be humanized using a variety of techniques known in the art, such as but not limited to those described in Jones et al. (1986) Nature 321:522-525; Verhoeyen et al. (1988) Science 239:1534-1536; Sims et al. (1993) J. Immunol. 151: 2296-2308; Chothia and Lesk (1987) J. Mol. Biol. 196:901-917; Carter et al. (1992) Proc. Natl. Acad. Sci. USA 89:4285-4289; Presta et al. (1993) J. Immunol. 151:2623-2632; Padlan (1991) Mol. Immunol. 28(4/5):489-498; Studnicka et al. (1994) Protein Engineering 7(6):805-814; Roguska. et al. (1994) Proc. Natl. Acad. Sci. USA 91:969-973; PCT Publication Nos. WO 91/09967, WO 99/06834 (International Application No. PCT/US98/16280), WO 97/20032 (Appln. No. PCT/US96/18978), WO 92/11272 (Appln. No. PCT/US91/09630), WO 92/03461 (Appln. No. PCT/US91/05939), WO 94/18219 (Appln. No. PCT/US94/01234), WO 92/01047 (Appln. No. PCT/GB91/01134), WO 93/06213 (Appln. No. PCT/GB92/01755), WO 90/14443, WO 90/14424, and WO 90/14430; European Publication Nos. EP 0 592 106, EP 0 519 596, and EP 0 239 400, U.S. Pat. Nos. 5,565,332; 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

As used herein, “aptamer” means a nucleic acid molecule with a binding affinity for a predetermined target molecule. The aptamer may be an RNA, a DNA, a modified nucleic acid or a mixture thereof. In general, aptamers are short stretches of nucleotides.

The terms “IL-33/TNF aptamer construct” and “TNF/IL-33 aptamer construct” are used to refer to a construct comprising a IL-33 aptamer and a TNF aptamer. The order of the words “IL-33” and “TNF” in “IL-33/TNF aptamer construct” and “TNF/IL-33 aptamer construct” is not indicative of how the aptamers are linked, e.g., the order does not indicate which aptamer is located at the 5′ position of an aptamer construct and which aptamer is located at the 3′ position in the aptamer construct. In some embodiments, a IL-33/TNF aptamer construct is capable of binding TNF and IL-33 simultaneously. In some embodiments, a IL-33/TNF aptamer construct is capable of binding each of IL-33 and TNF separately. In a IL-33/TNF aptamer construct, the IL-33 aptamer and the TNF aptamer may be linked covalently or non-covalently, e.g., through a binding pair such as streptavidin and biotin. A IL-33/TNF aptamer construct may comprise a linker between the TNF aptamer and the IL-33 aptamer.

Any IL-33 aptamer which functions as an antagonist may be used in the practice of this invention. The IL-33 aptamer antagonist preferably neutralizes biological function after binding. The IL-33 aptamer antagonist may: specifically bind to, and inhibit activation of, an IL-33 receptor; be in soluble form that specifically binds to IL-33 and inhibits IL-33 from binding to the IL-33 receptor; or inhibit synthesis of IL-33. Aptamer antagonists and methods of making and using them are described in the following: US Patent Application Publications: 2017/0227550; 2017/0166895; 2017/0107292; 2017/0051337; 2015/0168423; 2014/0315986; 2014/0303018; 2014/0249043; 2014/0201965; 2014/0155411; 2014/0135302; 2014/0081011; 2013/0012693; 2012/0322862, 2012/0264117; 2012/0231467; 2012/0115752; 2011/0245479; 2011/0136099; 2011/0082286; 2010/0317120; 2010/0221752; 2009/0098549; 2009/0042206; 2009/0004667; 2008/0160535; 2006/0057573; 2004/0132067; 2004/0106145; 2003/0087301; 2002/0106652; and U.S. Pat. Nos. 5,989,823; 6,171,795; 6,242,246; 6,261,783; 6,291,184; 6,376,190; 6,329,145; 6,458,539; 6,458,453; 6,482,594; 6,503,715; 6,531,286; 6,544,776; 6,569,620; 6,706,482; 6,730,482; 6,670,132; 6,673,553; 7,629,151; 6,706,482; 6,716,580; 7,709,192; 7,855,054; 7,947,447; 7,964,356; 8,404,830; 8,409,795; 8,598,140; 8,703,416; 8,925,175; 8,945,830; 8,975,026; 8,975,388; 8,925,175; 9,081,010; 9,103,837; 9,163,056; 9,206,429; 9,314,797; 9,316,647; 9,365,855; 9,382,533; 9,404,919; 9,410,156; 9,423,403; 9,612,248; 9,651,556; 9,695,424; and 9,701,967 which are hereby incorporated by reference into this application.

For example, a method to identify aptamers that selectively bind the IL-33 receptor or fragments thereof may comprise contacting the target protein IL-33, with a library of candidate aptamers, and selectively identifying/isolating/enriching for aptamers that specifically bind IL-33.

Any TNF aptamer which functions as an antagonist may be used in the practice of this invention. The TNF aptamer antagonist preferably neutralizes biological function after binding. The TNF aptamer antagonist may: specifically bind to, and inhibit activation of, a TNF receptor; be in soluble form that specifically binds to TNF and inhibits TNF from binding to the TNF receptor; or inhibit synthesis of TNF. Aptamer antagonists and methods of making and using them are described in the publications identified above.

For example, a method to identify aptamers that selectively bind the TNF receptor or fragments thereof may comprise contacting the target protein TNF, with a library of candidate aptamers, and selectively identifying/isolating/enriching for aptamers that specifically bind TNF.

Any known GM-CSF (Granulocyte-macrophage colony-stimulating factor) antagonist may be utilized in the implementation of this invention. The GM-CSF antagonist preferably neutralizes biological function after binding. The GM-CSF antagonist may be: an antibody, or antigen binding fragment of an antibody, that specifically binds to, and inhibits activation of, an GM-CSF receptor; a soluble form of an GM-CSF receptor that specifically binds to GM-CSF and inhibits GM-CSF from binding to the GM-CSF receptor; an antisense nucleic acid that specifically inhibits synthesis of GM-CSF; a siRNA that specifically inhibits synthesis of GM-CSF; a small molecule that specifically inhibits the activity of GM-CSF; or a bispecific antibody comprising at least one antigen binding domain of which binds to and inhibits activation of, an GM-CSF receptor. The GM-CSF antagonist may be an antibody and the antibody is a chimeric antibody, a humanized antibody, a human antibody or an antigen binding fragment of chimeric humanized and human antibody. Examples of GM-CSF antagonists include, but are not limited to, E21R and E21K. Other examples of GM-CSF antagonists are described in U.S. 8,398,972, the contents of which are hereby incorporated by reference.

Any known IL-17 (Interleukin 17) antagonist may be utilized in the implementation of this invention. The IL-17 antagonist preferably neutralizes biological function after binding. The IL-17 antagonist may be: an antibody, or antigen binding fragment of an antibody, that specifically binds to, and inhibits activation of, an IL-17 receptor; a soluble form of an IL-17 receptor that specifically binds to IL-17 and inhibits IL-17 from binding to the IL-17 receptor; an antisense nucleic acid that specifically inhibits synthesis of IL-17; a siRNA that specifically inhibits synthesis of IL-17; a small molecule that specifically inhibits the activity of IL-17; or a bispecific antibody comprising at least one antigen binding domain of which binds to and inhibits activation of, an IL-17 receptor. The IL-17 antagonist may be an antibody wherein the antibody is a chimeric antibody, a humanized antibody, a human antibody, and an antigen binding fragment of a chimeric humanized and human antibody. Examples of IL-17 antagonists include, but are not limited to, secukinumab, brodalumaband, and ixekizumab. Other examples of IL-17 antagonists are described in PCT International Publication Nos. WO2012045848A1 and WO2012059598A2, the contents of which are hereby incorporated by reference.

Any known IL-21 (Interleukin 21) antagonist may be utilized in the implementation of this invention. The IL-21 antagonist preferably neutralizes biological function after binding. The IL-21 antagonist may be: an antibody, or antigen binding fragment of an antibody, that specifically binds to, and inhibits activation of, an IL-21 receptor; a soluble form of an IL-21 receptor that specifically binds to IL-21 and inhibits IL-21 from binding to the IL-21 receptor; an antisense nucleic acid that specifically inhibits synthesis of IL-21; a siRNA that specifically inhibits synthesis of IL-21; a small molecule that specifically inhibits the activity of IL-21; or a bispecific antibody comprising at least one antigen binding domain of which binds to and inhibits activation of, an IL-21 receptor. The IL-21 antagonist may be an antibody selected from the group consisting of chimeric antibodies, humanized antibodies, human antibodies, and antigen binding fragments of chimeric humanized and human antibodies. Examples of IL-21 antagonists are described in U.S. Pat. No. 7,923,539, and PCT International Publication Nos. WO 2007/114861 and WO 2003040313 A2, the contents of which are hereby incorporated by reference.

Any known IL-23 (Interleukin 23) antagonist may be utilized in the implementation of this invention. The IL-23 antagonist preferably neutralizes biological function after binding. The IL-23 antagonist may be: an antibody, or antigen binding fragment of an antibody, that specifically binds to, and inhibits activation of, an IL-23 receptor; a soluble form of an IL-23 receptor that specifically binds to IL-23 and inhibits IL-23 from binding to the IL-23 receptor; an antisense nucleic acid that specifically inhibits synthesis of IL-23; a siRNA that specifically inhibits synthesis of IL-23; a small molecule that specifically inhibits the activity of IL-23; or a bispecific antibody comprising at least one antigen binding domain of which binds to and inhibits activation of, an IL-23 receptor. The IL-23 antagonist may be an antibody selected from the group consisting of chimeric antibodies, humanized antibodies, human antibodies, and antigen binding fragments of chimeric humanized and human antibodies. Examples of IL-23 antagonists include, but are not limited to, ustekinumab and briakinumab. Other examples of IL-23 antagonists are described in PCT International Publication No. WO 2007147019 the contents of which are hereby incorporated by reference.

An RNA interference (RNAi) antagonist is an RNA molecule that modulates or inhibits gene expression. Vector-derived RNAi (AAV-RNAi) is where a vector is used to express RNA transcripts (e.g., short-hairpin RNAs (shRNAs) or micro RNAs (miRNAs) that are ultimately processed to produce siRNAs in the target cells. The present invention includes using an AAV-RNAi to inhibit IL-33, TNF, and both IL-33 and TNF together.

By early disease, early disease stage, or early disease state it is meant that indications of disease are present, e.g. histological markers or more particularly clinical nodules in tissue, but in the absence of, for example, significant contracture. By early Dupuytren's disease, early disease stage Dupuytren's disease or early disease state Dupuytren's disease, it is meant that indications of Dupuytren's disease are present, for example histological markers or more particularly clinical nodules in palmar and/or digital tissue, and a flexion deformity of less than or equal to 30 degrees at any joint in the digit.

By established disease stage or established disease state, it is meant that clinical nodules are present, palpable cord is present and contracture is evident. By established disease stage Dupuytren's disease, it is meant that clinical nodules are present on the palm and digits of the hand and a flexion deformity of greater than 30 degrees at any joint in the digit.

Varying histological stages of Dupuytren's disease have been categorised in the literature, most succinctly by Rombouts (1989) and later authors, into three distinct stages: 1) a proliferative stage with high cellularity and the presence of mitotic figures; 2) a fibrocellular stage characterised by high cellularity but no mitotic figures and the presence of reticulin network; and 3) a fibrous stage with few cells separated by broad bundles of collagen fibres. Stage 1) disease is believed to correlate with early disease stage as discussed above (i.e. presence of nodules but no contracture) and Dupuytren's stages 2) and 3) is believed to correlate with our Established Disease Stage (characterized by digital contracture). During early and early established disease stages, active myofibroblasts are collected in the established nodules and cords, especially in relation to the MCP and PIP joints and these drive the progression of flexion contractures of the digit.

In certain claims, the invention claims the amount of the TNF antagonist as a multiple of the clinical dose administered for Rheumatoid Arthritis. For example, if a claim states the TNF antagonist is administered in an amount between about 0.05 and about 5.0 times the clinical dose of the TNF antagonist typically administered to a patient with rheumatoid arthritis, and the clinical dose administered for Rheumatoid Arthritis for that particulate TNF antagonist is 100 mg, then the dose of the TNF antagonist for the claimed method is between 5 mg and 500 mg.

The antagonists of the present invention may be injected directly into the affected tissue. The antagonists of the present invention may be injected to a site of maximal cellularity or maximal inflammation.

The antagonist may be administered by intra articular injection, peri articular injection, systemic injection (IV), or subcutaneous injection (SC) to a patient with periarticular fibrosis. The antagonist may be administered by intralesional (anterior shoulder capsule/coracohumeral ligament), intra articular injection, peri articular injection, systemic injection (IV) or subcutaneous injection (SC) to a patient with frozen shoulder. The antagonist may be administered by intralesional injection, systemic injection (IV) or subcutaneous injection (SC) to a patient with cutaneous scarring (keloid & hypertrophic). The antagonist may be administered by intra-peritoneal injections, by systemic injection (IV), by subcutaneous injection (SC), or in a formulation that coats the bowel serosal and peritoneal surface to a patient with abdominal adhesions. The antagonist may be administered by intralesional injection, by intra-peritoneal injections, by systemic injection (IV), by subcutaneous injection (SC) or in a formulation that coats the pelvic and or abdominal organ surface to a patient with endometriosis. The antagonist may be administered to the tissue surrounding an implant to a patient with fibrosis around implants.

### Production of Bispecific Antibodies

Bispecific antibodies with defined dual specificity suitable for therapeutic use must be generated through biochemical or genetic means. Bispecific binding proteins can be generated using cell fusion, chemical conjugation, or recombinant DNA techniques. Bispecific antibodies have been produced using quadroma technology (see Milstein, C. and A. C. Cuello (1983) Nature 305(5934):537-40) based on the somatic fusion of two different hybridoma cell lines expressing murine monoclonal antibodies (mAbs) with the desired specificities of the bispecific antibody. Because of the random pairing of two different immunoglobulin (Ig) heavy and light chains within the resulting hybrid-hybridoma (or quadroma) cell line, up to ten different Ig species are generated, of which only one is the functional bispecific antibody. The presence of mis-paired by-products, and significantly reduced production yields, means sophisticated purification procedures are required.

Bispecific antibodies can also be produced by chemical conjugation of two different monoclonal antibodies (mAbs) (see Staerz, U. D., et al. (1985) Nature 314(6012): 628-31). This approach does not yield homogeneous preparation. Other approaches have used chemical conjugation of two different mAbs or smaller antibody fragments (see Brennan, M., et al. (1985) Science 229(4708): 81-3).

Bispecific antibodies may also be produced by knob-into-hole or similar approaches, which introduce mutations in the Fc region (see Holliger, P. et al. (1993) Proc. Natl. Acad. Sci USA 90(14): 6444-6448), resulting in multiple different immunoglobulin species of which only one is the functional bispecific antibody.

Another method used to produce bispecific antibodies is the coupling of two parental antibodies with a hetero-bifunctional crosslinker, but the resulting bispecific antibodies suffer from significant molecular heterogeneity because reaction of the crosslinker with the parental antibodies is not site-directed. To obtain more homogeneous preparations of bispecific antibodies two different Fab fragments have been chemically crosslinked at their hinge cysteine residues in a site-directed manner (see Glennie, M. J., et al. (1987) J. Immunol. 139(7): 2367-75). But this method results in Fab′2 fragments, not full IgG molecule.

A wide variety of other recombinant bispecific antibody formats have been developed (see Kriangkum, J., et al. (2001) Biomol. Eng. 18(2): 31-40). Amongst them tandem single-chain Fv molecules and diabodies, and various derivatives thereof, are the most widely used. Routinely, construction of these molecules starts from two single-chain Fv (scFv) fragments that recognize different antigens (see Economides, A. N., et al. (2003) Nat. Med. 9(1): 47-52). Tandem scFv molecules (taFv) represent a straightforward format simply connecting the two scFv molecules with an additional peptide linker. The two scFv fragments present in these tandem scFv molecules form separate folding entities. Various linkers can be used to connect the two scFv fragments and linkers with a length of up to 63 residues (see Nakanishi, K., et al. (2001) Ann. Rev. Immunol. 19: 423-74). Although the parental scFv fragments can normally be expressed in soluble form in bacteria, it is, however, often observed that tandem scFv molecules form insoluble aggregates in bacteria. Hence, refolding protocols or the use of mammalian expression systems are routinely applied to produce soluble tandem scFv molecules. In a recent study, in vivo expression by transgenic rabbits and cattle of a tandem scFv directed against CD28 and a melanoma-associated proteoglycan was reported (see Gracie, J. A., et al. (1999) J. Clin. Invest. 104(10): 1393-401). In this construct, the two scFv molecules were connected by a CH1 linker and serum concentrations of up to 100 mg/L of the bispecific antibody were found. Various strategies including variations of the domain order or using middle linkers with varying length or flexibility were employed to allow soluble expression in bacteria. A few studies have now reported expression of soluble tandem scFv molecules in bacteria (see Leung, B. P., et al. (2000) J. Immunol. 164(12): 6495-502; Ito, A., et al. (2003) J. Immunol. 170(9): 4802-9; Karni, A., et al. (2002) J. Neuroimmunol. 125(1-2): 134-40) using either a very short Ala3 linker or long glycine/serine-rich linkers. In another recent study, phage display of a tandem scFv repertoire containing randomized middle linkers with a length of 3 or 6 residues was employed to enrich for those molecules that are produced in soluble and active form in bacteria. This approach resulted in the isolation of a tandem scFv molecule with a 6 amino acid residue linker (see Arndt, M. and J. Krauss (2003) Methods Mol. Biol. 207: 305-21). It is unclear whether this linker sequence represents a general solution to the soluble expression of tandem scFv molecules. Nevertheless, this study demonstrated that phage display of tandem scFv molecules in combination with directed mutagenesis is a powerful tool to enrich for these molecules, which can be expressed in bacteria in an active form.

The bispecific antibodies of the present invention be made by any process disclosed in this application or otherwise known in the art.

Diabodies are the recombinant bispecific antibodies (BsAbs), constructed from heterogeneous single-chain antibodies. Bispecific diabodies (Db) utilize the diabody format for expression. Diabodies are produced from scFv fragments by reducing the length of the linker connecting the VH and VL domain to approximately 5 residues (see Peipp, M. and T. Valerius (2002) Biochem. Soc. Trans. 30(4): 507-11). This reduction of linker size facilitates dimerization of two polypeptide chains by crossover pairing of the VH and VL domains. Bispecific diabodies are produced by expressing, two polypeptide chains with, either the structure VHA-VLB and VHB-VLA (VH-VL configuration), or VLA-VHB and VLB-VHA (VL-VH configuration) within the same cell. A large variety of different bispecific diabodies have been produced in the past and most of them are expressed in soluble form in bacteria. However, a recent comparative study demonstrates that the orientation of the variable domains can influence expression and formation of active binding sites (see Mack, M. et al. (1995) Proc. Natl. Acad. Sci. USA 92(15): 7021-5). Nevertheless, soluble expression in bacteria represents an important advantage over tandem scFv molecules. However, since two different polypeptide chains are expressed within a single cell inactive homodimers can be produced together with active heterodimers. This necessitates the implementation of additional purification steps in order to obtain homogenous preparations of bispecific diabodies. One approach to force the generation of bispecific diabodies is the production of knob-into-hole diabodies (see Holliger, P., T. Prospero, and G. Winter (1993) Proc. Natl. Acad. Sci. USA 90(14): 6444-8.18). This approach was demonstrated for a bispecific diabody directed against HER2 and CD3. A large knob was introduced in the VH domain by exchanging Val37 with Phe and Leu45 with Trp and a complementary hole was produced in the VL domain by mutating Phe98 to Met and Tyr87 to Ala, either in the anti-HER2 or the anti-CD3 variable domains. By using this approach the production of bispecific diabodies could be increased from 72% by the parental diabody to over 90% by the knob-into-hole diabody. Importantly, production yields only slightly decrease as a result of these mutations. However, a reduction in antigen-binding activity was observed for several constructs. Thus, this rather elaborate approach requires the analysis of various constructs in order to identify those mutations that produce heterodimeric molecule with unaltered binding activity. In addition, such approach requires mutational modification of the immunoglobulin sequence at the constant region, thus creating non-native and non-natural form of the antibody sequence, which may result in increased immunogenicity, poor in vivo stability, as well as undesirable pharmacokinetics.

Single-chain diabodies (scDb) represent an alternative strategy for improving the formation of bispecific diabody-like molecules (see Holliger, P. and G. Winter (1997) Cancer Immunol. Immunother. 45(3-4): 128-30; Wu, A. M., et al. (1996) Immunotechnology 2(1): p. 21-36). Bispecific single-chain diabodies are produced by connecting the two diabody-forming polypeptide chains with an additional middle linker with a length of approximately 15 amino acid residues. Consequently, all molecules with a molecular weight corresponding to monomeric single-chain diabodies (50-60 kDa) are bispecific. Several studies have demonstrated that bispecific single chain diabodies are expressed in bacteria in soluble and active form with the majority of purified molecules present as monomers (see Holliger, P. and G. Winter (1997) Cancer Immunol. Immunother. 45(3-4): 128-30; Wu, A. M., et al. (1996) Immunotechnol. 2(1): 21-36; Pluckthun, A. and P. Pack (1997) Immunotechnol. 3(2): 83-105; Ridgway, J. B., et al. (1996) Protein Engin. 9(7): 617-21). Thus, single-chain diabodies combine the advantages of tandem scFvs (all monomers are bispecific) and diabodies (soluble expression in bacteria).

More recently diabodies have been fused to Fc to generate more Ig-like molecules, named di-diabodies (see Lu, D., et al. (2004) J. Biol. Chem. 279(4): 2856-65). In addition, multivalent antibody construct comprising two Fab repeats in the heavy chain of an IgG and capable of binding four antigen molecules has been described (see WO 0177342A1, and Miller, K., et al. (2003) J. Immunol. 170(9): 4854-61).

Bispecific antibodies his can be achieved through the use of domains attached to peptides which self-associate to form homo-multimers (Pack & Pluckthun, 1992, Biochemistry 31, 1579-1584). For example, the association of two separately expressed scFv antibody fragments by C-terminally fused amphipathic helices in vivo provides homodimers of antibody fragments in E. coli (PCT/EP93/00082; Pack et al., 1993, Bio/Technology 11, 1271-1277) or homo-tetramers; (Pack et al., 1995, J. Mol. Biol., 246, 28-34).

The bispecific antibody may also comprise individually encoded peptides or “segments” which, in a single continuous chain, would comprise a compact tertiary structure with a highly hydrophobic core. The component peptides are chosen so as to be asymmetric in their assumed structure, so as not to self-associate to form homo-multimers, but rather to associate in a complementary fashion, adopting a stable complex which resembles the parent tertiary structure. On the genetic level, these segments are encoded by interchangeable cassettes with suitable restriction sites. These standardized cassettes are fused C- or N-terminally to different recombinant proteins via a linker or hinge in a suitable expression vector system. The structure of this bispecific antibody or multifunctional polypeptide is described schematically in FIG. 14. Polypeptide segments which do not have the ability to assemble as homodimers are derived by cutting a parental polypeptide which has a compact tertiary structure and a highly hydrophobic core. These polypeptide segments can then fused to one or more different functional domains at the genetic level. These distinct polypeptide segments which are now fused to one or more functional domains can be, for example, coexpressed resulting in the formation of a native like parental structure attached to functional domains. This parental structure is formed by the dimerization of the polypeptide segments which were derived from the original parental polypeptide. The resulting multifunctional complex, as pictured in FIG. 14, would appear as a compact tertiary structure attached to the one or more functional domains.

Once structural sub-domains are identified, the protein is dissected in such a way these sub-domains remain intact. The selection process can be expanded to proteins for which no structure is available but which satisfy the criteria of stability and good expression. For these proteins, folding sub-domains can be determined by hydrogen exchange pulse-labelling of backbone amides during the folding reaction, followed by NMR detection in the native state (Roder et al., 1988, Nature 355, 700-704; Udgaonkar & Baldwin, 1988, Science 255, 594-597). Alternatively, folding sub-domains can be identified by mild proteolysis, denaturation, purification of fragments and reconstitution in vitro (Tasayco & Carey, 1992, Science 255, 594-597; Wu et al., 1993, Biochemistry 32, 10271-10276). Finally, additional clues for the choice of cleavage sites can be obtained from the exon structure in the case of eukaryotic proteins, since the exons frequently (though not always) correspond to structural sub-domains of a protein. This has, for example, been discussed for the case of myoglobin (Go 1981, Nature 291, 90).

As part of this invention, DNA sequences, vectors, preferably bicistronic vectors, vector cassettes, may be made and characterized in that they comprise a DNA sequence encoding an amino acid sequence and optionally at least one further (poly)peptide comprised in the multifunctional polypeptide of the invention, and additionally at least one, preferably singular cloning sites for inserting the DNA encoding at least one further functional domain or that they comprise DNA sequences encoding the amino acid sequences, and optionally the further (poly)peptide(s) comprised in the multifunctional polypeptide of the invention and suitable restriction sites for the cloning of DNA sequences encoding the functional domains, such that upon expression of the DNA sequences after the insertion of the DNA sequences encoding the functional domains into said restriction sites, in a suitable host the multifunctional polypeptide of the invention may be formed. Said vector cassette is characterized in that it comprises the inserted DNA sequence(s) encoding said functional domain(s) and host cells transformed with at least one vector or vector cassette of the invention which can be used for the preparation of said bispecific or multi-functional polypeptides. The host cell may be a mammalian, preferably human, yeast, insect, plant or bacterial, preferably E. coli cell.

The bispecific antibodies of the present invention may be prepared by a method which comprises culturing at least two host cells of the invention in a suitable medium, said host cells each producing only one of said first and said second amino acid sequences attached to at least one further functional domain, recovering the amino acid sequences, mixing thereof under mildly denaturing conditions and allowing in vitro folding of the multifunctional polypeptide of the invention from said amino acid sequences.

The method may be characterized in that the further amino acid sequences attached to at least one further functional domain are/is produced by at least one further host cell not producing said first or second amino acid sequence.

Additionally, the method may be characterized in that at least one further amino acid sequence attached to at least one further functional domain is produced by the host cell of the invention producing said first or second amino acid sequence.

The present invention may include pharmaceutical and diagnostic compositions comprising the multi-functional polypeptides described above, said pharmaceutical compositions optionally comprising a pharmaceutically acceptable carrier.

Antibodies may be generated using Prokaryotic Host Cells as described in U.S. Patent Publication No. 2015/0315290.

There are several classes of bispecific antibodies, such as Asymmetric IgG-Like (FIG. 15), Symmetric IgG-Like (FIG. 16), IgG Fusions (FIG. 17), Fc Fusions (FIG. 18), Fab Fusions (FIG. 19), ScFv- and Diabody-based (FIG. 20), IgG/Non-IgG Fusions (FIG. 21), heterodimeric IgG bispecifics (including Quadroma Triomab, Knobs-into-holes In Vitro assembly, Common LC, CrossMab^{CH1-CL}, (SEED) body, and LUZ-Y) (FIG. 22), bispecific Abs from fragments (FIG. 23).

To make the antibodies of the present invention, fully human IgGs may be expressed in highly engineered yeast strain of S. Cerevisiae. The IgGs on the surface of yeast can then be interrogated for antigen binding, IgG pairing, stability, and expression.

Steps that may be used to create a bispecific antibody include a computation assessment, usage of a curated data set, an In silico design, a synthetic DNA, an IgG library and screening diversity. In the development of bispecific antibodies, a Human IgG Library may be used to synthetically mimic natural V-D-J and V-J recombination processes. The Human IgG Library has unique diversity sets, and consists of >20 VH and VL common human germlines covering multiple canonical structures. An assessment by Flow Cytometer yields Affinity of Purified IgGs which is a tool that can be used to select for preferred activity.

The process of preparing the bispecific antibody may comprise (1) discovering an initial panel of IgGs and used for initial functional testing, (2) performing a biology assessment so that IgGs can help further understand target biology and define goals for useful therapeutic, and (3) optimization of the bispecific antibody.

Bispecific antibodies may also be classified into the following five distinct structural groups: (i) bispecific IgG (BsIgG) (ii) IgG appended with an additional antigen-binding moiety (iii) BsAb fragments (iv) bispecific fusion proteins and (v) BsAb conjugates as described in Speiss 2015 (FIG. 13).

When either the second or the first portion of a bispecific antibody of the invention comprises two antibody variable domains, these two antibody variable domains may be a VH- and VL-domain which are associated with one another. However, it is also contemplated that the two antibody variable domains comprised in either the second or the first portion may be two VH domains or two VL regions which are associated with one another. In the event that the two antibody variable domains of the first or second portion are covalently associated with one another, the two antibody variable domains may be designed as an scFv fragment, meaning that the two domains are separated from one another by a peptide linker long enough to allow intermolecular association between these two domains. The design of linkers suitable for this purpose is described in the prior art, for example in the granted patents EP 623 679 B1, U.S. Pat. No. 5,258,498, EP 573 551 B1 and U.S. Pat. No. 5,525,491. In other words, a bispecific antibody may be a construct with a total of three antibody variable domains. One antibody variable domain specifically binds alone, i.e., without being paired with another antibody variable domain (a) either to a human immune effector cell by specifically binding to an effector antigen on the human immune effector cell or to a target cell, while the remaining two antibody variable domains together specifically bind (b) either to the target antigen on the target cell or to a human immune effector cell by specifically binding to an effector antigen on the human immune effector cell, respectively.

In this case, the presence of three antibody variable domains in the bispecific antibody entails unique advantages. Often, an scFv exhibiting the desired binding specificity for a target antigen is already known and optimized, and omitting one of its two antibody variable domains would abolish or at least attenuate its binding characteristics. Such an scFv may make up part of this bispecific antibody. Specifically, such a three-domain antibody may advantageously comprise an entire scFv as either its effector antigen- or target antigen-conferring portion.

Effectively, then, this allows a bispecific antibody to be formed starting from a desired scFv by simple incorporation of only one additional antibody variable domain into the same polypeptide chain as the scFv, wherein the one additional antibody variable domain incorporated has an antigen binding specificity different than that of the scFv.

In this context, it has been found that such incorporation of a third antibody variable domain to form a three-domain bispecific single chain antibody leads to the same, or substantially the same, production characteristics as described for the two-domain bispecific antibodies of this invention. For example, problems such as low yield, restriction to complicated expression systems, heterogeneous products, etc., recounted above for bispecific antibodies with four antibody variable domains pose little to no problem when expressing these three-domain bispecific antibodies.

The first and second portions of the bispecific antibody may be separated from one another by a synthetic polypeptide spacer moiety, which covalently (i.e., peptidically) links either the C-terminus of the first portion with the N-terminus of the second portion, or the C-terminus of the second portion with the N-terminus of the first portion. As such, the portions of these bispecific antibodies may be arranged, as either N-(first portion)-(second portion)-C or N-(second portion)-(first portion)-C.

A large group of recombinant bispecific antibodies are IgGlike molecules. In most of these formats, binding sites of a second specificity are fused to the N- or C-terminus of the heavy or light chain, e.g., in the form of an scFv fragment or a variable single domain, resulting in bispecific, tetravalent molecules. Bispecific molecules generated through fusion of an scFv fragment to a mAb offer great flexibility. ScFv molecules have been fused to the N-terminus but also the C-terminus of the heavy or light chain of a mAb, generally without compromising productivity or antigen-binding activity. This group of IgG-like bispecific molecules also includes DVD-Igs, where a second VH and VL domain is fused to the heavy and light chain, respectively, of a mAb, two-in-one antibodies, where a second specificity is introduced into the natural binding site of an IgG molecule, and mAb2 molecules, where a second specificity is built into the CH3 domain of the Fc region. A characteristic feature of all these molecules is a symmetry caused by dimeric assembly of two identical heavy chains, an intrinsic property of these chains. A different approach is the generation of asymmetric IgG molecules. This can be achieved with the knobs-into holes strategy. Here, amino acids at the contact site between the CH3 domains are substituted by larger or smaller residues forcing a heterodimeric assembly of heavy chains. The random association with the light chains is addressed by generating bispecific molecules with common light chains, or, by domain swapping between one heavy and light chain resulting in CrossMabs. Heavy chain heterodimerization is achieved by engineering a charged CH3 interface to introduce an electrostatic steering effect or using the strand-exchange engineered domain technology (SEEDbody) with CH3 sequences composed of alternating segments from human IgA and IgG. In contrast to the bispecific IgG-like molecules, these bispecific antibodies are bivalent with a size basically identical to that of IgG. Fc heterodimerization was recently applied to generate a trivalent, bispecific molecule fusing a VH and a VL domain to the C-termini of the engineered heavy chains (HA-TF Fc variant.) Bispecific antibodies with a molecular mass in the range of 50-100 kDa can be generated by combining the variable domains of two antibodies. For example, two scFv have been connected by a more or less flexible peptide linker in a tandem orientation (tandem scFv, taFv, tascFv), which can be extended further by additional scFv, e.g., generating bispecific or trispecific triple bodies (sctb). Diabodies are heterodimeric molecules composed of the variable domains of two antibodies arranged either in the order VHA-VLB and VHB-VLA (VH-VL orientation) or in the order VLA-VHB and VLB-VHA (VL-VH orientation). The linker connecting the two domains within one chain is approximately 5 residues leading, after co-expression of the two chains within one cell, to a head-to-tail assembly and hence formation of a compact molecule with two functional binding sites. The diabody (Db) format was further stabilized by introducing interchain disulfide bonds (dsDb, DART molecules) or by generating a single-chain derivative (scDb). ScDbs can be converted into tetravalent molecules by reducing the middle linker, resulting in homodimerzation of two chains. Small bispecific molecules have also been produced by fusing a scFv to the heavy or light chain of a Fab fragment. Furthermore, tandem scFv, diabodies and scDb have been fused to the Fc or a CH3 domain to generate tetravalent derivatives. Also, scFv can be combined with Fc or CH3 domains to generate tetravalent molecules, e.g., fusing scFvs to the N- and C-terminus of an Fc fragment, or using the knobs-into-holes approach to generate bivalent scFv-Fc or scFv-CH3 molecules. A different approach for the generation of bispecific antibodies of the present invention is the dock-and-lock method (DNL). Here, antibody fragments are fused to a homodimerizing docking domain (DDD) from human cAMP-dependent protein kinase A (PKA) and the anchoring domain (AD) from A-kinase anchor protein (AKAP) leading to the formation of bispecific, trivalent molecules. Many of the established bispecific antibody formats can also be combined with additional proteins and components, e.g., drugs, toxins, enzymes and cytokines, enabling dual targeting and delivery of a fusion partner. In addition, fusion to plasma proteins such as serum albumin or albumin-binding moieties can be applied to extend the plasma half-life of bispecific antibodies.

### Structure of Bispecific Antibodies

In one example the bispecific antibody may be a binding protein comprising a polypeptide chain, wherein the polypeptide chain comprises VD1-(X1)n-VD2-C-(X2)n, wherein VD1 is a first variable domain, VD2 is a second variable domain, C is a constant domain, X1 represents an amino acid or polypeptide, X2 represents an Fc region and n is 0 or 1. The VD1 and VD2 in the binding protein may be heavy chain variable domains selected from the group consisting of a murine heavy chain variable domain, a human heavy chain variable domain, a CDR grafted heavy chain variable domain, and a humanized heavy chain variable domain. VD1 and VD2 may be capable of binding different antigens. C may be a heavy chain constant domain. For example, X1 is a linker with the proviso that X1 is not CH1. For example, X1 is a linker listed herein. In an embodiment, X2 is an Fc region. In another embodiment, X2 is a variant Fc region.

Antibodies of the invention specifically binding TNF or 11-33 can be engineered into bispecific antibodies which are also encompassed within the scope of the invention. The VL and/or the VH regions of the antibodies of the invention can be engineered using published methods into single chain bispecific antibodies as structures such as T and Ab® designs (Int. Pat. Publ. No. WO1999/57150; U.S. Pat. Publ. No. US2011/0206672) or into bispecific scFVs as structures such as those disclosed in U.S. Pat. No. 5,869,620; Int. Pat. Publ. No. WO1995/15388A, int. Pat. Publ. No. WO1997/14719 or Int. Pat. Publ. No WO2011/036460.

The VL and/or the VH regions of the IL-33 and TNF antibodies of the invention can be engineered into bispecific full length antibodies, Such bispecific antibodies are typically made by modulating the CH3 interactions between the two antibody heavy chains to form bispecific antibodies using technologies such as those described in U.S. Pat. No. 7,695,936; Int. Pat. Publ. No. WO04/111233; U.S. Pat. Publ. No. US2010/0015133; U.S. Pat. Publ. No. US2007/0287170; Int. Pat. Publ. No. WO2008/119353; U.S. Pat. Publ. No. US2009/0182127; U.S. Pat. Publ. No. US2010/0286374; U.S. Pat. Publ. No. US2011/0123532; Int. Pat. Publ. No. WO2011/131746; Int. Pat. Publ. No. WO2011/143545; or U.S. Pat. Publ. No. US2012/0149876. Additional bispecific structures into which the VL and/or the VH regions of the antibodies of the invention can be incorporated are for example Dual Variable Domain Immunoglobulins (Int. Pat. Publ. No. WO2009/134776), or structures that include various dimerization domains to connect the two antibody arms with different specificity, such as leucine zipper or collagen dimerization domains (Int. Pat. Publ. No. WO2012/022811, U.S. Pat. Nos. 5,932,448; 6,833,441).

### Dual Targeting Strategies

Dual targeting strategies using bispecific antibodies can be divided into two types: (i) those that directly act on target structures, e.g., cell surface receptors or soluble factors and (ii) those that use dual targeting for delivery (retargeting) of a therapeutically active moiety, e.g., effector molecules and effector cells. Direct actions include binding and neutralization of two ligands or two receptors, neutralization of a receptor and a ligand, activation of two receptors, activation of one receptor and neutralization of another receptor or a soluble factor, but also neutralization by binding to different epitopes of one receptor or ligand (FIG. 26, A-H). Indirect actions include ADCC and CDC mediated by an Fc region, retargeting of immune effector cells through a further binding site, targeting of an effector molecule, e.g., a toxin, a cytokine or a prodrug-converting enzyme and targeting of drug-loaded nanoparticles (FIG. 26, I-O). Direct and indirect actions can be combined within one molecule to further improve efficacy. Applications of dual targeting strategies are likewise manifold, with the main indications being cancer therapy and the treatment of inflammatory and infectious diseases. Here, the same mechanisms used for combination therapy of antibodies can be targeted with bispecific antibodies. Multiple diseases mediators and signaling pathways thus can be addressed and simultaneously inhibited by the dual targeting antibody. This includes targets that act independently on different pathways, but also targets that are capable of cross-talking.

The bispecific antibodies of the present invention may also be prepared by joining a single chain antibody (ScFv) after the C terminus (CH3-ScFv) or after the hinge (Hinge-ScFv) with an antibody of a different specificity as described in Coloma 1996. The fusion is effected at the DNA level and the engineered gene expressed by transfection.

Bispecific antibodies can be also produced by genetic engineering and more than 45 different formats have been established in the past two decades (FIG. 27).

### Method of Producing a Dual-Variable Domain Immunoglobulin

The bispecific antibodies of the present invention may also be a dual-variable domain immunoglobulin (DVD-Ig™) as described in Jakob 2013 which combines the target binding domains of two monoclonal antibodies via flexible naturally occurring linkers, which yields a tetravalent IgG-like molecule.

Production of the Hinge-ScFv and CH3-ScFv expression vectors. To produce the anti-TNF/anti-IL-33 bispecific antibody, an anti-IL-33 single chain antibody (ScFv) gene is first produced by PCR. The ScFv is then ligated through a short flexible linker after the hinge or CH3 of a human IgG3 gene and joined to a TNF heavy chain variable region in an expression vector. The expression vectors for the anti-TNF-Hinge-ScFv and the anti-TNF-CH3-ScFv are co-transfected with an anti-TNF V_{L} expression vector into P3X63Ag8.563 myeloma cells.

The invention additionally provides a method of making a DVD-Ig binding protein by preselecting the parent antibodies of Il-33 and TNF. A method of making a Dual Variable Domain Immunoglobulin that binds two antigens comprises the steps of a) obtaining a first parent antibody, or antigen binding portion thereof, that binds a first antigen; b) obtaining a second parent antibody or antigen binding portion thereof, that binds a second antigen; c) constructing first and third polypeptide chains, each of which comprises VD1-(X1)n-VD2-C-(X2)n, wherein, VD1 is a first heavy chain variable domain obtained from said first parent antibody, or antigen binding portion thereof; VD2 is a second heavy chain variable domain obtained from said second parent antibody or antigen binding portion thereof, which can be the same as or different from the first parent antibody; C is a heavy chain constant domain; (X1)n is a linker with the proviso that it is not CH1, wherein said (X1)n is either present or absent; and (X2)n is an Fc region, wherein said (X2)n is either present or absent; d) constructing second and fourth polypeptide chains each of which comprises VD1-(X1)n-VD2-C-(X2)n, wherein, VD1 is a first light chain variable domain obtained from said first parent antibody, or antigen binding portion thereof; VD2 is a second light chain variable domain obtained from said second parent antibody, or antigen binding thereof, which can be the same as or different from the first parent antibody; C is a light chain constant domain; (X1)n is a linker with the proviso that it is not CH1, wherein said (X1)n is either present or absent; and (X2)n does not comprise an Fc region, wherein said (X2)n is either present or absent; and e) expressing said first, second, third and fourth polypeptide chains; such that a DVD-Ig binds said first antigen and said second antigen is generated.

The invention provides another method of generating a DVD-Ig that binds two antigens with desired properties, which comprises the steps of a) obtaining a first parent antibody (for example, an IL-33 antibody), or antigen binding portion thereof, that binds a first antigen and possessing at least one desired property exhibited by the DVD-Ig; b) obtaining a second parent antibody (for example, TNF antibody), or antigen binding portion thereof, can bind to a second antigen and possesses at least one desired property exhibited by the Dual Variable Domain Immunoglobulin; c) constructing first and third polypeptide chains comprising VD1-(X1)n-VD2-C-(X2)n, wherein; VD1 is a first heavy chain variable domain obtained from said first parent antibody, or antigen binding portion thereof; VD2 is a second heavy chain variable domain obtained from said second parent antibody, or antigen binding portion thereof; C is a heavy chain constant domain; (X1)n is a linker with the proviso that it is not CH1, wherein said (X1)n is either present or absent; and (X2)n is an Fc region, wherein said (X2)n is either present or absent; d) constructing second and fourth polypeptide chains comprising VD1-(X1)n-VD2-C-(X2)n, wherein; VD1 is a first light chain variable domain obtained from said first parent antibody, or antigen binding portion thereof; VD2 is a second light chain variable domain obtained from said second parent antibody, or antigen binding portion thereof), which can be the same as or different from the first parent antibody; C is a light chain constant domain; (X1)n is a linker with the proviso that it is not CH1, wherein said (X1)n is either present or absent; and (X2)n does not comprise an Fc region, wherein said (X2)n is either present or absent; e) expressing said first, second, third and fourth polypeptide chains; such that a Dual Variable Domain Immunoglobulin capable of binding said first and said second antigen with desired properties is generated.

### Generation of a DVD Binding Protein, an Illustrative Example

The bispecific antibodies of the present invention may be Dual Variable Domain binding proteins that bind one or more targets. These binding proteins may comprise a polypeptide chain, wherein said polypeptide chain comprises VD1-(X1)n-VD2-C-(X2)n, wherein VD1 is a first variable domain, VD2 is a second variable domain, C is a constant domain, X1 represents an amino acid or polypeptide, X2 represents an Fc region and n is 0 or 1. The following processes may be used to prepare such a binding protein.

### A. Generation of Parent Monoclonal Antibodies:

The variable domains of the DVD binding protein can be obtained from parent antibodies, including polyclonal and mAbs that bind antigens of interest. These antibodies may be naturally occurring or may be generated by recombinant technology.

MAbs can be prepared using a wide variety of techniques known in the art including the use of hybridoma, recombinant, and phage display technologies, or a combination thereof. For example, mAbs can be produced using hybridoma techniques including those known in the art and taught, for example, in Harlow et al. (1988) Antibodies: A Laboratory Manual, (Cold Spring Harbor Laboratory Press, 2nd ed.); Hammerling, et al. (1981) in: Monoclonal Antibodies and T-Cell Hybridomas 563-681 (Elsevier, N.Y.). The term “monoclonal antibody” is not limited to antibodies produced through hybridoma technology. The term “monoclonal antibody” refers to an antibody that is derived from a single clone, including any eukaryotic, prokaryotic, or phage clone, and not the method by which it is produced. Hybridomas are selected, cloned and further screened for desirable characteristics, including robust hybridoma growth, high antibody production and desirable antibody characteristics. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art. The hybridomas may be mouse hybridomas and/or may be produced in a non-human, non-mouse species such as rats, sheep, pigs, goats, cattle or horses. The hybridomas may also be human hybridomas, in which a human non-secretory myeloma is fused with a human cell expressing an antibody that binds a specific antigen.

Recombinant mAbs are also generated from single, isolated lymphocytes using a procedure referred to in the art as the selected lymphocyte antibody method (SLAM), as described in U.S. Pat. No. 5,627,052; PCT Publication No. WO 92/02551; and Babcock, J. S. et al. (1996) Proc. Natl. Acad. Sci. USA 93:7843-7848. In this method, single cells secreting antibodies of interest, e.g., lymphocytes derived from an immunized animal, are identified, and, heavy- and light-chain variable region cDNAs are rescued from the cells by reverse transcriptase-PCR. These variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example by panning the transfected cells to isolate cells expressing antibodies to the antigen of interest. The amplified immunoglobulin sequences further can be manipulated in vitro, such as by in vitro affinity maturation methods such as those described in PCT Publication Nos. WO 97/29131 and WO 00/56772.

Monoclonal antibodies are also produced by immunizing a non-human animal comprising some, or all, of the human immunoglobulin locus with an antigen of interest. In an embodiment, the non-human animal is a XENOMOUSE transgenic mouse, an engineered mouse strain that comprises large fragments of the human immunoglobulin loci and is deficient in mouse antibody production. See, e.g., Green et al. (1994) Nature Genet. 7: 13-21 and U.S. Pat. Nos. 5,916,771; 5,939,598; 5,985,615; 5,998,209; 6,075,181; 6,091,001; 6,114,598; and 6,130,364. See also PCT Publication Nos. WO 91/10741; WO 94/02602; WO 96/34096; WO 96/33735; WO 98/16654; WO 98/24893; WO 98/50433; WO 99/45031; WO 99/53049; WO 00/09560; and WO 00/037504. The XENOMOUSE transgenic mouse produces an adult-like human repertoire of fully human antibodies, and generates antigen-specific human monoclonal antibodies. The XENOMOUSE transgenic mouse contains approximately 80% of the human antibody repertoire through introduction of megabase sized, germline configuration YAC fragments of the human heavy chain loci and x light chain loci. See Mendez et al. (1997) Nature Genet. 15: 146-156; Green and Jakobovits (1998) J. Exp. Med. 188: 483-495.

In vitro methods also can be used to make the parent antibodies, wherein an antibody library is screened to identify an antibody having the desired binding specificity. Methods for such screening of recombinant antibody libraries are well known in the art and include methods described in, for example, Ladner et al., U.S. Pat. No. 5,223,409; PCT Publication Nos. WO 92/18619; WO 91/17271; WO 92/20791; WO 92/15679; WO 93/01288; WO 92/01047; WO 92/09690 and WO 97/29131; Fuchs et al. (1991) Bio/Technology 9: 1370-1372; Hay et al. (1992) Hum. Antibod. Hybridomas 3: 81-85; Huse et al. (1989) Science 246: 1275-1281; McCafferty et al. (1990) Nature 348: 552-554; Griffiths et al. (1993) EMBO J. 12: 725-734; Hawkins et al. (1992) J. Mol. Biol. 226: 889-896; Clackson et al. (1991) Nature 352: 624-628; Gram et al. (1992) Proc. Natl. Acad. Sci. USA 89: 3576-3580; Garrad et al. (1991) Bio/Technology 9: 1373-1377; Hoogenboom et al. (1991) Nucl. Acid Res. 19: 4133-4137; and Barbas et al. (1991) Proc. Natl. Acad. Sci. USA 88: 7978-7982, and U.S. Patent Publication No. 2003/0186374.

Parent antibodies of the present invention can also be generated using various phage display methods known in the art. In phage display methods, functional antibody domains are displayed on the surface of phage particles which carry the polynucleotide sequences encoding them. In a particular, such phage can be utilized to display antigen-binding domains expressed from a repertoire or combinatorial antibody library (e. g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al. (1995) J. Immunol. Methods 182: 41-50; Ames et al. (1995) J. Immunol. Methods 184: 177-186; Kettleborough et al. (1994) Eur. J. Immunol. 24: 952-958; Persic et al. (1997) Gene 187: 9-18; Burton et al. (1994) Advances in Immunol. 57: 191-280; PCT Application No. PCT/GB91/01134; PCT Publication Nos. WO 90/02809; WO 91/10737; WO 92/01047; WO 92/18619; WO 93/11236; WO 95/15982; and WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

As described in the herein references, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to produce recombinantly Fab, Fab′ and F(ab′)2 fragments can also be employed using methods known in the art such as those disclosed in PCT Publication No. WO 92/22324; Mullinax et al. (1992) BioTechniques 12(6): 864-869; Sawai et al. (1995) AJRI 34: 26-34; and Better et al. (1988) Science 240: 1041-1043. Examples of techniques, which can be used to produce single-chain Fvs and antibodies, include those described in U.S. Pat. Nos. 4,946,778 and 5,258,498; Huston et al. (1991), Methods Enzymol. 203:46-88; Shu et al. (1993) Proc. Natl. Acad. Sci. USA 90: 7995-7999; and Skerra et al. (1988) Science 240: 1038-1040.

Alternative to screening of recombinant antibody libraries by phage display, other methodologies known in the art for screening large combinatorial libraries can be applied to the identification of parent antibodies. One type of alternative expression system is one in which the recombinant antibody library is expressed as RNA-protein fusions, as described in PCT Publication No. WO 98/31700, and in Roberts, R. W. and Szostak, J. W. (1997) Proc. Natl. Acad. Sci. USA 94:12297-12302. In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described herein (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described herein.

In another approach the parent antibodies can also be generated using yeast display methods known in the art. In yeast display methods, genetic methods are used to tether antibody domains to the yeast cell wall and display them on the surface of yeast. In particular, such yeast can be utilized to display antigen-binding domains expressed from a repertoire or combinatorial antibody library (e.g., human or murine). Examples of yeast display methods that can be used to make the parent antibodies include those disclosed in U.S. Pat. No. 6,699,658.

The antibodies described herein can be further modified to generate CDR grafted and humanized parent antibodies. CDR-grafted parent antibodies comprise heavy and light chain variable region sequences from a human antibody wherein one or more of the CDR regions of VH and/or VL are replaced with CDR sequences of murine antibodies that bind an antigen of interest. A framework sequence from any human antibody may serve as the template for CDR grafting. However, straight chain replacement onto such a framework often leads to some loss of binding affinity to the antigen. The more homologous a human antibody is to the original murine antibody, the less likely the possibility that combining the murine CDRs with the human framework will introduce distortions in the CDRs that could reduce affinity. Therefore, in an embodiment, the human variable framework that is chosen to replace the murine variable framework apart from the CDRs have at least a 65% sequence identity with the murine antibody variable region framework. In an embodiment, the human and murine variable regions apart from the CDRs have at least 70% sequence identify. In a particular embodiment, that the human and murine variable regions apart from the CDRs have at least 75% sequence identity. In another embodiment, the human and murine variable regions apart from the CDRs have at least 80% sequence identity. Methods for producing such antibodies are known in the art (see EP 239,400; PCT Publication No. WO 91/09967; and U.S. Pat. Nos. 5,225,539; 5,530,101; and 5,585,089), veneering or resurfacing (EP 592,106; EP 519,596; Padlan (1991) Mol. Immunol. 28(4/5): 489-498; Studnicka et al. (1994) Prot. Engineer. 7(6): 805-814; and Roguska et al. (1994) Proc. Acad. Sci. USA 91: 969-973), chain shuffling (U.S. Pat. No. 5,565,352), and anti-idiotypic antibodies.

Humanized antibodies are antibody molecules from non-human species antibody that binds the desired antigen having one or more complementarity determining regions (CDRs) from the non-human species and framework regions from a human immunoglobulin molecule. Known human Ig sequences are disclosed in, e.g., www._ncbi.nlm.nih.gov/entrez-/query.fcgi; xww._atcc.org/phage/hdb.html; www._sciquest.com/; www._abcam.com/; www._antibodyresource.com/onlinecomp.html; www._public.iastate.edu/.about.pedro/research_tools.html; www._mgen.uniheidelberg.de/SD/IT/IT.html; www._whfreeman.com/immunology/CH-05/kuby05.htm; www._library.thinkquest.org/12429/Immune/Antibody.html; www._hhmi.org/grants/lectures/1996/vlab/; www._path.cam.ac.uk/.about.mrc7/m-ikeimages.html; www._antibodyresource.com/; mcb.harvard.edu/BioLinks/Immunology.html; www._immunologylink.com/; pathbox.wustl.edu/.about.hcenter/index.-html; www._biotech.ufl.edu/.about.hcl/; www._pebio.com/pa/340913/340913. html-; www._nal.usda.gov/awic/pubs/antibody/; www._m.ehime-u.acjp/.about.yasuhito-/Elisa.html; www._biodesign.com/table.asp; www._icnet.uk/axp/facs/davies/lin-ks.html; www._biotech.ufl.edu/.about.fccl/protocol.html; www._isac-net.org/sites_geo.html; aximtl.imt.uni-marburg.de/.about.rek/AEP-Start.html; baserv.uci.kun.nl/.about.jraats/linksl.html; www._recab.uni-hd.de/immuno.bme.nwu.edu/; www._mrc-cpe.cam.ac.uk/imt-doc/pu-blic/INTRO.html; www._ibt.unam.mx/vir/V_mice.html; imgt.cnusc.fr:8104/; www._biochem.ucl.ac.uk/.about.martin/abs/index.html; antibody.bath.ac.uk/; abgen.cvm.tamu.edu/lab/wwwabgen.html; www._unizh.ch/.about.honegger/AHOseminar/Slide01.html; www._cryst.bbk.ac.uk/.about.ubcg07s/; www._nimr.mrc.ac.uk/CC/ccaewg/ccaewg.htm; www._path.cam.ac.uk/.about.mrc7/humanization/TAHHP. html; www._ibt.unam.mx/vir/structure/stat_aim.html; www._biosci.missouri.edu/smithgp/index.html; www._cryst.bioc.cam.ac.uk/abo-ut.fmolina/Web-pages/Pept/spottech.html; www._jerini.de/frroducts.htm; www._patents.ibm.com/ibm.html. Kabat et al, Sequences of Proteins of Immunological Interest, U.S. Dept. Health (1983). Such imported sequences can be used to reduce immunogenicity or reduce, enhance or modify binding, affinity, on-rate, off-rate, avidity, specificity, half-life, or any other suitable characteristic, as known in the art.

Framework residues in the human framework regions may be substituted with the corresponding residue from the CDR donor antibody to alter, e.g., improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., U.S. Pat. No. 5,585,089; Riechmann et al. (1988) Nature 332:323). Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding. Antibodies can be humanized using a variety of techniques known in the art, such as but not limited to those described in Jones et al. (1986) Nature 321: 522; Verhoeyen et al. (1988) Science 239: 1534; Sims et al. (1993) J. Immunol. 151: 2296; Chothia and Lesk (1987) J. Mol. Biol. 196: 901; Carter et al. (1992) Proc. Natl. Acad. Sci. USA 89: 4285; Presta et al. (1993) J. Immunol. 151: 2623; Padlan (1991) Mol. Immunol. 28(4/5): 489-498; Studnicka et al. (1994) Prot. Engineer. 7(6): 805-814; Roguska et al., (1994) Proc. Natl. Acad. Sci. USA 91: 969-973; PCT Publication No. WO 91/09967: U.S. Ser. Nos. 98/16,280; 96/18,978; 91/09,630; 91/05,939; 94/01234; GB89/01334; GB91/01134; GB92/01755; WO90/14443; WO90/14424; and WO90/14430; European Patent Publication Nos. EP 229246; EP 592,106; EP 519,596; and EP 239,400; and U.S. Pat. Nos. 5,565,332; 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

### B. Criteria for Selecting Parent Monoclonal Antibodies

A part of the invention pertains to selecting parent antibodies with at least one or more properties desired in the DVD-Ig molecule. The desired property is selected from one or more antibody parameters selected from the group consisting of antigen specificity, affinity to antigen, potency, biological function, epitope recognition, stability, solubility, production efficiency, immunogenicity, pharmacokinetics, bioavailability, tissue cross reactivity, and orthologous antigen binding.

### B1. Affinity to Antigen

The desired affinity of a therapeutic mAb may depend upon the nature of the antigen, and the desired therapeutic end-point. In an embodiment, monoclonal antibodies have higher affinities (Kd=0.01-0.50 pM) when blocking a cytokine-cytokine receptor interaction as such interaction are usually high affinity interactions (e.g., <pM-<nM ranges). In such instances, the mAb affinity for its target should be equal to or better than the affinity of the cytokine (ligand) for its receptor. On the other hand, mAb with lesser affinity (>nM range) could be therapeutically effective e.g., in clearing circulating potentially pathogenic proteins e.g., monoclonal antibodies that bind to, sequester, and clear circulating species of A-β amyloid. In other instances, reducing the affinity of an existing high affinity mAb by site-directed mutagenesis or using a mAb with lower affinity for its target could be used to avoid potential side-effects e.g., a high affinity mAb may sequester/neutralize all of its intended target, thereby completely depleting/eliminating the function(s) of the targeted protein. In this scenario, a low affinity mAb may sequester/neutralize a fraction of the target that may be responsible for the disease symptoms (the pathological or over-produced levels), thus allowing a fraction of the target to continue to perform its normal physiological function(s). Therefore, it may be possible to reduce the Kd to adjust dose and/or reduce side-effects. The affinity of the parental mAb might play a role in appropriately targeting cell surface molecules to achieve desired therapeutic out-come. For example, if a target is expressed on cancer cells with high density and on normal cells with low density, a lower affinity mAb will bind a greater number of targets on tumor cells than normal cells, resulting in tumor cell elimination via ADCC or CDC, and therefore might have therapeutically desirable effects. Thus selecting a mAb with desired affinity may be relevant for both soluble and surface targets.

Signaling through a receptor upon interaction with its ligand may depend upon the affinity of the receptor-ligand interaction. Similarly, it is conceivable that the affinity of a mAb for a surface receptor could determine the nature of intracellular signaling and whether the mAb may deliver an agonist or an antagonist signal. The affinity-based nature of mAb-mediated signaling may have an impact of its side-effect profile. Therefore, the desired affinity and desired functions of therapeutic monoclonal antibodies need to be determined carefully by in vitro and in vivo experimentation.

The desired Kd of a binding protein (e.g., an antibody) may be determined experimentally depending on the desired therapeutic outcome. In an embodiment parent antibodies with affinity (Kd) for a particular antigen equal to, or better than, the desired affinity of the DVD-Ig for the same antigen are selected. The parent antibodies for a given DVD-Ig molecule can be the same antibody or different antibodies. The antigen binding affinity and kinetics are assessed by Biacore or another similar technique. In one embodiment, each parent antibody has a dissociation constant (Kd) to its antigen selected from the group consisting of: at most about 10-7 M; at most about 10-8 M; at most about 10-9 M; at most about 10-10 M; at most about 10-11 M; at most about 10-12 M; and at most 10-13 M. The first parent antibody from which VD1 is obtained and the second parent antibody from which VD2 is obtained may have similar or different affinity (KD) for the respective antigen. Each parent antibody has an on rate constant (Kon) to the antigen selected from the group consisting of: at least about 102M-1s-1; at least about 103M-1s-1; at least about 104M-1s-1; at least about 105M-1s-1; and at least about 106M-1s-1, as measured by surface plasmon resonance. The first parent antibody from which VD1 is obtained and the second parent antibody from which VD2 is obtained may have similar or different on rate constant (Kon) for the respective antigen. In one embodiment, each parent antibody has an off rate constant (Koff) to the antigen selected from the group consisting of: at most about 10-3s-1; at most about 10-4s-1; at most about 10-5s-1; and at most about 10-6s-1, as measured by surface plasmon resonance. The first parent antibody from which VD1 is obtained and the second parent antibody from which VD2 is obtained may have similar or different off rate constants (Koff) for the respective antigen.

### B2. Potency

The desired affinity/potency of parental monoclonal antibodies will depend on the desired therapeutic outcome. For example, for receptor-ligand (R-L) interactions the affinity (kd) is equal to or better than the R-L kd (pM range). For simple clearance of a pathologic circulating protein, the kd could be in low nM range e.g., clearance of various species of circulating A-β peptide. In addition, the kd will also depend on whether the target expresses multiple copies of the same epitope e.g., a mAb targeting conformational epitope in Aβ oligomers.

Where VDI and VD2 bind the same antigen, but distinct epitopes, the DVD-Ig will contain four binding sites for the same antigen, thus increasing avidity and thereby the apparent kd of the DVD-Ig. In an embodiment, parent antibodies with equal or lower kd than that desired in the DVD-Ig are chosen. The affinity considerations of a parental mAb may also depend upon whether the DVD-Ig contains four or more identical antigen binding sites (i.e., a DVD-Ig from a single mAb). In this case, the apparent kd would be greater than the mAb due to avidity. Such DVD-Igs can be employed for cross-linking surface receptor, increase neutralization potency, enhance clearance of pathological proteins, etc.

In an embodiment parent antibodies with neutralization potency for specific antigen equal to or better than the desired neutralization potential of the DVD-Ig for the same antigen are selected. The neutralization potency can be assessed by a target-dependent bioassay where cells of appropriate type produce a measurable signal (i.e., proliferation or cytokine production) in response to target stimulation, and target neutralization by the mAb can reduce the signal in a dose-dependent manner.

### B3. Biological Functions

Monoclonal antibodies can perform potentially several functions. Some of these functions are listed in the Table immediately below. These functions can be assessed by both in vitro assays (e.g., cell-based and biochemical assays) and in vivo animal models.

TABLE
Some Potential Applications For Therapeutic Antibodies

| Target (Class) | Mechanism of Action (target) |
|---|---|
| Soluble | Neutralization of activity (e.g., a cytokine) |
| (cytokines, other) | Enhance clearance (e.g., Aβ oligomers) |
| | Increase half-life (e.g., GLP 1) |
| Cell Surface | Agonist (e.g., GLP1 R; EPO R; etc.) |
| (Receptors, other) | Antagonist (e.g., integrins; etc.) |
| | Cytotoxic (CD 20; etc.) |
| Protein deposits | Enhance clearance/degradation |
| | (e.g., Aβ plaques, amyloid deposits) |

MAbs with distinct functions described in the examples herein in the Table directly above can be selected to achieve desired therapeutic outcomes. Two or more selected parent monoclonal antibodies can then be used in DVD-Ig format to achieve two distinct functions in a single DVD-Ig molecule. For example, a DVD-Ig can be generated by selecting a parent mAb that neutralizes function of a specific cytokine, and selecting a parent mAb that enhances clearance of a pathological protein. Similarly, two parent monoclonal antibodies that recognize two different cell surface receptors can be selected, e.g., one mAb with an agonist function on one receptor and the other mAb with an antagonist function on a different receptor. These two selected monoclonal antibodies each with a distinct function can be used to construct a single DVD-Ig molecule that will possess the two distinct functions (agonist and antagonist) of the selected monoclonal antibodies in a single molecule. Similarly, two antagonistic monoclonal antibodies to cell surface receptors each blocking binding of respective receptor ligands (e.g., EGF and IGF) can be used in a DVD-Ig format. Conversely, an antagonistic anti-receptor mAb (e.g., anti-EGFR) and a neutralizing anti-soluble mediator (e.g., anti-IGF1/2) mAb can be selected to make a DVD-Ig.

### B4. Epitope Recognition

Different regions of proteins may perform different functions. For example specific regions of a cytokine interact with the cytokine receptor to bring about receptor activation whereas other regions of the protein may be required for stabilizing the cytokine. In this instance one may select a mAb that binds specifically to the receptor interacting region(s) on the cytokine and thereby blocks cytokine-receptor interaction. In some cases, for example, certain chemokine receptors that bind multiple ligands, a mAb that binds to the epitope (region on chemokine receptor) that interacts with only one ligand can be selected. In other instances, monoclonal antibodies can bind to epitopes on a target that are not directly responsible for physiological functions of the protein, but binding of a mAb to these regions could either interfere with physiological functions (steric hindrance) or alter the conformation of the protein such that the protein cannot function (mAb to receptors with multiple ligand which alter the receptor conformation such that none of the ligand can bind). Anti-cytokine monoclonal antibodies that do not block binding of the cytokine to its receptor, but block signal transduction have also been identified.

Examples of epitopes and mAb functions include, but are not limited to, blocking Receptor-Ligand (R-L) interaction (neutralizing mAb that binds R-interacting site); steric hindrance resulting in diminished or no R-binding. An Ab can bind the target at a site other than a receptor binding site, but still interfere with receptor binding and functions of the target by inducing conformational change and eliminating function (e.g., Xolair), e.g., binding to R but blocking signaling (125-2H).

In one instance of the invention, the parental mAb needs to target the appropriate epitope for maximum efficacy. Such epitope should be conserved in the DVD-Ig. The binding epitope of a mAb can be determined by several approaches, including co-crystallography, limited proteolysis of mAb-antigen complex plus mass spectrometric peptide mapping (Legros V. et al. (2000) Protein Sci. 9:1002-10), phage displayed peptide libraries (O'Connor, K. H. et al. (2005) J. Immunol. Methods. 299:21-35), as well as mutagenesis (Wu C. et al. (2003) J. Immunol. 170:5571-7).

### B5. Physicochemical and Pharmaceutical Properties

Therapeutic treatment with antibodies often requires administration of high doses, often several mg/kg (due to a low potency on a mass basis as a consequence of a typically large molecular weight). In order to accommodate patient compliance and to adequately address chronic disease therapies and outpatient treatment, subcutaneous (s.c.) or intramuscular (i.m.) administration of therapeutic mAbs is desirable. For example, the maximum desirable volume for s.c. administration is ^{˜}1.0 mL, and therefore, concentrations of >100 mg/mL are desirable to limit the number of injections per dose. The therapeutic antibody may be administered in one dose. The development of such formulations is constrained, however, by protein-protein interactions (e.g., aggregation, which potentially increases immunogenicity risks) and by limitations during processing and delivery (e.g., viscosity). Consequently, the large quantities required for clinical efficacy and the associated development constraints limit full exploitation of the potential of antibody formulation and s.c. administration in high-dose regimens. It is apparent that the physicochemical and pharmaceutical properties of a protein molecule and the protein solution are of utmost importance, e.g., stability, solubility and viscosity features.

### B5.1. Stability

A “stable” antibody formulation is one in which the antibody therein essentially retains its physical stability and/or chemical stability and/or biological activity upon storage. Stability can be measured at a selected temperature for a selected time period. In an embodiment, the antibody in the formulation is stable at room temperature (about 30° C.) or at 40° C. for at least 1 month and/or stable at about 2-8° C. for at least 1 year, such as, for at least 2 years. Furthermore, in an embodiment, the formulation is stable following freezing (to, e.g., −70° C.) and thawing of the formulation, hereinafter referred to as a “freeze/thaw cycle.” In another example, a “stable” formulation may be one wherein less than about 10% and less than about 5% of the protein is present as an aggregate in the formulation.

A DVD-Ig that is stable in vitro at various temperatures for an extended time period is desirable. One can achieve this by rapid screening of parental mAbs that are stable in vitro at elevated temperature, e.g., at 40° C. for 2-4 weeks, and then assess stability. During storage at 2-8° C., the protein reveals stability for at least 12 months, e.g., at least 24 months. Stability (% of monomeric, intact molecule) can be assessed using various techniques such as cation exchange chromatography, size exclusion chromatography, SDS-PAGE, as well as bioactivity testing. For a more comprehensive list of analytical techniques that may be employed to analyze covalent and conformational modifications see Jones, A. J. S. (1993) Analytical methods for the assessment of protein formulations and delivery systems. In: Cleland, J. L.; Langer, R., editors. Formulation and delivery of peptides and proteins, 1st edition, Washington, ACS, pg. 22-45; and Pearlman, R.; Nguyen, T. H. (1990) Analysis of protein drugs. In: Lee, V. H., editor. Peptide and protein drug delivery, 1st edition, New York, Marcel Dekker, Inc., pg. 247-301.

Heterogeneity and aggregate formation: stability of the antibody may be such that the formulation may reveal less than about 10%, such as less than about 5%, such as less than about 2%, or, within the range of 0.5% to 1.5% or less in the GMP antibody material that is present as aggregate. Size exclusion chromatography is a method that is sensitive, reproducible, and very robust in the detection of protein aggregates.

In addition to low aggregate levels, the antibody may in one instance, be chemically stable. Chemical stability may be determined by ion exchange chromatography (e.g., cation or anion exchange chromatography), hydrophobic interaction chromatography, or other methods such as isoelectric focusing or capillary electrophoresis. For instance, chemical stability of the antibody may be such that after storage of at least 12 months at 2-8° C. the peak representing unmodified antibody in a cation exchange chromatography may increase not more than 20%, such as not more than 10%, or not more than 5% as compared to the antibody solution prior to storage testing.

In an embodiment, the parent antibodies display structural integrity; correct disulfide bond formation, and correct folding: Chemical instability due to changes in secondary or tertiary structure of an antibody may impact antibody activity. For instance, stability as indicated by activity of the antibody may be such that after storage of at least 12 months at 2-8° C. the activity of the antibody may decrease not more than 50%, such as not more than 30%, not more than 10%, or not more than 5% or 1% as compared to the antibody solution prior to storage testing. Suitable antigen-binding assays can be employed to determine antibody activity.

### B5.2. Solubility

The “solubility” of a mAb correlates with the production of correctly folded, monomeric IgG. The solubility of the IgG may therefore be assessed by HPLC. For example, soluble (monomeric) IgG will give rise to a single peak on the HPLC chromatograph, whereas insoluble (e.g., multimeric and aggregated) will give rise to a plurality of peaks. A person skilled in the art will therefore be able to detect an increase or decrease in solubility of an IgG using routine HPLC techniques. For a more comprehensive list of analytical techniques that may be employed to analyze solubility (see Jones, A. G. (1993) Dep. Chem. Biochem. Eng., Univ. Coll. London, London, UK. Editor(s): Shamlou, P. Ayazi. Process. Solid-Liq. Suspensions, 93-117. Publisher: Butterworth-Heinemann, Oxford, UK and Pearlman et al. (1990) Adv. in Parenteral Sci. 4 (Pept. Protein Drug Delivery): 247-301). Solubility of a therapeutic mAb is critical for formulating to high concentration often required for adequate dosing. As outlined herein, solubilities of >100 mg/mL may be required to accommodate efficient antibody dosing. For instance, antibody solubility may be not less than about 5 mg/mL in early research phase, not less than about 25 mg/mL in advanced process science stages, or not less than about 100 mg/mL, or not less than about 150 mg/mL. It is obvious to a person skilled in the art that the intrinsic properties of a protein molecule are important the physico-chemical properties of the protein solution, e.g., stability, solubility, viscosity. However, a person skilled in the art will appreciate that a broad variety of excipients exist that may be used as additives to beneficially impact the characteristics of the final protein formulation. These excipients may include: (i) liquid solvents, cosolvents (e.g., alcohols such as ethanol); (ii) buffering agents (e.g., phosphate, acetate, citrate, and amino acid buffers); (iii) sugars or sugar alcohols (e.g., sucrose, trehalose, fructose, raffinose, mannitol, sorbitol, and dextrans); (iv) surfactants (e.g., polysorbate 20, 40, 60, 80, and poloxamers); (v) isotonicity modifiers (e.g., salts such as NaCl, sugars, and sugar alcohols); and (vi) others (e.g., preservatives, chelating agents, antioxidants, chelating substances (e.g., EDTA), biodegradable polymers, and carrier molecules (e.g., HSA and PEGS)).

Viscosity is a parameter of high importance with regard to antibody manufacture and antibody processing (e.g., diafiltration/ultrafiltration), fill-finish processes (pumping aspects, filtration aspects) and delivery aspects (syringeability, sophisticated device delivery). Low viscosities enable the liquid solution of the antibody having a higher concentration. This enables the same dose may be administered in smaller volumes. Small injection volumes inhere the advantage of lower pain on injection sensations, and the solutions not necessarily have to be isotonic to reduce pain on injection in the patient. The viscosity of the antibody solution may be such that at shear rates of 100 (1/s) antibody solution viscosity is below 200 mPa s, below 125 mPa s, below 70 mPa s, and below 25 mPa s, or even below 10 mPa s.

### B5.3. Production Efficiency

The generation of a DVD-Ig that is efficiently expressed in mammalian cells, such as Chinese hamster ovary cells (CHO), will in an embodiment require two parental monoclonal antibodies which are themselves expressed efficiently in mammalian cells. The production yield from a stable mammalian line (i.e. CHO) should be above about 0.5 g/L, above about 1 g/L, or in the range of about 2 to about 5 g/L or more (Kipriyanov, S. M. and Little, M. (1999) Mol. Biotechnol. 12: 173-201; Carroll, S. and Al-Rubeai, M. (2004) Expert Opin. Biol. Ther. 4: 1821-9).

Production of antibodies and Ig fusion proteins in mammalian cells is influenced by several factors. Engineering of the expression vector via incorporation of strong promoters, enhancers and selection markers can maximize transcription of the gene of interest from an integrated vector copy. The identification of vector integration sites that are permissive for high levels of gene transcription can augment protein expression from a vector (Wurm et al. (2004) Nature Biotechnol. 22(11): 1393-1398). Furthermore, levels of production are affected by the ratio of antibody heavy and light chains and various steps in the process of protein assembly and secretion (Jiang et al. (2006) Biotechnol. Prog. 22(1): 313-8).

### B6. Immunogenicity

Administration of a therapeutic mAb may result in certain incidence of an immune response (i.e., the formation of endogenous antibodies directed against the therapeutic mAb). Potential elements that might induce immunogenicity should be analyzed during selection of the parental monoclonal antibodies, and steps to reduce such risk can be taken to optimize the parental monoclonal antibodies prior to DVD-Ig construction. Mouse-derived antibodies have been found to be highly immunogenic in patients. The generation of chimeric antibodies comprised of mouse variable and human constant regions presents a logical next step to reduce the immunogenicity of therapeutic antibodies (Morrison and Schlom, 1990). Alternatively, immunogenicity can be reduced by transferring murine CDR sequences into a human antibody framework (reshaping/CDR grafting/humanization), as described for a therapeutic antibody by Riechmann et al. (1988) Nature 332: 323-327. Another method is referred to as “resurfacing” or “veneering,” starting with the rodent variable light and heavy domains, only surface-accessible framework amino acids are altered to human ones, while the CDR and buried amino acids remain from the parental rodent antibody (Roguska et al. (1996) Prot. Engineer 9: 895-904). In another type of humanization, instead of grafting the entire CDRs, one technique grafts only the “specificity-determining regions” (SDRs), defined as the subset of CDR residues that are involved in binding of the antibody to its target (Kashmiri et al. (2005) Methods 36(1): 25-34). This necessitates identification of the SDRs either through analysis of available three-dimensional structures of antibody-target complexes or mutational analysis of the antibody CDR residues to determine which interact with the target. Alternatively, fully human antibodies may have reduced immunogenicity compared to murine, chimeric or humanized antibodies.

Another approach to reduce the immunogenicity of therapeutic antibodies is the elimination of certain specific sequences that are predicted to be immunogenic. In one approach, after a first generation biologic has been tested in humans and found to be unacceptably immunogenic, the B-cell epitopes can be mapped and then altered to avoid immune detection. Another approach uses methods to predict and remove potential T-cell epitopes. Computational methods have been developed to scan and to identify the peptide sequences of biologic therapeutics with the potential to bind to MHC proteins (Desmet et al. (2005) Proteins 58: 53-69). Alternatively a human dendritic cell-based method can be used to identify CD4+ T-cell epitopes in potential protein allergens (Stickler et al. (2000) J. Immunother. 23: 654-60; S. L. Morrison and J. Schlom, (1990) Important Adv. Oncol. 3-18; Riechmann et al. (1988) Nature 332: 323-327; Roguska et al. (1996) Protein Engineer. 9: 895-904; Kashmiri et al. (2005) Methods 36(1): 25-34; Desmet et al. (2005) Proteins 58: 53-69; and Stickler et al. (2000) J. Immunotherapy 23: 654-60.)

### B7. In Vivo Efficacy

To generate a DVD-Ig molecule with desired in vivo efficacy, it is important to generate and select mAbs with similarly desired in vivo efficacy when given in combination. However, in some instances the DVD-Ig may exhibit in vivo efficacy that cannot be achieved with the combination of two separate mAbs. For instance, a DVD-Ig may bring two targets in close proximity leading to an activity that cannot be achieved with the combination of two separate mAbs. Additional desirable biological functions are described herein in section B 3. Parent antibodies with characteristics desirable in the DVD-Ig molecule may be selected based on factors such as pharmacokinetic t 1/2; tissue distribution; soluble versus cell surface targets; and target concentration-soluble/density-surface.

### B8. In Vivo Tissue Distribution

To generate a DVD-Ig molecule with desired in vivo tissue distribution, in an embodiment parent mAbs with similar desired in vivo tissue distribution profile must be selected. In this regard, the parent mAbs can be the same antibody or different antibodies. Alternatively, based on the mechanism of the dual-specific targeting strategy, it may at other times not be required to select parent mAbs with the similarly desired in vivo tissue distribution when given in combination. (e.g., in the case of a DVD-Ig in which one binding component targets the DVD-Ig to a specific site thereby bringing the second binding component to the same target site). For example, one binding specificity of a DVD-Ig could target pancreas (islet cells) and the other specificity could bring GLP1 to the pancreas to induce insulin.

### B 9. Isotype

To generate a DVD-Ig molecule with desired properties including, but not limited to, isotype, effector functions, and the circulating half-life, in an embodiment parent mAbs with appropriate Fc-effector functions depending on the therapeutic utility and the desired therapeutic end-point are selected. The parent mAbs can be the same antibody or different antibodies. There are five main heavy-chain classes or isotypes some of which have several sub-types and these determine the effector functions of an antibody molecule. These effector functions reside in the hinge region, CH2 and CH3 domains of the antibody molecule. However, residues in other parts of an antibody molecule may have effects on effector functions as well. The hinge region Fc-effector functions include: (i) antibody-dependent cellular cytotoxicity, (ii) complement (C1q) binding, activation and complement-dependent cytotoxicity (CDC), (iii) phagocytosis/clearance of antigen-antibody complexes, and (iv) cytokine release in some instances. These Fc-effector functions of an antibody molecule are mediated through the interaction of the Fc-region with a set of class-specific cell surface receptors. Antibodies of the IgG1 isotype are most active while IgG2 and IgG4 having minimal or no effector functions. The effector functions of the IgG antibodies are mediated through interactions with three structurally homologous cellular Fc receptor types (and sub-types) (FcgR1, FcgRII and FcgRIII). These effector functions of an IgG1 can be eliminated by mutating specific amino acid residues in the lower hinge region (e.g., L234A, L235A) that are required for FcgR and C1q binding Amino acid residues in the Fc region, in particular the CH2-CH3 domains, also determine the circulating half-life of the antibody molecule. This Fc function is mediated through the binding of the Fc-region to the neonatal Fc receptor (FcRn), which is responsible for recycling of antibody molecules from the acidic lysosomes back to the general circulation.

Whether a mAb should have an active or an inactive isotype will depend on the desired therapeutic end-point for an antibody. Some examples of usage of isotypes and desired therapeutic outcome are listed below:

a) If the desired end-point is functional neutralization of a soluble cytokine, then an inactive isotype may be used;
b) If the desired out-come is clearance of a pathological protein, an active isotype may be used;
c) If the desired outcome is clearance of protein aggregates, an active isotype may be used;
d) If the desired outcome is to antagonize a surface receptor, an inactive isotype is used (Tysabri, IgG4; OKT3, mutated IgG1);
e) If the desired outcome is to eliminate target cells, an active isotype is used (Herceptin, IgG1 (and with enhanced effector functions); and
f) If the desired outcome is to clear proteins from circulation without entering the CNS, an IgM isotype may be used (e.g., clearing circulating Ab peptide species).

The Fc effector functions of a parental mAb can be determined by various in vitro methods well known in the art.

As discussed, the selection of isotype, and thereby the effector functions will depend upon the desired therapeutic end-point. In cases where simple neutralization of a circulating target is desired, for example blocking receptor-ligand interactions, the effector functions may not be required. In such instances isotypes or mutations in the Fc-region of an antibody that eliminate effector functions are desirable. In other instances where elimination of target cells is the therapeutic end-point, for example elimination of tumor cells, isotypes or mutations or de-fucosylation in the Fc-region that enhance effector functions are desirable (Presta, G. L. (2006) Adv. Drug Deliv. Rev. 58:640-656 and Satoh, M. et al. (2006) Expert Opin. Biol. Ther. 6: 1161-1173). Similarly, depending up on the therapeutic utility, the circulating half-life of an antibody molecule can be reduced/prolonged by modulating antibody-FcRn interactions by introducing specific mutations in the Fc region (Dall'Acqua, W. F. et al. (2006) J. Biol. Chem. 281: 23514-23524; Petkova, S. B. (2006) et al., Internat. Immunol. 18:1759-1769; Vaccaro, C. et al. (2007) Proc. Natl. Acad. Sci. USA 103: 18709-18714).

The published information on the various residues that influence the different effector functions of a normal therapeutic mAb may need to be confirmed for DVD-Ig. It may be possible that in a DVD-Ig format additional (different) Fc-region residues, other than those identified for the modulation of monoclonal antibody effector functions, may be important.

Overall, the decision as to which Fc-effector functions (isotype) will be critical in the final DVD-Ig format will depend upon the disease indication, therapeutic target, and desired therapeutic end-point and safety considerations. Listed below are exemplary appropriate heavy chain and light chain constant regions including, but not limited to: IgG1-allotype: Glmz, IgG1 mutant-A234, A235, IgG2-allotype: G2m(n−), Kappa-Km3, Lambda.

Fc Receptor and C1q Studies: The possibility of unwanted antibody-dependent cell-mediated cytotoxicity (ADCC) and complement-dependent cytotoxicity (CDC) by antibody complexing to any overexpressed target on cell membranes can be abrogated by the (for example, L234A, L235A) hinge-region mutations. These substituted amino acids, present in the IgG1 hinge region of mAb, are expected to result in diminished binding of mAb to human Fc receptors (but not FcRn), as FcgR binding is thought to occur within overlapping sites on the IgG1 hinge region. This feature of mAb may lead to an improved safety profile over antibodies containing a wild-type IgG. Binding of mAb to human Fc receptors can be determined by flow cytometry experiments using cell lines (e.g., THP-1, K562) and an engineered CHO cell line that expresses FcgRIIb (or other FcgRs). Compared to IgG1 control monoclonal antibodies, mAb show reduced binding to FcgRI and FcgRIIa, whereas binding to FcgRIIb is unaffected. The binding and activation of C1q by antigen/IgG immune complexes triggers the classical complement cascade with consequent inflammatory and/or immunoregulatory responses. The C1q binding site on IgGs has been localized to residues within the IgG hinge region. C1q binding to increasing concentrations of mAb was assessed by C1q ELISA. The results demonstrate that mAb is unable to bind to C1q, as expected when compared to the binding of a wildtype control IgG1. Overall, the L234A, L235A hinge region mutation abolishes binding of mAb to FcgRI, FcgRIIa and C1q but does not impact the interaction of mAb with FcgRIIb. These data suggest that in vivo, mAb with mutant Fc will interact normally with the inhibitory FcgRIIb but will likely fail to interact with the activating FcgRI and FcgRIIa receptors or C1q.

Human FcRn binding: The neonatal receptor (FcRn) is responsible for transport of IgG across the placenta and to control the catabolic half-life of the IgG molecules. It might be desirable to increase the terminal half-life of an antibody to improve efficacy, to reduce the dose or frequency of administration, or to improve localization to the target. Alternatively, it might be advantageous to do the converse that is, to decrease the terminal half-life of an antibody to reduce whole body exposure or to improve the target-to-non-target binding ratios. Tailoring the interaction between IgG and its salvage receptor, FcRn, offers a way to increase or decrease the terminal half-life of IgG. Proteins in the circulation, including IgG, are taken up in the fluid phase through micropinocytosis by certain cells, such as those of the vascular endothelia. IgG can bind FcRn in endosomes under slightly acidic conditions (pH 6.0-6.5) and can recycle to the cell surface, where it is released under almost neutral conditions (pH 7.0-7.4). Mapping of the Fc-region-binding site on FcRn80, 16, 17 showed that two histidine residues that are conserved across species, His310 and His435, are responsible for the pH dependence of this interaction. Using phage-display technology, a mouse Fc-region mutation that increases binding to FcRn and extends the half-life of mouse IgG was identified (see Victor, G. et al. (1997) Nature Biotechnol. 15(7): 637-640). Fc-region mutations that increase the binding affinity of human IgG for FcRn at pH 6.0, but not at pH 7.4, have also been identified (see Dall'Acqua, William F., et al. (2002) J. Immunol. 169(9): 5171-80). Moreover, in one case, a similar pH-dependent increase in binding (up to 27-fold) was also observed for rhesus FcRn, and this resulted in a twofold increase in serum half-life in rhesus monkeys compared with the parent IgG (see Hinton, P. R. et al. (2004) J. Biol. Chem. 279(8), 6213-6216). These findings indicate that it is feasible to extend the plasma half-life of antibody therapeutics by tailoring the interaction of the Fc region with FcRn. Conversely, Fc-region mutations that attenuate interaction with FcRn can reduce antibody half-life.

### B.10 Pharmacokinetics (PK)

To generate a DVD-Ig molecule with desired pharmacokinetic profile, in an embodiment parent mAbs with the similarly desired pharmacokinetic profile are selected. One consideration is that immunogenic response to monoclonal antibodies (i.e., HAHA, human anti-human antibody response; HACA, human anti-chimeric antibody response) further complicates the pharmacokinetics of these therapeutic agents. In an embodiment, monoclonal antibodies with minimal or no immunogenicity are used for constructing DVD-Ig molecules such that the resulting DVD-Igs will also have minimal or no immunogenicity. Some of the factors that determine the PK of a mAb include, but are not limited to, intrinsic properties of the mAb (VH amino acid sequence); immunogenicity; FcRn binding and Fc functions.

The PK profile of selected parental monoclonal antibodies can be easily determined in rodents as the PK profile in rodents correlates well with (or closely predicts) the PK profile of monoclonal antibodies in cynomolgus monkey and humans. The PK profile is determined by any means known to a person of ordinary skill in the art.

After the parental monoclonal antibodies with desired PK characteristics (and other desired functional properties as discussed herein) are selected, the DVD-Ig is constructed. As the DVD-Ig molecules contain two antigen-binding domains from two parental monoclonal antibodies, the PK properties of the DVD-Ig are assessed as well. Therefore, while determining the PK properties of the DVD-Ig, PK assays may be employed that determine the PK profile based on functionality of both antigen-binding domains derived from the two parent monoclonal antibodies. The PK profile of a DVD-Ig can be determined by any means known to a person of ordinary skill in the art. Additional factors that may impact the PK profile of DVD-Ig include the antigen-binding domain (CDR) orientation; linker size; and Fc/FcRn interactions. PK characteristics of parent antibodies can be evaluated by assessing the following parameters: absorption, distribution, metabolism and excretion.

Absorption: administration of therapeutic monoclonal antibodies is most often via parenteral routes (e.g., intravenous [IV], subcutaneous [SC], or intramuscular [IM]). Absorption of a mAb into the systemic circulation following either SC or IM administration from the interstitial space is primarily through the lymphatic pathway. Saturable, presystemic, proteolytic degradation may result in variable absolute bioavailability following extravascular administration. Usually, increases in absolute bioavailability with increasing doses of monoclonal antibodies may be observed due to saturated proteolytic capacity at higher doses. The absorption process for a mAb is usually quite slow as the lymph fluid drains slowly into the vascular system, and the duration of absorption may occur over hours to several days. The absolute bioavailability of monoclonal antibodies following SC administration generally ranges from 50% to 100%. In the case of a transport-mediating structure at the blood-brain barrier targeted by the DVD-Ig construct, circulation times in plasma may be reduced due to enhanced trans-cellular transport at the blood brain barrier (BBB) into the CNS compartment, where the DVD-Ig is liberated to enable interaction via its second antigen recognition site.

Distribution: Following IV administration, monoclonal antibodies usually follow a biphasic serum (or plasma) concentration-time profile, beginning with a rapid distribution phase, followed by a slow elimination phase. In general, a biexponential pharmacokinetic model best describes this kind of pharmacokinetic profile. The volume of distribution in the central compartment (Vc) for a mAb is usually equal to or slightly larger than the plasma volume (2-3 liters). A distinct biphasic pattern in serum (plasma) concentration versus time profile may not be apparent with other parenteral routes of administration, such as IM or SC, because the distribution phase of the serum (plasma) concentration-time curve is masked by the long absorption portion. Many factors, including physicochemical properties, site-specific and target-oriented receptor mediated uptake, binding capacity of tissue, and mAb dose can influence biodistribution of a mAb. Some of these factors can contribute to nonlinearity in biodistribution for a mAb.

Metabolism and Excretion: Due to the molecular size, intact monoclonal antibodies are not excreted into the urine via kidney. They are primarily inactivated by metabolism (e.g., catabolism). For IgG-based therapeutic monoclonal antibodies, half-lives typically ranges from hours or 1-2 days to over 20 days. The elimination of a mAb can be affected by many factors, including, but not limited to, affinity for the FcRn receptor, immunogenicity of the mAb, the degree of glycosylation of the mAb, the susceptibility for the mAb to proteolysis, and receptor-mediated elimination.

### B.11 Tissue Cross-Reactivity Pattern on Human and Tox Species

Identical staining pattern suggests that potential human toxicity can be evaluated in tox species. Tox species are those animal in which unrelated toxicity is studied.

The individual antibodies are selected to meet two criteria: (1) tissue staining appropriate for the known expression of the antibody target; and (2) similar staining pattern between human and tox species tissues from the same organ.

Criterion 1: Immunizations and/or antibody selections typically employ recombinant or synthesized antigens (proteins, carbohydrates or other molecules). Binding to the natural counterpart and counterscreen against unrelated antigens are often part of the screening funnel for therapeutic antibodies. However, screening against a multitude of antigens is often unpractical. Therefore tissue cross-reactivity studies with human tissues from all major organs serve to rule out unwanted binding of the antibody to any unrelated antigens.

Criterion 2: Comparative tissue cross reactivity studies with human and tox species tissues (cynomolgus monkey, dog, possibly rodents and others, the same 36 or 37 tissues are being tested as in the human study) help to validate the selection of a tox species. In the typical tissue cross-reactivity studies on frozen tissue sections therapeutic antibodies may demonstrate the expected binding to the known antigen and/or to a lesser degree binding to tissues based either on low level interactions (unspecific binding, low level binding to similar antigens, low level charge based interactions etc.). In any case the most relevant toxicology animal species is the one with the highest degree of coincidence of binding to human and animal tissue.

Tissue cross reactivity studies follow the appropriate regulatory guidelines including EC CPMP Guideline 111/5271/94 “Production and quality control of mAbs” and the 1997 U.S. FDA/CBER “Points to Consider in the Manufacture and Testing of Monoclonal Antibody Products for Human Use”. Cryosections (5 im) of human tissues obtained at autopsy or biopsy were fixed and dried on object glass. The peroxidase staining of tissue sections was performed, using the avidin-biotin system. FDA's Guidance “Points to Consider in the Manufacture and Testing of Monoclonal Antibody Products for Human Use”.

Tissue cross reactivity studies are often done in two stages, with the first stage including cryosections of 32 tissues (typically: Adrenal Gland, Gastrointestinal Tract, Prostate, Bladder, Heart, Skeletal Muscle, Blood Cells, Kidney, Skin, Bone Marrow, Liver, Spinal Cord, Breast, Lung, Spleen, Cerebellum, Lymph Node, Testes, Cerebral Cortex, Ovary, Thymus, Colon, Pancreas, Thyroid, Endothelium, Parathyroid, Ureter, Eye, Pituitary, Uterus, Fallopian Tube and Placenta) from one human donor. In the second phase a full cross reactivity study is performed with up to 38 tissues (including adrenal, blood, blood vessel, bone marrow, cerebellum, cerebrum, cervix, esophagus, eye, heart, kidney, large intestine, liver, lung, lymph node, breast mammary gland, ovary, oviduct, pancreas, parathyroid, peripheral nerve, pituitary, placenta, prostate, salivary gland, skin, small intestine, spinal cord, spleen, stomach, striated muscle, testis, thymus, thyroid, tonsil, ureter, urinary bladder, and uterus) from 3 unrelated adults. Studies are done typically at minimally two dose levels.

The therapeutic antibody (i.e., test article) and isotype matched control antibody may be biotinylated for avidin-biotin complex (ABC) detection; other detection methods may include tertiary antibody detection for a FITC (or otherwise) labeled test article, or precomplexing with a labeled anti-human IgG for an unlabeled test article.

Briefly, cryosections (about 5 μm) of human tissues obtained at autopsy or biopsy are fixed and dried on object glass. The peroxidase staining of tissue sections is performed, using the avidin-biotin system. First (in case of a precomplexing detection system), the test article is incubated with the secondary biotinylated anti-human IgG and developed into immune complex. The immune complex at the final concentrations of 2 and 10 pg/mL of test article is added onto tissue sections on object glass and then the tissue sections are reacted for 30 minutes with an avidin-biotin-peroxidase kit. Subsequently, DAB (3,3′-diaminobenzidine), a substrate for the peroxidase reaction, is applied for 4 minutes for tissue staining. Antigen-Sepharose beads are used as positive control tissue sections.

Any specific staining is judged to be either an expected (e.g., consistent with antigen expression) or unexpected reactivity based upon known expression of the target antigen in question. Any staining judged specific is scored for intensity and frequency. Antigen or serum competion or blocking studies can assist further in determining whether observed staining is specific or nonspecific.

If two selected antibodies are found to meet the selection criteria—appropriate tissue staining, and matching staining between human and toxicology animal specific tissue—they can be selected for DVD-Ig generation.

The tissue cross reactivity study has to be repeated with the final DVD-Ig construct, but while these studies follow the same protocol as outline herein, they are more complex to evaluate because any binding can come from any of the two parent antibodies, and any unexplained binding needs to be confirmed with complex antigen competition studies.

It is readily apparent that the complex undertaking of tissue crossreactivity studies with a multispecific molecule like a DVD-Ig is greatly simplified if the two parental antibodies are selected for (1) lack of unexpected tissue cross reactivity findings and (2) for appropriate similarity of tissue cross reactivity findings between the corresponding human and toxicology animal species tissues.

### B.12 Specificity and Selectivity

To generate a DVD-Ig molecule with desired specificity and selectivity, one needs to generate and select parent mAbs with the similarly desired specificity and selectivity profile. In this regard, parent mAbs can be the same antibody or different antibodies.

Binding studies for specificity and selectivity with a DVD-Ig can be complex due to the four or more binding sites, two each for each antigen. Briefly, binding studies using an enzyme linked immunosorbent assay (ELISA), BIAcore, KinExA, or other interaction studies with a DVD-Ig need to monitor the binding of one, two or more antigens to the DVD-Ig molecule. While BIAcore technology can resolve the sequential, independent binding of multiple antigens, more traditional methods, including ELISA, or more modern techniques, such as KinExA, cannot. Therefore, careful characterization of each parent antibody is critical. After each individual antibody has been characterized for specificity, confirmation of specificity retention of the individual binding sites in the DVD-Ig molecule is greatly simplified.

It is readily apparent that the complex undertaking of determining the specificity of a DVD-Ig is greatly simplified if the two parental antibodies are selected for specificity prior to being combined into a DVD-Ig.

Antigen-antibody interaction studies can take many forms, including many classical protein protein interaction studies, ELISA, mass spectrometry, chemical cross-linking, SEC with light scattering, equilibrium dialysis, gel permeation, ultrafiltration, gel chromatography, large-zone analytical SEC, micropreparative ultracentrifugation (sedimentation equilibrium), spectroscopic methods, titration microcalorimetry, sedimentation equilibrium (in analytical ultracentrifuge), sedimentation velocity (in analytical centrifuge), and surface plasmon resonance (including BIAcore). Relevant references include “Current Protocols in Protein Science,” Coligan, J. E. et al. (eds.) Volume 3, chapters 19 and 20, published by John Wiley & Sons Inc., and “Current Protocols in Immunology,” Coligan, J. E. et al. (eds.) published by John Wiley & Sons Inc., and relevant references included therein.

Cytokine Release in Whole Blood: The interaction of mAb with human blood cells can be investigated by a cytokine release (Wing, M. G. (1995) Therapeut. Immunol. 2(4): 183-190; “Current Protocols in Pharmacology,” Enna, S. J. et al. (eds.) published by John Wiley & Sons Inc; Madhusudan, S. (2004) Clin. Cancer Res. 10(19): 6528-6534; Cox, J. (2006) Methods 38(4): 274-282; Choi, I. (2001) Eur. J. Immunol. 31(1): 94-106). Briefly, various concentrations of mAb are incubated with human whole blood for 24 hours. The concentration tested should cover a wide range including final concentrations mimicking typical blood levels in patients (including but not limited to 100 ng/ml-100 μg/ml). Following the incubation, supernatants and cell lysates were analyzed for the presence of IL-1Rα, TNF, IL-1b, IL-6 and IL-8. Cytokine concentration profiles generated for mAb were compared to profiles produced by a negative human IgG control and a positive LPS or PHA control. The cytokine profile displayed by mAb from both cell supernatants and cell lysates was comparable to control human IgG. In an embodiment, the monoclonal antibody does not interact with human blood cells to spontaneously release inflammatory cytokines.

Cytokine release studies for a DVD-Ig are complex due to the four or more binding sites, two each for each antigen. Briefly, cytokine release studies as described herein measure the effect of the whole DVD-Ig molecule on whole blood or other cell systems, but can resolve which portion of the molecule causes cytokine release. Once cytokine release has been detected, the purity of the DVD-Ig preparation has to be ascertained, because some co-purifying cellular components can cause cytokine release on their own. If purity is not the issue, fragmentation of DVD-Ig (including but not limited to removal of Fc portion, separation of binding sites etc.), binding site mutagenesis or other methods may need to be employed to deconvolute any observations. It is readily apparent that this complex undertaking is greatly simplified if the two parental antibodies are selected for lack of cytokine release prior to being combined into a DVD-Ig.

### B.13 Cross Reactivity to Other Species for Toxicological Studies

In an embodiment, the individual antibodies are selected with sufficient cross-reactivity to appropriate tox species, for example, cynomolgus monkey. Parental antibodies need to bind to orthologous species target (i.e., cynomolgus monkey) and elicit appropriate response (modulation, neutralization, activation). In an embodiment, the cross-reactivity (affinity/potency) to orthologous species target should be within 10-fold of the human target. In practice, the parental antibodies are evaluated for multiple species, including mouse, rat, dog, monkey (and other non-human primates), as well as disease model species. The acceptable cross-reactivity to tox species from the parental monoclonal antibodies allows future toxicology studies of DVD-Ig-Ig in the same species. For that reason, the two parental monoclonal antibodies should have acceptable cross-reactivity for a common tox species, thereby allowing toxicology studies of DVD-Ig in the same species.

Parent mAbs may be selected from various mAbs that bind TNF (see for example, U.S. Pat. No. 6,258,562) and 11-33 or other specific targets disclosed herein.

### C. Construction of DVD Molecules

The dual variable domain immunoglobulin (DVD-Ig) molecule is designed such that two different light chain variable domains (VL) from the two different parent monoclonal antibodies are linked in tandem directly or via a short linker by recombinant DNA techniques, followed by the light chain constant domain, and optionally, an Fc region. Similarly, the heavy chain comprises two different heavy chain variable domains (VH) linked in tandem, followed by the constant domain CH1 and Fc region (FIG. 11).

The variable domains can be obtained using recombinant DNA techniques from a parent antibody generated by any one of the methods described herein. The variable domain may be a murine heavy or light chain variable domain, a CDR a human heavy or light chain variable domain.

The first and second variable domains may be linked directly to each other using recombinant DNA techniques, linked via a linker sequence, or the two variable domains are linked. The variable domains may bind the same antigen or may bind different antigens. DVD-Ig molecules of the invention may include one immunoglobulin variable domain and one non-immunoglobulin variable domain, such as ligand binding domain of a receptor, or an active domain of an enzyme. DVD-Ig molecules may also comprise two or more non-Ig domains.

The linker sequence may be a single amino acid or a polypeptide sequence such as the linker sequences listed herein. The choice of linker sequences is based on crystal structure analysis of several Fab molecules. There is a natural flexible linkage between the variable domain and the CH1/CL constant domain in Fab or antibody molecular structure. This natural linkage comprises approximately 10-12 amino acid residues, contributed by 4-6 residues from C-terminus of V domain and 4-6 residues from the N-terminus of CL/CH1 domain. DVD Igs of the invention were generated using N-terminal 5-6 amino acid residues, or 11-12 amino acid residues, of CL or CH1 as linker in light chain and heavy chain of DVD-Ig, respectively. The N-terminal residues of the CL or CH1 domain, particularly the first 5-6 amino acid residues, adopt a loop conformation without strong secondary structure, and, therefore, can act as a flexible linker between the two variable domains. The N-terminal residues of the CL or CH1 domain are a natural extension of the variable domains, as they are part of the Ig sequences, and, therefore, minimize to a large extent any immunogenicity potentially arising from the linkers and junctions.

Other linker sequences may include any sequence of any length of the CL/CH1 domain but not all residues of the CL/CH1 domain (for example, the first 5-12 amino acid residues of the CL/CH1 domains) the light chain linkers can be from Ck or Cl; and the heavy chain linkers can be derived from CH1 of any isotypes, including Cg1, Cg2, Cg3, Cg4, Ca1, Ca2, Cd, Ce, and Cm. Linker sequences may also be derived from other proteins such as Ig-like proteins, (e.g. TCR, FcR, KIR); G/S based sequences (e.g., G4S repeats)); hinge region-derived sequences; and other natural sequences from other proteins.

The constant domain may be linked to the two linked variable domains using recombinant DNA techniques. Sequence comprising linked heavy chain variable domains may be linked to a heavy chain constant domain and sequence comprising linked light chain variable domains is linked to a light chain constant domain. The constant domains may also be human heavy chain constant domain and human light chain constant domain respectively. The DVD heavy chain may be further linked to an Fc region. The Fc region may be a native sequence Fc region, or a variant Fc region, or a human Fc region, or a Fc region from IgG1, IgG2, IgG3, IgG4, IgA, IgM, IgE, or IgD.

Two heavy chain DVD polypeptides and two light chain DVD polypeptides may be combined to form a DVD-Ig molecule.

### D. Production of DVD Proteins

Binding proteins of the present invention may be produced by any of a number of techniques known in the art. For example, expression from host cells, wherein expression vector(s) encoding the DVD heavy and DVD light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the DVD proteins of the invention in either prokaryotic or eukaryotic host cells, DVD proteins are expressed in eukaryotic cells, for example, mammalian host cells, because such eukaryotic cells (and in particular mammalian cells) are more likely than prokaryotic cells to assemble and secrete a properly folded and immunologically active DVD protein.

Exemplary mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in Kaufman, R. J. and Sharp, P. A. (1982) Mol. Biol. 159:601-621), NSO myeloma cells, COS cells, SP2 and PER.C6 cells. When recombinant expression vectors encoding DVD proteins are introduced into mammalian host cells, the DVD proteins are produced by culturing the host cells for a period of time sufficient to allow for expression of the DVD proteins in the host cells or secretion of the DVD proteins into the culture medium in which the host cells are grown. DVD proteins can be recovered from the culture medium using standard protein purification methods.

In an exemplary system for recombinant expression of DVD proteins of the invention, a recombinant expression vector encoding both the DVD heavy chain and the DVD light chain is introduced into dhfr-CHO cells by calcium phosphate-mediated transfection. Within the recombinant expression vector, the DVD heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the DVD heavy and light chains and intact DVD protein is recovered from the culture medium. Standard molecular biology techniques are used to prepare the recombinant expression vector, transfect the host cells, select for transformants, culture the host cells and recover the DVD protein from the culture medium. Still further the invention provides a method of synthesizing a DVD protein of the invention by culturing a host cell of the invention in a suitable culture medium until a DVD protein of the invention is synthesized. The method can further comprise isolating the DVD protein from the culture medium.

An important feature of DVD-Ig is that it can be produced and purified in a similar way as a conventional antibody. The production of DVD-Ig results in a homogeneous, single major product with desired dual-specific activity, without any sequence modification of the constant region or chemical modifications of any kind. Other previously described methods to generate “bi-specific”, “multi-specific”, and “multi-specific multivalent” full length binding proteins do not lead to a single primary product but instead lead to the intracellular or secreted production of a mixture of assembled inactive, mono-specific, multi-specific, multivalent, full length binding proteins, and multivalent full length binding proteins with combination of different binding sites. As an example, based on the design described by Miller and Presta (PCT Publication No. WO2001/077342(A1), there are 16 possible combinations of heavy and light chains. Consequently only 6.25% of protein is likely to be in the desired active form, and not as a single major product or single primary product compared to the other 15 possible combinations. Separation of the desired, fully active forms of the protein from inactive and partially active forms of the protein using standard chromatography techniques, typically used in large scale manufacturing, is yet to be demonstrated.

The design of the “dual-specific multivalent full length binding proteins” of the present invention leads to a dual variable domain light chain and a dual variable domain heavy chain which assemble primarily to the desired “dual-specific multivalent full length binding proteins”.

At least 50%, at least 75% and at least 90% of the assembled, and expressed dual variable domain immunoglobulin molecules are the desired dual-specific tetravalent protein. This aspect of the invention particularly enhances the commercial utility of the invention. This disclosure includes a method to express a dual variable domain light chain and a dual variable domain heavy chain in a single cell leading to a single primary product of a “dual-specific tetravalent full length binding protein”.

Disclosed herein are methods of expressing a dual variable domain light chain and a dual variable domain heavy chain in a single cell leading to a “primary product” of a “dual-specific tetravalent full length binding protein,” where the “primary product” is more than 50%, 75% or 90% of all assembled protein, comprising a dual variable domain light chain and a dual variable domain heavy chain.

### Uses of DVD-Ig

Given their ability to bind to two or more antigens the binding proteins of the invention can be used to detect the antigens (e.g., in a biological sample, such as serum or plasma), using a conventional immunoassay, such as an enzyme linked immunosorbent assays (ELISA), a radioimmunoassay (RIA) or tissue immunohistochemistry. The DVD-Ig may be directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, b-galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride and phycoerythrin; an example of a luminescent material includes luminol; and examples of suitable radioactive material include 3H, 14C, 35S, 90Y, 99Tc, 111In, 1251, 1311, 177Lu, 166Ho, and 153Sm.

The binding proteins of the invention may neutralize the activity of the antigens both in vitro and in vivo. Accordingly, such DVD-Igs can be used to inhibit antigen activity, e.g., in a cell culture containing the antigens, in human subjects or in other mammalian subjects having the antigens with which a binding protein of the invention cross-reacts. A binding protein of the invention can be administered to a human subject for therapeutic purposes.

### Pharmaceutical Compositions

The invention also provides pharmaceutical compositions comprising a binding protein, of the invention and a pharmaceutically acceptable carrier. The pharmaceutical compositions comprising binding proteins of the invention are for use in, but not limited to, diagnosing, detecting, or monitoring a disorder, in preventing (e.g., inhibiting or delaying the onset of a disease, disorder or other condition), treating, managing, or ameliorating of a disorder or one or more symptoms thereof, and/or in research. The composition may further comprise a carrier, diluent or excipient.

The binding proteins of the invention can be incorporated into pharmaceutical compositions suitable for administration to a subject. Typically, the pharmaceutical composition comprises a binding protein of the invention and a pharmaceutically acceptable carrier. The term “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In some embodiments, isotonic agents, for example, sugars, polyalcohols such as mannitol, sorbitol, or sodium chloride, are included in the composition. Pharmaceutically acceptable carriers may further comprise minor amounts of auxiliary substances such as wetting or emulsifying agents, preservatives or buffers, which enhance the shelf life or effectiveness of the antibody or antibody portion.

Various delivery systems are known and can be used to administer one or more antibodies of the invention or the combination of one or more antibodies of the invention and a prophylactic agent or therapeutic agent useful for preventing, managing, treating, or ameliorating a disorder or one or more symptoms thereof, e.g., encapsulation in liposomes, microparticles, microcapsules, recombinant cells capable of expressing the antibody or antibody fragment, receptor-mediated endocytosis (see, e. g., Wu and Wu (1987) J. Biol. Chem. 262:4429-4432), construction of a nucleic acid as part of a retroviral or other vector, etc. Methods of administering a prophylactic or therapeutic agent of the invention include, but are not limited to, parenteral administration (e.g., intradermal, intramuscular, intraperitoneal, intravenous and subcutaneous), epidural administration, intratumoral administration, and mucosal administration (e.g., intranasal and oral routes). In addition, pulmonary administration can be employed, e.g., by use of an inhaler or nebulizer, and a formulation with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903. A pharmaceutical composition of the invention is formulated to be compatible with its intended route of administration.

The bispecific antibody provided herein may be expressed in AAV expression vector. AAV viral vectors have a self-processing sequence between the heavy and light chain coding sequence of the immunoglobulin allowing for expression of a functional antibody molecule from a single expression cassette driven by a single promoter. Exemplary AAV vector constructs comprise a sequence encoding a self-processing cleavage site between two Ig polypeptide chains and may further comprise an additional proteolytic cleavage site adjacent to the self-processing cleavage site for removal of amino acids derived from the self-processing site remaining following cleavage.

The practice of the present invention may employ, unless otherwise indicated, conventional techniques of cell biology, molecular biology (including recombinant techniques), microbiology, biochemistry and immunology, which are within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991), each of which is expressly incorporated by reference herein.

In view of this disclosure, the antibodies, including bispecific antibodies, disclosed herein may be prepared by means known by those skilled in the art, including those means described in U.S. Pat. Nos. 9,309,319, 9,284,357, 9,226,983, 9,212,227, 9,109,026, 9,090,694, 8,865,645, 8,785,153, 8,586,714, 8,147,817, 8,119,771, 7,714,119, 7,560,530, 7,235,641, and 6,294,353, U.S. Patent Application Publication Nos. 2015/0315290, 2015/0056251, US2014/0271642, 2014/0212412, and 2010/0260770, PCT International Application Publication Nos. WO2015106080, WO2014164959, WO2014090800, WO2012113927, and WO2005079844, Coloma 1997, Jakob 2013, Kontermann 2012, Kontermann 2011, Klein 2012, and Spiess 2015.

The subject invention provides a method of treating a patient suffering from a localized fibrotic condition which comprises administering to the patient an amount of an IL-33 antagonist effective to treat the patient.

The subject invention also provides a method of treating a patient suffering from a localized fibrotic condition which comprises administering to the patient an amount of an IL-33R antagonist or an ST2 antagonist effective to treat the patient.

If the localized fibrotic condition is Dupuytren's disease, the localized fibrotic condition is early disease stage Dupuytren's disease.

The therapeutic agents (for example, the bispecific antibody) of the present invention may be used to treat the localized fibrotic diseases when the patient is in the early stages of the localized fibrotic disease.

In another embodiment, the therapeutic agent (for example, the bispecific antibody) may be administered prophylactically, for example, to prevent recurrence of Dupuytren's diseases following surgery/needle or fasciotomy/collagenase injection. Also the therapeutic agent (for example, the bispecific antibody) may be administered prophylactically to (a) prevent periarticular fibrosis following joint injury or joint replacement, (b) prevent abdominal or pelvic adhesions following surgery, (c) prevent hypertrophic or keloid scar formation following trauma, burns, surgery, or treatment (for example surgical excision of keloid scars), (d) lead to improved appearance of cutaneous scars, for example following surgery, (e) prevent capsule formation following insertion of breast or other implants, (f) prevent adhesions and scarring around the structures following nerve or tendon surgery.

In accordance with this invention, the IL-33 antagonist may be any of the following:
a) an antibody, or antigen binding fragment of an antibody, that specifically binds to, and inhibits activation of, an IL-33 receptor;
b) a soluble form of an IL-33 receptor that specifically binds to IL-33 and inhibits IL-33 from binding to the IL-33 receptor;
c) an antisense oligonucleotide that specifically inhibits synthesis of IL-33;
d) a small molecule that specifically inhibits the activity of IL-33;
e) a bispecific antibody comprising at least one antigen binding domain of which binds to and inhibits activation of, an IL-33 receptor; or
f) an siRNA that specifically inhibits synthesis of IL-33.

In another embodiment, the IL-33 antagonist is a bispecific antibody comprising an antigen binding domain which binds to IL-33 and inhibits activation of, an IL-33 receptor; and a antigen binding domain which binds to TNF and inhibits activation of a TNF receptor.

The IL-33 antagonist may be a siRNA that specifically inhibits synthesis of ST2.

The IL-33 antagonist may be chimeric antibodies, humanized antibodies, human antibodies, and antigen binding fragments of chimeric humanized or human antibodies. In another embodiment, the IL-33 antagonist is a soluble ST2 polypeptide, a soluble IL-1RAP protein or ANB020.

The IL-33 antagonist may also be a bispecific antibody such as a
i) asymmetric IgG-like bispecific antibody;
ii) symmetric IgG-like bispecific antibody;
iii) IgG fusion bispecific antibody;
iv) Fc fusion bispecific antibody;
v) Fab fusion bispecific antibody;
vi) ScFv- or diabody-based bispecific antibody; or
vii) IgG/Non-IgG fusion bispecific antibody.

In one embodiment, the IL-33 antagonist is a RNA interference (RNAi) antagonist. In another embodiment, the IL-33 antagonist is:
a. a small interfering RNA (siRNA);
b. a short hairpin RNA (shRNA); or
c. a siRNA that specifically inhibits synthesis of IL-33.

The RNAi antagonist, the siRNA or the shRNA may be directed to and targeting the IL-33 receptor or the IL-33 receptor ST2.

The IL-33 antagonist may be administered orally, intralesionally, by intravenous therapy or by subcutaneous, intramuscular, intraarterial, intravenous, intracavitary, intracranial or intraperitoneal injection.

The IL-33 antagonist may be injected directly into the affected tissue or injected to a site of maximal cellularity or maximal inflammation.

The IL-33 antagonist or the bispecific antibody may be administered daily, weekly, monthly, bi weekly, once every two months, once every three months, once every 6 months, or once every 12 months.

The effective amount of the IL-33 antagonist may be an amount between about 0.1 mg and about 500 mg.

The method may further comprises co-administering a TNF antagonist.

The administration of the IL-33 antagonist may precede the administration of the TNF antagonist. The patient may be receiving the IL-33 antagonist prior to initiating administering the TNF antagonist and continues to receive the IL-33 antagonist after administration of the TNF antagonist is initiated.

The administration of the TNF antagonist may precede the administration of the IL-33 antagonist. The patient may be receiving the TNF antagonist prior to initiating administering the IL-33 antagonist and continues to receive the IL-33 antagonist after administration of the TNF antagonist is initiated.

In this invention, the TNF antagonist may be a RNA interference (RNAi) antagonist. The TNF antagonist may also be: (a) a small interfering RNA (siRNA); (b) a short hairpin RNA (shRNA); or (c) a siRNA that specifically inhibits synthesis of TNFR1 and/or TNFR2. The RNAi antagonist, the siRNA or the shRNA may be directed to and targeting TNFR1 and/or TNFR2. In another embodiment, the siRNA is directed to and targeting the TNF receptor 2.

The TNF antagonist may be administered in an amount between about 0.05 and about 5.0 times the clinical dose of the TNF antagonist typically administered to a patient with rheumatoid arthritis. In another embodiment, the amount of the TNF antagonist is between about 5 mg and about 300 mg.

The TNF antagonist may be one or more of infliximab, adalimumab, certolizumab pegol, golimumab or etanercept or their biosimilars.

In one embodiment, the TNF antagonist is golimumab and the amount of golimumab administered is between about 1 mg and about 90 mg.

In one embodiment, the TNF antagonist is adalimumab and the amount of adalimumab administered is between about 5 mg and about 100 mg.

In one embodiment, the TNF antagonist is certolizumab pegol and the amount of certolizumab pegol administered is between about 50 mg and about 200 mg.

In one embodiment, the TNF antagonist is infliximab and the amount of infliximab administered is between about 50 mg and about 300 mg.

In one embodiment, the TNF antagonist is etanercept and the amount of etanercept administered is between about 5 mg and about 50 mg.

The TNF antagonist may be a TNF receptor 2 (TNFR2) antagonist, an antisense oligonucleotide, a RNA interference (RNAi) antagonist, a siRNA, or a shRNA.

The method may further comprise co-administering a GM-CSF antagonist.

The administration of the IL-33 antagonist may precede the administration of the GM-CSF antagonist. Additionally, the patient may receive the IL-33 antagonist prior to initiating administering the GM-CSF antagonist and continue to receive the IL-33 antagonist after administration of the GM-CSF antagonist is initiated.

The administration of the GM-CSF antagonist may precede the administration of the IL-33 antagonist. The patient may receive the GM-CSF antagonist prior to initiating administering the IL-33 antagonist and continue to receive the IL-33 antagonist after administration of the GM-CSF antagonist is initiated.

The method may further comprise co-administering one or more of an IL-17 antagonist, an IL-21 antagonist or an IL-23 antagonist.

The administration of the IL-33 antagonist may precede the administration of the one or more of the IL-17 antagonist, the IL-21 antagonist, or the IL-23 antagonist. Additionally, the patient may receive the IL-33 antagonist prior to initiating administering the one or more of the IL-17 antagonist, the IL-21 antagonist, or the IL-23 antagonist and continue to receive the IL-33 antagonist after administration of the one or more of the IL-17 antagonist, the IL-21 antagonist, or the IL-23 antagonist is initiated.

The administration of the one or more of the IL-17 antagonist, the IL-21 antagonist, or the IL-23 antagonist may precede the administration of the IL-33 antagonist. The patient may be receiving the one or more of the IL-17 antagonist, the IL-21 antagonist, or the IL-23 antagonist prior to initiating administering the IL-33 antagonist and continue to receive the IL-33 antagonist after administration of the one or more of the IL-17 antagonist, the IL-21 antagonist, or the IL-23 antagonist is initiated.

In one embodiment, the amount of the one or more of the IL-17 antagonist, the IL-21 antagonist, or the IL-23 antagonist is between about 10 mg and about 300 mg.

The method may further comprises administering of a therapeutically, prophylactically or progression-inhibiting amount of a DAMP antagonist and/or an AGE inhibitor to the patient.

A DAMP antagonist may be administered and the DAMP antagonist is an Alarmin antagonist.

In one embodiment, the Alarmin antagonist is one or more of an antagonist of HMGB1, an antagonist of S100A8, an antagonist of S100A9, an antagonist of SI00A8/9, and a heat shock protein.

The methods of treatment of the present invention may result in alleviation of a symptom of Dupuytren's disease, frozen shoulder (adhesive capsulitis), periarticular fibrosis, keloid or hypertrophic scars, capsules around implants or prostheses, endometriosis, abdominal adhesions, perineural fibrosis, Ledderhose disease, Peyronie's disease, peritendinous adhesions, or periarticular fibrosis. The method of treatment may also result in improvement of the patient's quality of life or general health status.

A IL-33 receptor antagonist may be used instead of an IL-33 antagonist.

The antagonists of the present invention may be administered by injection together using a twin barreled syringe or at intervals separated by minutes to days. The antagonists may also be administered in a single syringe needle with the use of bispecific antibodies.

The methods of the present invention may further comprise co-administering one or more or human matrix metalloproteinases or collagenase Clostridium histolyticum (Xiaflex®). The human matrix metalloproteinase can be the native enzyme or modified to restrict activity, for example calcium dependent.

The subject invention also provides a method of treating a patient suffering from a localized fibrotic condition which comprises administering to the patient an amount of a TNF receptor 2 (TNFR2) antagonist effective to treat the patient.

If the localized fibrotic condition is Dupuytren's disease, it may be early disease state Dupuytren's disease.

The TNFR2 antagonist may be
a) an antibody, or antigen binding fragment of an antibody, that specifically binds to, and inhibits activation of, an TNFR2;
b) a soluble form of an TNFR2 that specifically binds to TNFR2 and inhibits TNFR2 from binding to the TNFR2;
c) an antisense nucleic acid that specifically inhibits synthesis of TNFR2;
d) a siRNA that specifically inhibits synthesis of TNFR2;
e) a small molecule that specifically inhibits the activity of TNFR2; or
f) a bispecific antibody comprising at least one antigen binding domain of which binds to and inhibits activation of, an TNFR2; or
g) an antisense oligonucleotide.

The TNFR2 antagonist may be an antibody selected from the group consisting of chimeric antibodies, humanized antibodies, human antibodies, and antigen binding fragments of chimeric humanized and human antibodies.

The TNFR2 antagonist may be a bispecific antibody which is a
i) asymmetric IgG-like bispecific antibody;
ii) symmetric IgG-like bispecific antibody;
iii) IgG fusion bispecific antibody;
iv) Fc fusion bispecific antibody;
v) Fab fusion bispecific antibody;
vi) ScFv- or diabody-based bispecific antibody; and
vii) IgG/Non-IgG fusion bispecific antibody.

In one embodiment, the TNFR2 antagonist is a RNA interference (RNAi) antagonist.

The TNFR2 antagonist may also be:
a. a small interfering RNA (siRNA);
b. a short hairpin RNA (shRNA); or
c. a siRNA that specifically inhibits synthesis of TNFR2.

The TNFR2 antagonist may be administered orally, intralesionally, by intravenous therapy or by subcutaneous, intramuscular, intraarterial, intravenous, intracavitary, intracranial, or intraperitoneal injection.

In one embodiment, the TNFR2 antagonist is injected directly into the affected tissue. In another embodiment, the TNFR2 antagonist is injected to a site of maximal cellularity or maximal inflammation.

In one embodiment, the TNFR2 antagonist is administered daily. In another embodiment, the TNFR2 antagonist is administered weekly. In a further embodiment, the TNFR2 antagonist is administered monthly. The TNFR2 antagonist may be administered once every three months, once every 6 months, or once every 12 months.

The TNFR2 antagonist may be administered in an amount between about 5 mg and about 300 mg.

The method may further comprise administering a therapeutically, prophylactically or progression-inhibiting amount of a DAMP antagonist and/or an AGE inhibitor to the patient. In another instance of this invention, a DAMP antagonist is administered and the DAMP antagonist is an Alarmin antagonist.

The Alarmin antagonist may be one or more of an antagonist of HMGB1, an antagonist of S100A8, an antagonist of S100A9, an antagonist of SI00A8/9, and a heat shock protein.

The invention also provides a method of treating a patient suffering from a localized fibrotic condition which comprises administering to the patient an amount of a bispecific antibody comprising
a) a IL-33 antigen binding domain of which (i) binds to and inhibits activation of, an IL-33 receptor, or (ii) specifically binds to IL-33 and inhibits IL-33 from binding to the IL-33 receptor, and
b) a TNF-α antigen binding domain of which (i) binds to and inhibits activation of, a TNF-α receptor, or (ii) specifically binds to TNF-α and inhibits TNF-α from binding to the TNF-α receptor,
   wherein the bispecific antibody is effective to treat the patient.

The IL-33 antigen binding domain of which binds to and inhibits activation of an IL-33 receptor may be referred to as the IL-33 antibody domain of the bispecific antibody. Likewise, the IL-33 antigen binding domain of which specifically binds to IL-33 and inhibits IL-33 from binding to the IL-33 receptor may be referred to as the IL-33 soluble receptor domain of the bispecific antibody.

The TNF antigen binding domain of which binds to and inhibits activation of, a TNF receptor may be referred to as the TNF antibody domain of the bispecific antibody. Similarly, the TNF antigen binding domain of which specifically binds to TNF and inhibits TNF from binding to the TNF receptor may be referred to as the TNF soluble receptor domain of the bispecific antibody.

The bispecific antibody may comprise: (a) a IL-33 antibody domain and a TNF antibody domain, (b) a IL-33 antibody domain and a TNF soluble receptor domain, or (c) a IL-33 soluble receptor domain and a TNF soluble receptor domain.

If the patient is suffering from periarticular fibrosis, in one instance, the patient began suffering from periarticular fibrosis after an injury or after an arthroplasty.

If the localized fibrotic condition is scars, the localized fibrotic condition is preferably keloid scars, hypertrophic scars, burn scars, post traumatic or post-surgical scars.

In accordance with this invention, the bispecific antibody may be any of the following
i) asymmetric IgG-like bispecific antibody,
ii) symmetric IgG-like bispecific antibody,
iii) IgG fusion bispecific antibody,
iv) Fc fusion bispecific antibody,
v) Fab fusion bispecific antibody,
vi) ScFv- or diabody-based bispecific antibody,
vii) IgG/Non-IgG fusion bispecific antibody, or
viii) fragment-based bispecific antibody.

The bispecific antibody may be a bispecific monoclonal antibody inhibitor or a viral vector. The bispecific antibody may also be expressed in an adeno-associated virus (AAV) expression vector. The bispecific antibody may be a combined single-chain variable fragment (scFv) construct. The bispecific antibody may be made by a dual variable antibody approach. The bispecific antibody may further comprises a transcriptional promotor that is expressed only in myofibroblasts.

The TNF antagonist and/or the IL-33 antagonist may be an aptamer which functions as an antagonist.

Aptamers for use in the invention may also be aptamer constructs comprising an aptamer that binds TNF and an aptamer that binds IL-33. For example, the invention may comprises a method of treating a patient suffering from a localized fibrotic condition which comprises coadministering a patient an amount of an aptamer construct comprising an aptamer that binds TNF and an aptamer that binds IL-33 so as to treat the patient.

In some embodiments, a therapeutic effect may be achieved by administering a IL-33 aptamer, a TNF aptamer, and/or a IL-33/TNF aptamer construct such that the aptamer or aptamer construct is exposed to, and can bind to, IL-33 and/or TNF. In some embodiments, such binding occurs regardless of the method of delivery of the aptamer to the subject being treated. In some embodiments, the therapeutic effect may be achieved by administering the IL-33 aptamer, TNF aptamer, or IL-33/TNF aptamer construct such that it is exposed to, and binds to, IL-33 and/or TNF and prevents or reduces the binding of IL-33 and/or TNF to one or more cell receptors.

In some embodiments, a IL-33 aptamer, a TNF aptamer, or a IL-33/TNF aptamer construct is administered with one or more additional active agents. Such administration may be sequential or in combination.

The IL-33 antigen binding domain may be a direct IL-33 antagonist or an anti-IL-33 receptor antagonist. The IL-33 antigen binding domain may also be the binding domains of an antibody which is a chimeric antibody, humanized antibody, human antibody, or an antigen binding fragment of a chimeric humanized and human antibody.

The IL-33 antigen binding domain may be the binding domains of a soluble ST2 polypeptide, a soluble IL-1RAP protein or ANB020. The IL-33 antigen binding domain may specifically target the IL-33 receptor.

In the present invention, the IL-33 antigen binding domain may (a) bind to the cytokine IL-33, preferably neutralizing biological function, (b) be an antibody to the cytokine IL-33, (c) be an antibody to IR-1R4, (d) be an antibody to IR-1R3 (e) is a IL-1R4 soluble receptor, or (f) be a IL-1R3 soluble receptor.

The TNF antigen binding domain may binds to and inhibit TNF from binding to TNFR1 or may bind to and inhibit TNF from binding to TNFR2.

In the present invention, the TNF receptor may be TNF receptor 1 or TNF receptor 2.

In accordance with this invention, the effective amount of the bispecific antibody may be an amount between about 0.1 mg and about 500 mg or between about 0.1 mg and about 100 mg.

The bispecific antibody may be administered in an amount such that the amount of the TNF antigen binding domain is between about 0.05 and about 5.0 times the clinical dose of the TNF antigen binding domain typically administered to a patient with rheumatoid arthritis. The TNF antigen binding domain may be an infliximab construct, adalimumab construct, certolizumab pegol construct, golimumab construct or etanercept construct.

In accordance with this invention, the TNF receptor may be: (a) a TNF receptor 1 (TNFR1) and a TNF receptor 2 (TNFR2), (b) a TNFR1, or (c) a TNFR2. Additionally, the TNF antigen binding domain may inhibit both TNFR1 and TNFR2.

For the foregoing embodiments, each embodiment disclosed herein is contemplated as being applicable to each of the other disclosed embodiments. In addition, the elements recited in the packaging and pharmaceutical composition embodiments can be used in the method and use embodiments described herein.

### Pharmaceutically Acceptable Salts

The active compounds for use according to the invention may be provided in any form suitable for the intended administration. Suitable forms of the pre- or prodrug or functionally active protein produced as an active pharmaceutical ingredient, through recombinant DNA technology, include pharmaceutically (i.e. physiologically) acceptable salts, formulations, and excipients, known to those skilled in the art, for the compound(s) of the invention.

The antagonists of the present invention may act by RNA interference. Additionally, the antagonists of the present invention include, but are not limited to, siRNAs or shRNAs.

RNA interference (RNAi) refers to a process in which RNA molecules modulate and/or silence gene expression (Lagana 2015). Small interfering RNAs (siRNA) are a class of double-stranded RNA molecules which have a variety of known effects, including interference with the expression of specific genes expression (Lagana 2015). Short hairpin RNA (shRNA) refers to sequences of RNA that make tight hairpin turns that can be used to silence gene expression (Paddison 2002).

In one embodiment, the RNAi antagonist is an siRNA antagonist.

In one embodiment, said siRNA antagonist is an siRNA formed after transcription from a plasmid (RNAi expression vector) or exogenous synthesis.

In one embodiment, said siRNA is a short hairpin siRNA formed after transcription from a single promoter of said plasmid (RNAi expression vector).

In one embodiment, said siRNA is a short dsRNA formed after transcription from two flanking convergent promoters on said plasmid (RNAi expression vector).

In one embodiment, said siRNA is around 19-30 nucleotides in length.

In one embodiment, said siRNA is 21-23 nucleotides in length.

In one embodiment, said siRNA is a fragment generated by nuclease dicing of longer double-stranded RNAs at least 25, 50, 100, 200, 300, 400, or 400-800 bases in length.

In one embodiment, said siRNA is double stranded, and includes short overhangs at one or both ends.

In one embodiment, said short overhang is 1-6 nucleotides in length at the 3′ end, 2 to 4 nucleotides in length at the 3′ end, or 1-3 nucleotides in length at the 3′ end.

In one embodiment, one strand of said siRNA has a 3′ overhang, and the other strand is blunt-ended, or also has an overhang of the same or different length.

In one embodiment, said 3′ overhang is stabilized against degradation.

In one embodiment, said 3′ overhang is stabilized against degradation by including purine nucleotides adenosine or guanosine.

In one embodiment, said 3′ overhang is stabilized against degradation by substituting pyrimidine nucleotides by modified analogues, e.g., substitution of uridine nucleotide 3′ overhangs by 2′-deoxythymidine.

In one embodiment, said siRNA is chemically synthesized.

In one embodiment, said RNAi comprise either long stretches of double stranded RNA identical or substantially identical to said target nucleic acid sequence, or short stretches of double stranded RNA identical to substantially identical to only a region of said target nucleic acid sequence.

In mammals, both genome-wide and subgenomic, focused libraries of synthetic siRNAs and shRNA expression constructs are widely used (Silva 2008; Luo 2009; Barbie 2009).

The RNAi process and the use of siRNAs and shRNAs are known in the art and may be found in the following publications which are hereby incorporated by reference: U.S. Patent Publication Nos. 20130330730A1 and 20060003915; U.S. Pat. Nos. 7,893,243, 8,735,064, 6,506,559, 8,420,391, 7,560,438, and 7,416,849; PCT International Publication Nos. WO 2004076629 WO 1999/32619, WO 2001/68836, WO 2001/77350, WO 2000/44895, WO 2002/055692 and WO 2002/055693; Rao 2010, and Kanasty 2013.

The RNA molecules of the present invention that act by RNAi may be administered directly or be expressed in vivo from a suitable construct. Additionally, the siRNA and shRNA of the present invention may also be administered directly or be expressed in vivo from a suitable construct.

The RNA molecules of the present invention involved in RNAi includes chemically modified RNA molecules. Likewise, the siRNAs of the present invention includes chemically modified siRNAs. Additionally, the shRNAs of the present invention includes chemically modified shRNAs. Examples of chemically modified RNA molecules, chemically modified siRNAs and chemically modified shRNAs include, but are not limited to, those listed in the following publications: U.S. Pat. Nos. 7,956,176, 8,541,385, 8,871,730, 8,618,277, and 9,181,551; Dar 2015, Gaglione 2010, Deleavey 2012, and Chiu 2003.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

By systematically unraveling the signaling pathways, TNF was identified as a novel therapeutic target to down regulate myofibroblasts, the cells responsible for matrix deposition and contraction in Dupuytren's disease (DD) (Verjee, 2013). Anti-TNF drugs have been used for more than 10 years to treat inflammatory conditions including rheumatoid arthritis, psoriatic arthritis, juvenile arthritis, Crohn's colitis, ankylosing spondylitis and psoriasis. Although these drugs can reduce the disease-associated inflammation, they do not reverse the underlying mechanisms that drive inflammation. As a result, they have to be administered at regular intervals. Whilst TNF inhibition could be used clinically to treat early Dupuytren's disease or to prevent recurrence, it will also likely need to be injected repeatedly on a regular basis, as for rheumatoid arthritis (Taylor, 2009). A survey showed a high acceptance rate for one injection per year but this fell sharply when frequency of injection was increased to 3 per year (Table 1).

TABLE 1
Summary of responses to questionnaire regarding acceptability
of injection therapy that would prevent the progression of
disease and hence avoid the necessity of future surgery.

| | | Patients post | |
|---|---|---|---|
| Extremely or | Patients with early | surgery for | |
| very likely | Dupuytren's disease | Dupuytren's disease | Combined |
| accept: | (n = 14) | (n = 17) | (n = 31) |
| 1 injection/ | 93% | 94% | 94% |
| yr for lifetime | | | |
| 3 injections/ | 57% | 71% | 65% |
| yr for lifetime | | | |

Targeting the pathway that drives chronicity will likely reduce the frequency of anti-TNF injections necessary to control progression of the disease. IL-33 is likely one of the important factors responsible for the chronic inflammation seen in Dupuytren's disease and related disorders such as frozen shoulder and Peyronie's disease.

IL-33 is the most recently described member of the IL-1 family of cytokines and plays an important role in fibrotic disorders in a variety of tissues (Palmer, 2011). Its expression is limited to fibroblasts, myofibroblasts, smooth muscle, epithelial and dendritic cells (Schmitz, 2005) and is markedly increased by pro-inflammatory cytokines (Xu, 2008). It has been shown to play a key role in fibrotic disorders in a variety of tissues, including the skin (Rankin, 2010) and gut (Sponheim, 2010). In active lesions of ulcerative colitis, myofibroblasts are the major source of IL-33 (Kobori, 2010). IL-33 can activate inflammatory cells, including mast cells and macrophages via the IL-33 receptor to secrete pro-inflammatory cytokines, in particular TNF, and systemic anti-TNF therapy can reduce circulating IL-33 levels (Pastorelli, 2010). Fibroblasts also secrete IL-33 in response to mechanical strain in vitro (Kunisch, 2012). This is particularly pertinent as strain is crucial to the development and persistence of myofibroblasts; on loss of tension, they disassemble their α-SMA within hours (Hinz, 2001). However, the precise role of IL-33 in driving musculoskeletal and other localized fibrotic diseases such as endometriosis, abdominal adhesions, adhesive capsulitis, hypertrophic scars or keloid scars, Ledderhose disease and Peyronie's disease is not clear.

Whilst best known as effector cells in allergic responses, mast cells are now recognised as important physiological regulators of the innate and adaptive immune response, smooth muscle contraction and wound healing (Bischoff, 2007). Mast cells constitutively express the IL-33 receptor, and on exposure to IL-33, secrete pro-inflammatory cytokines including TNF without degranulation (Moulin, 2007). Whilst the differentiation and function of myofibroblasts can be regulated by mast cells (Gailit, 2001), the precise contribution of mast cell derived pro-inflammatory cytokines in driving myofibroblast formation in Dupuytren's disease and other localized fibrotic disorders has not been established.

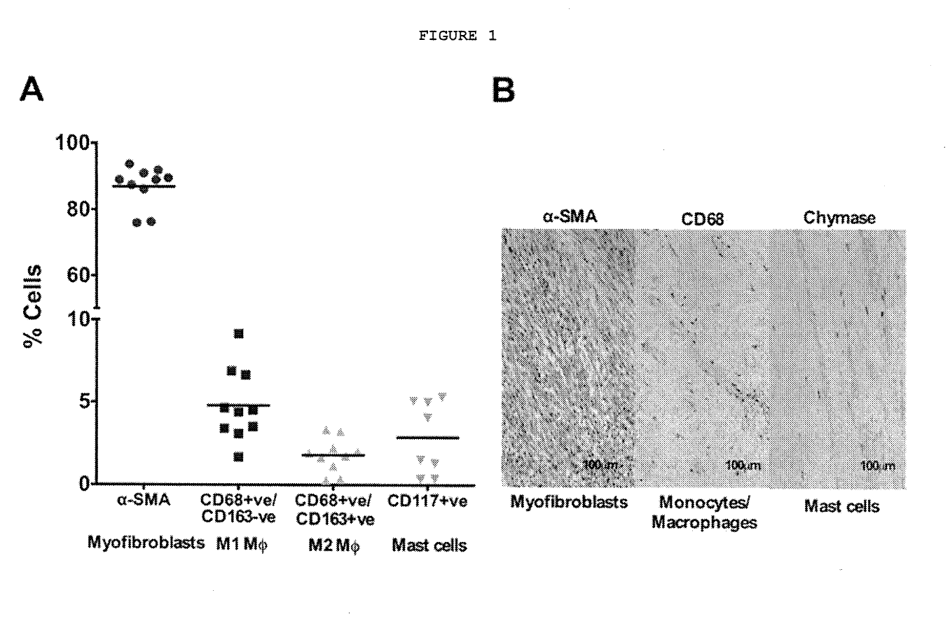

Dupuytren's tissue has been shown to be composed mainly of myofibroblasts and about 7% of all cells comprise macrophages, predominantly of the M1 phenotype. Significant numbers of mast cells have been found in Dupuytren's disease tissue (FIG. 1).

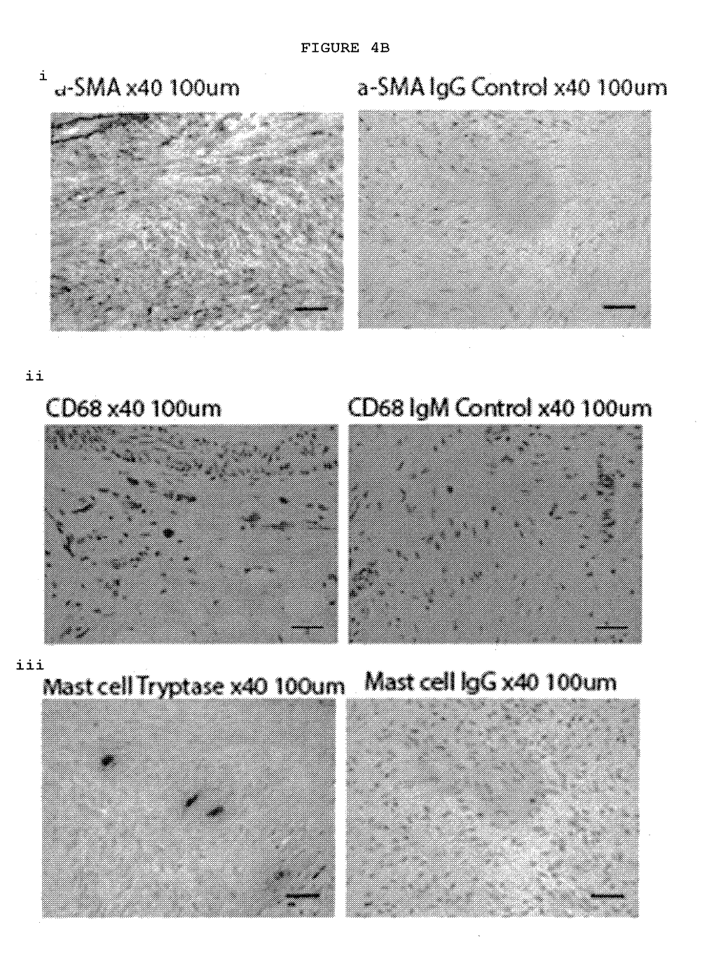

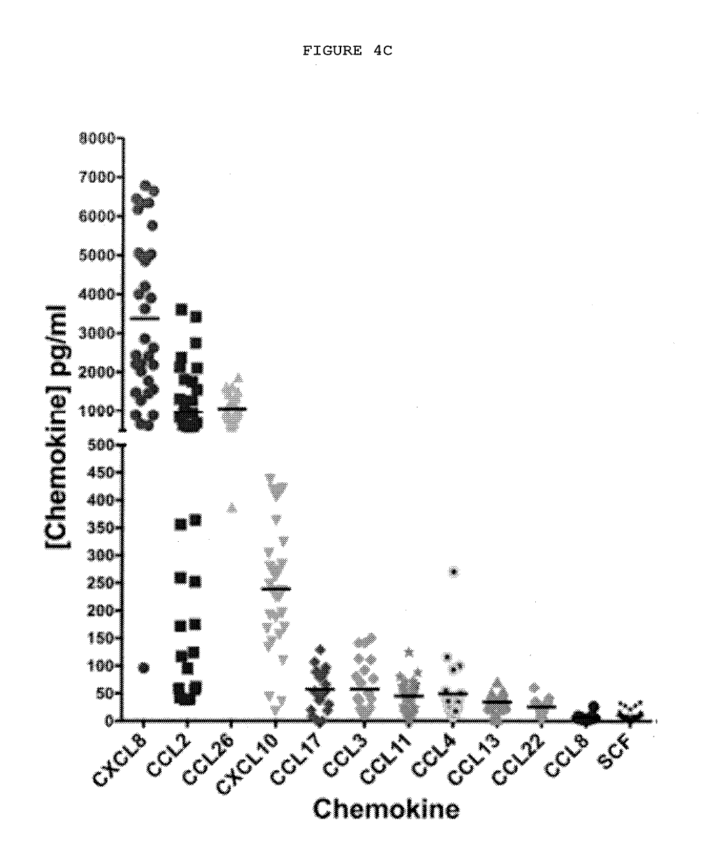

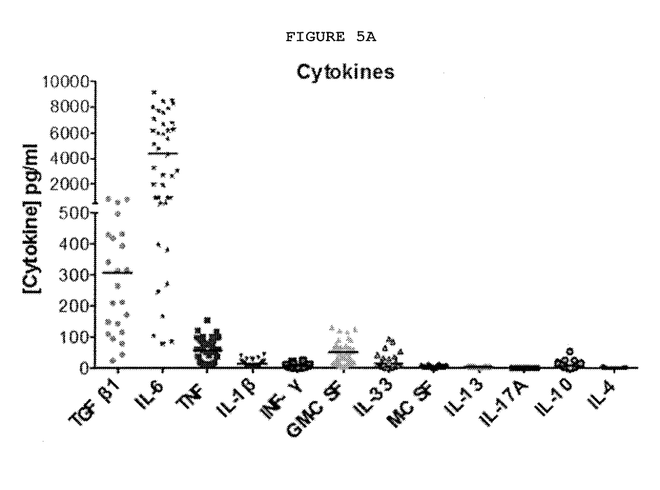

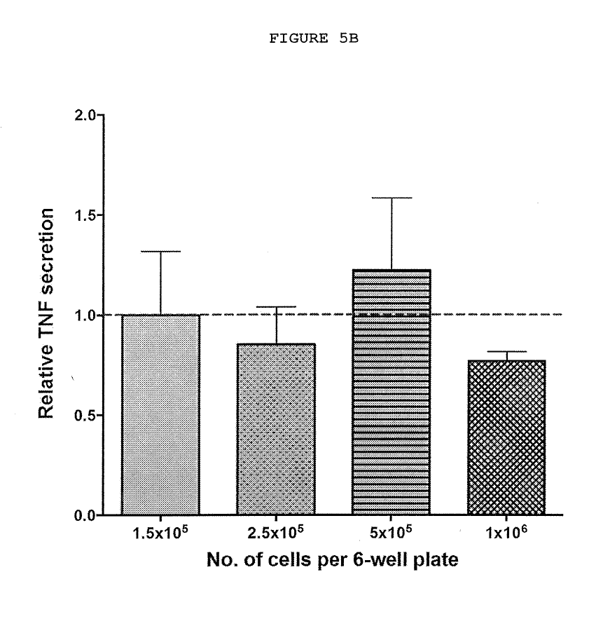

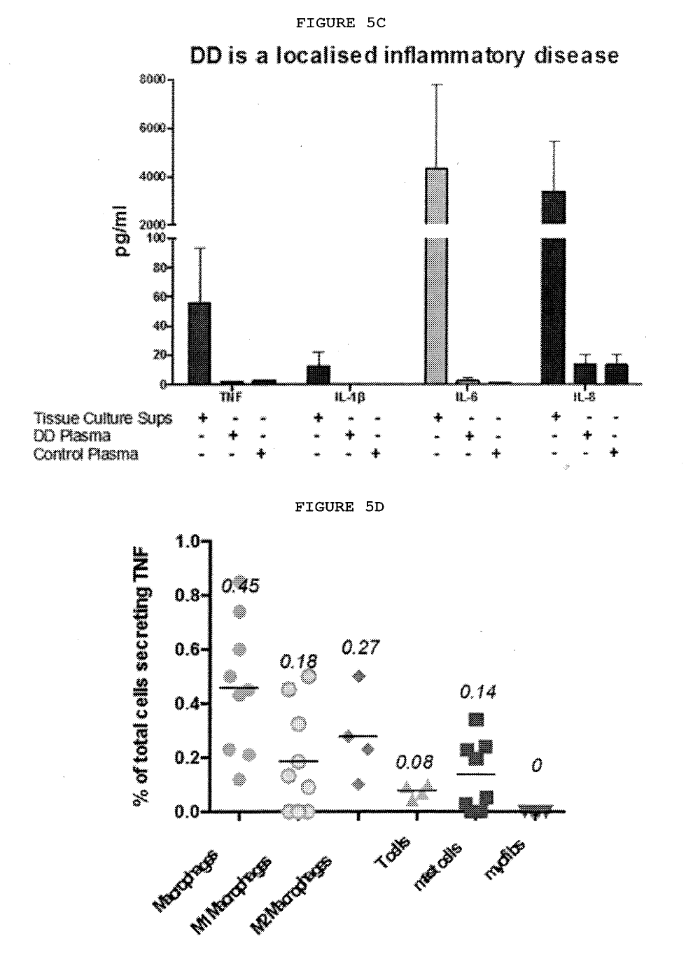

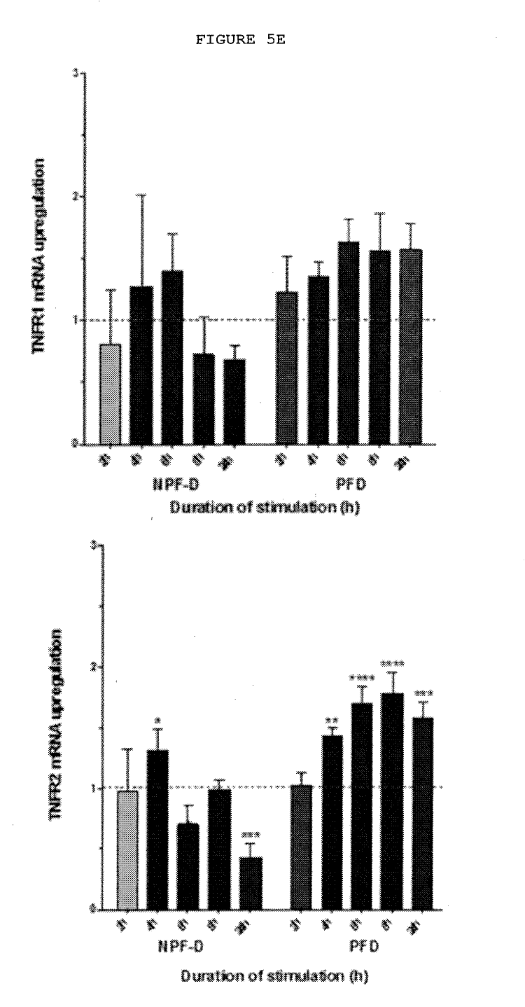

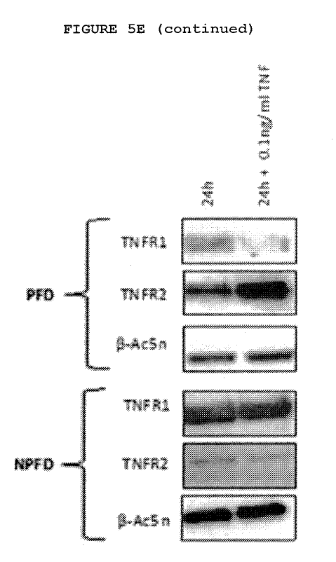

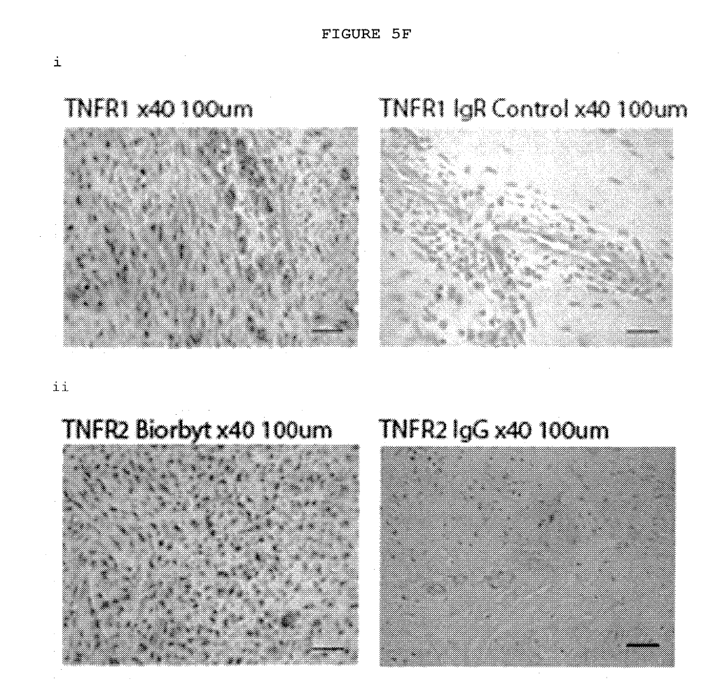

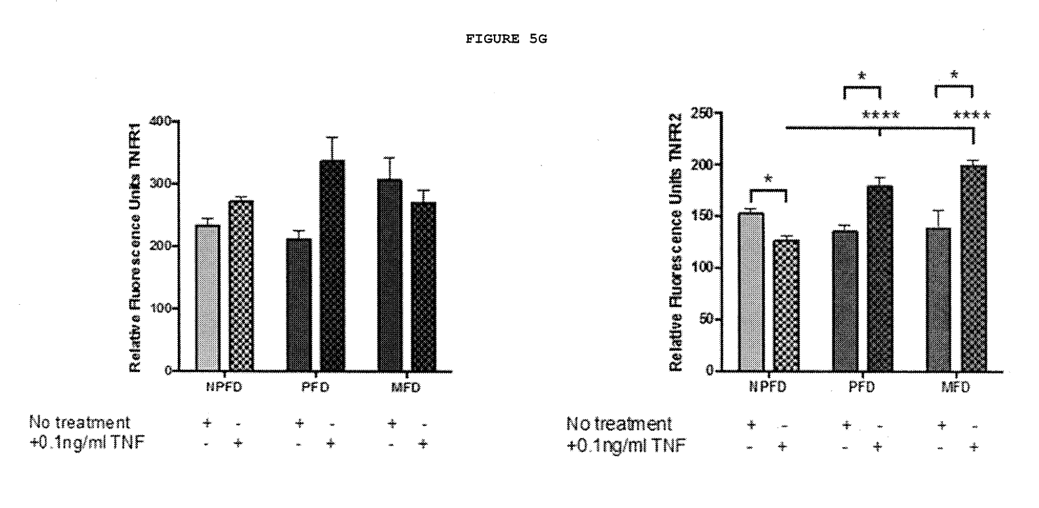

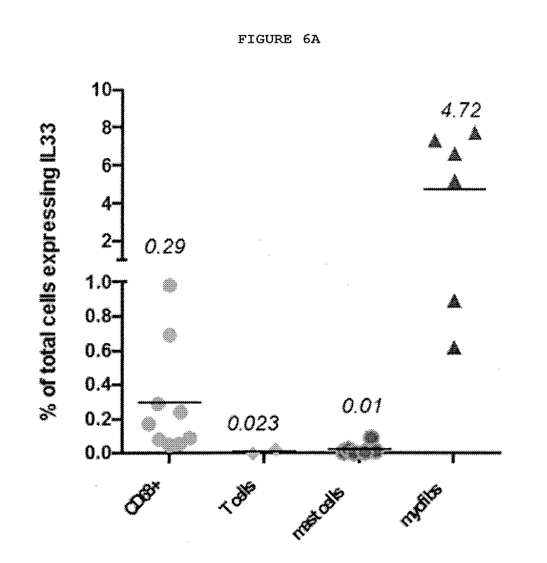

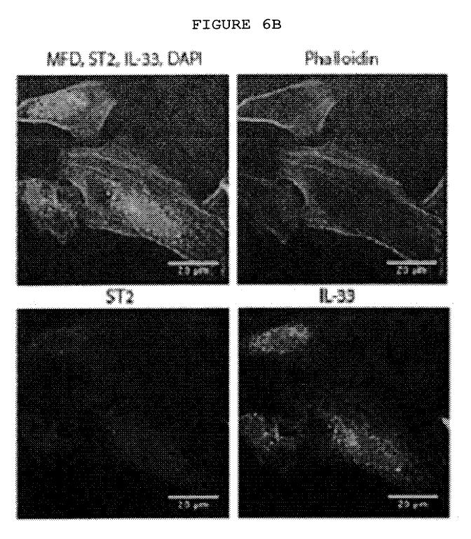

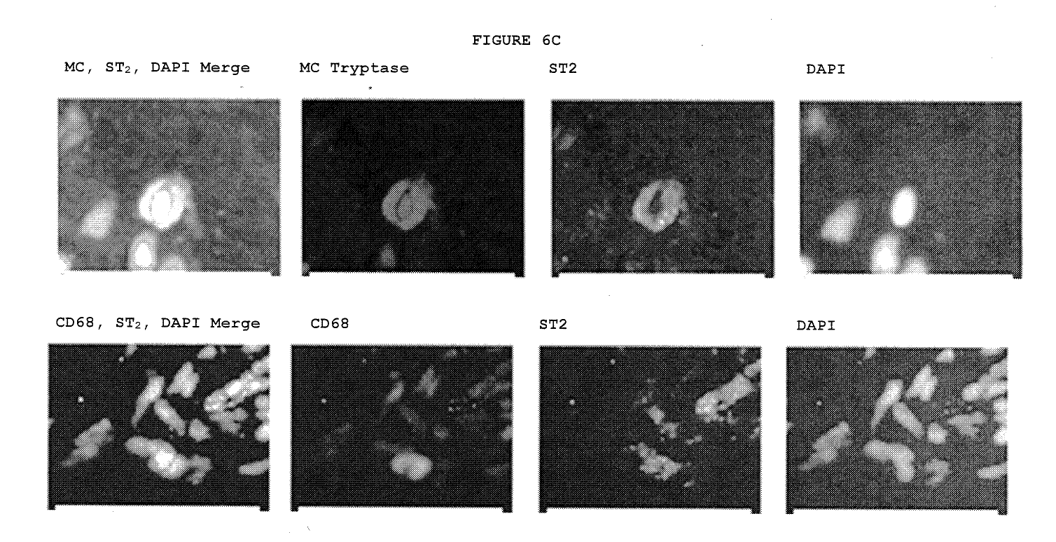

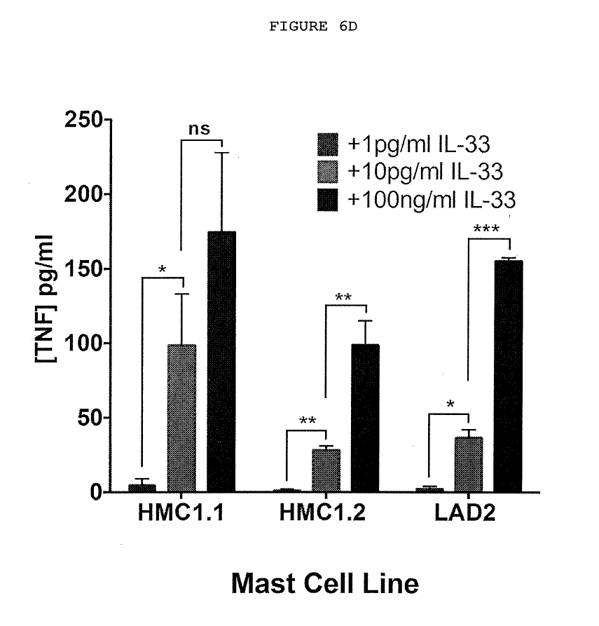

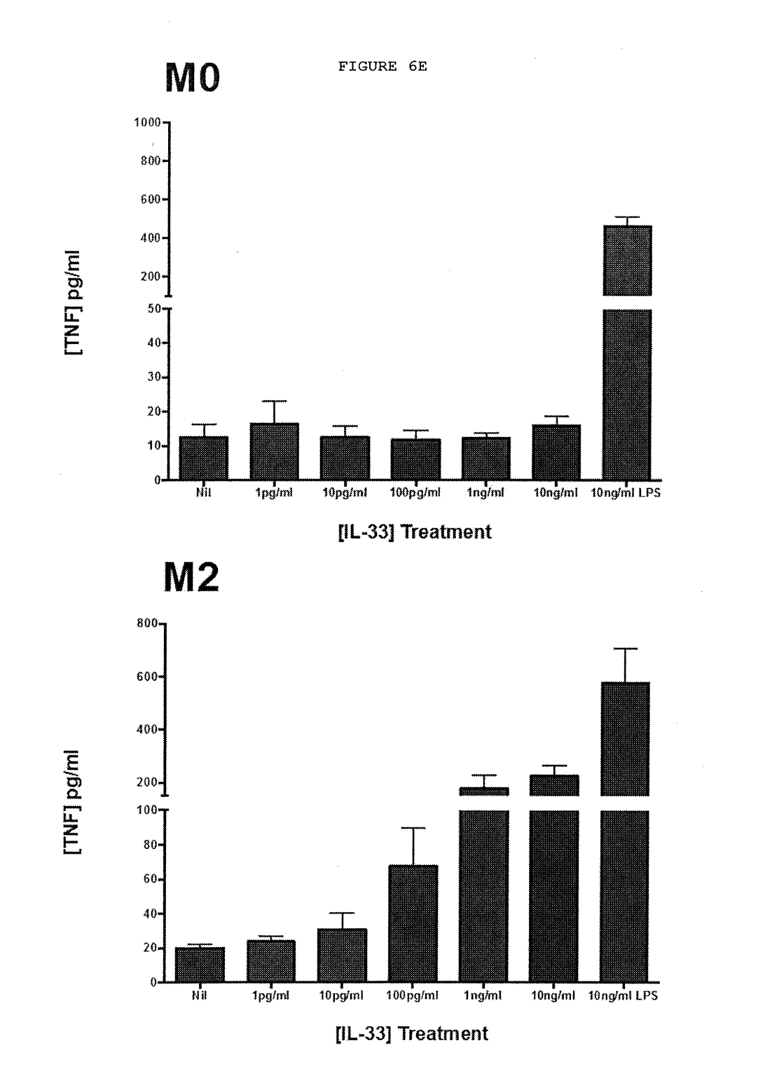

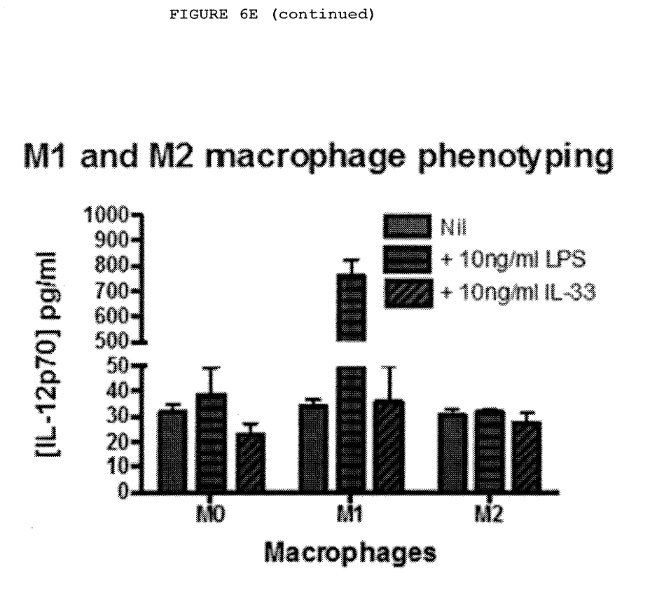

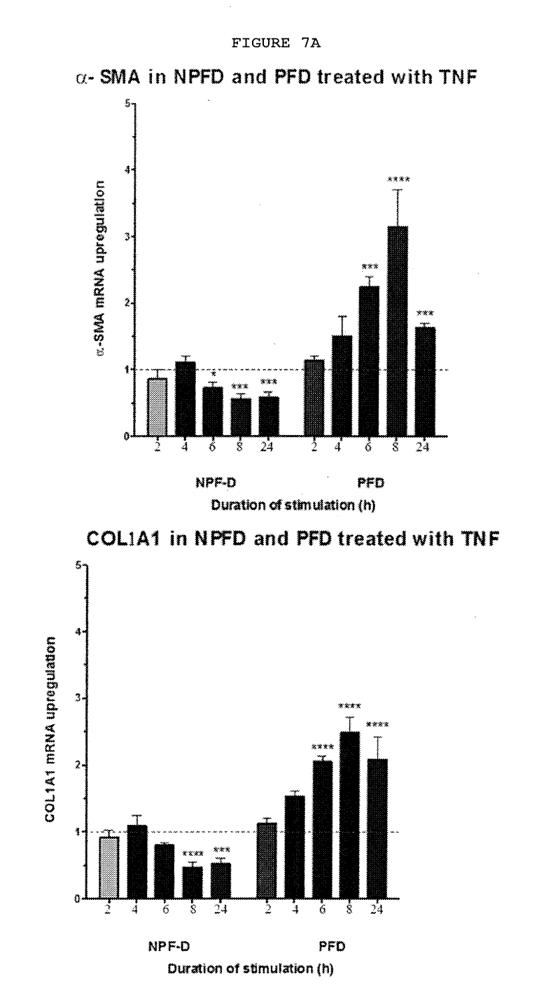

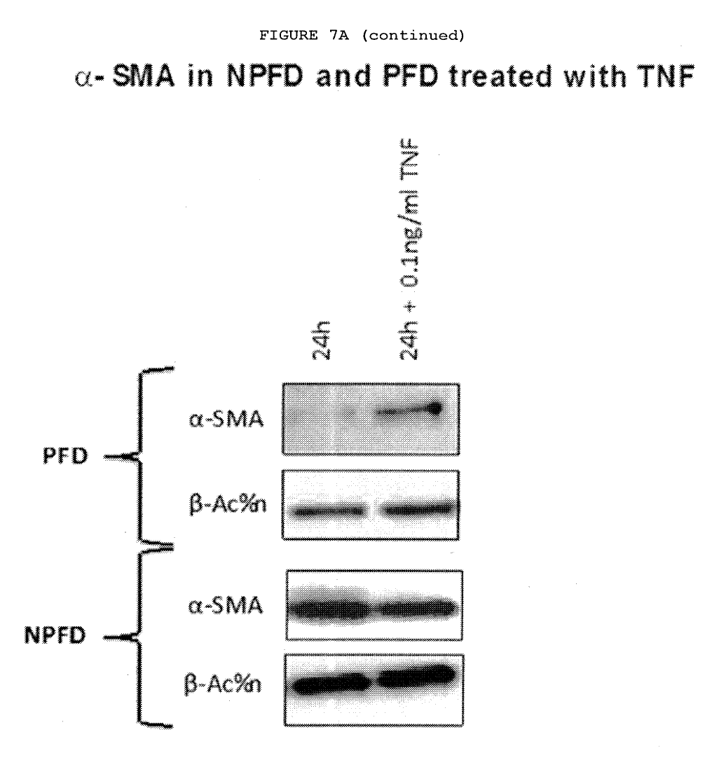

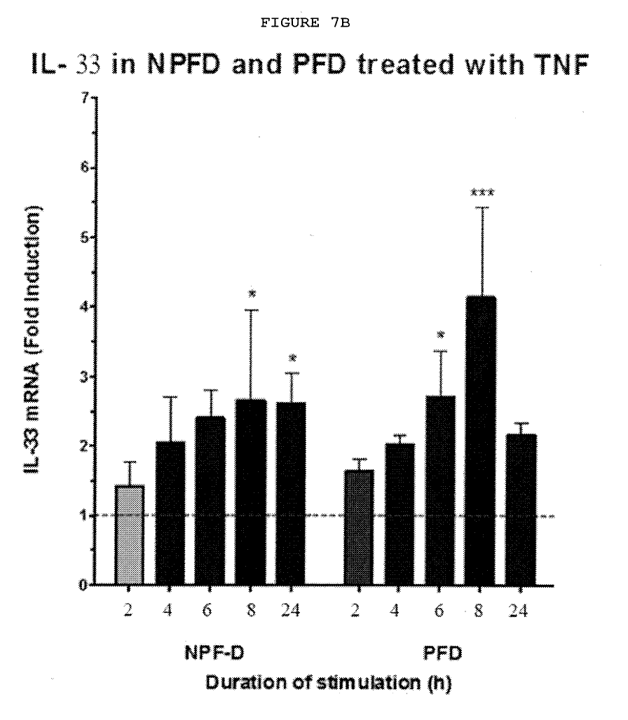

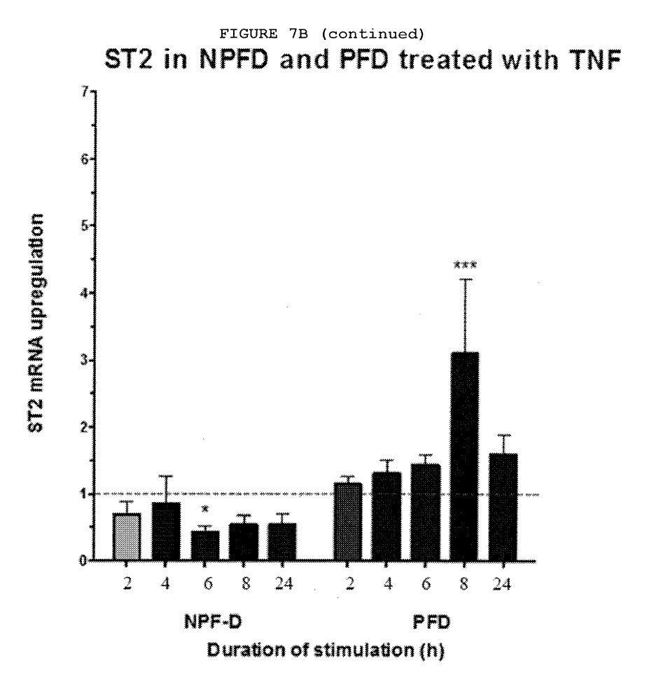

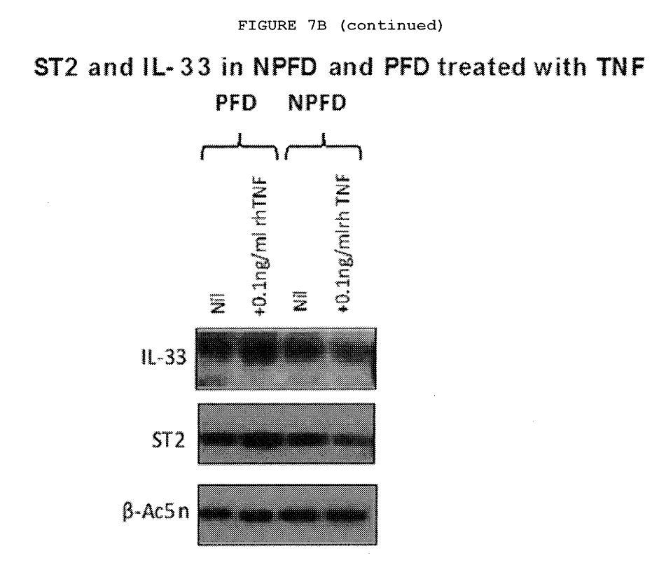

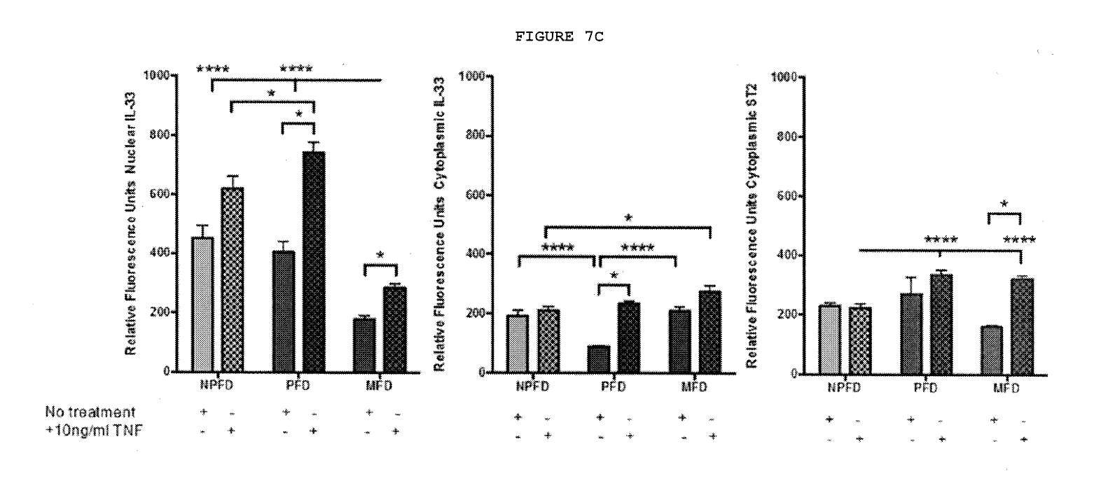

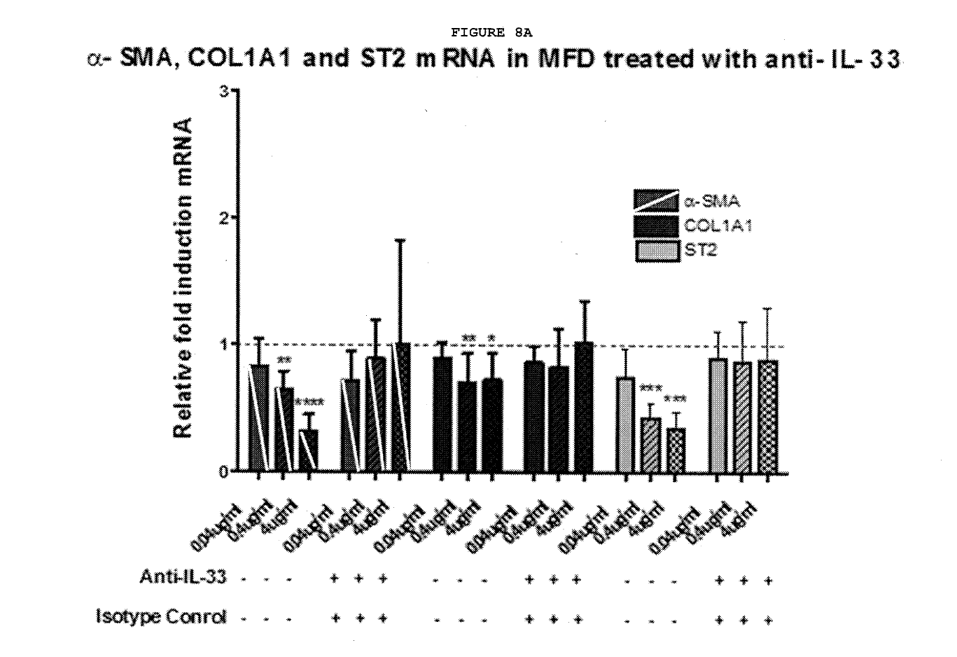

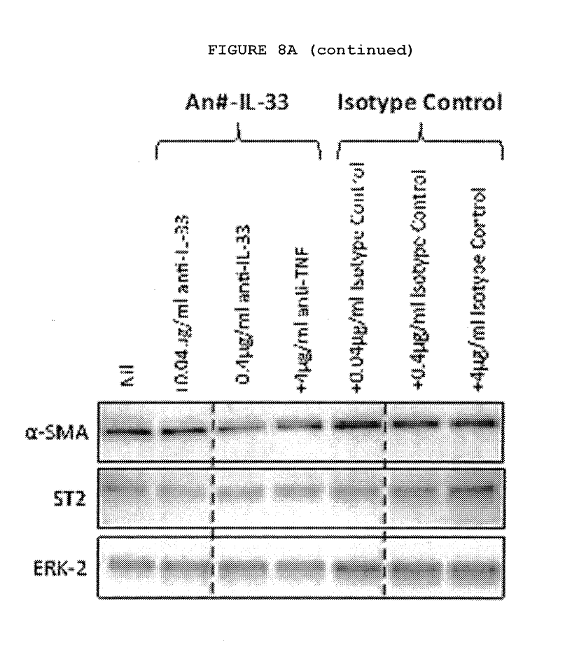

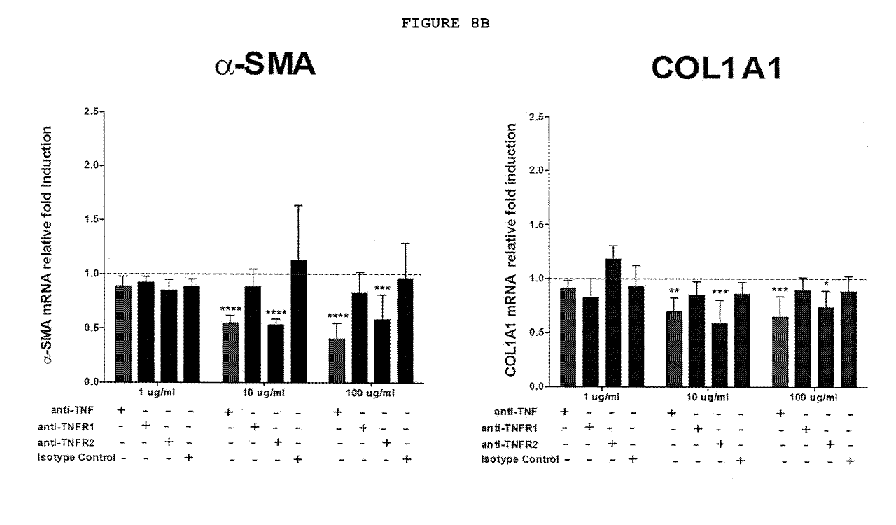

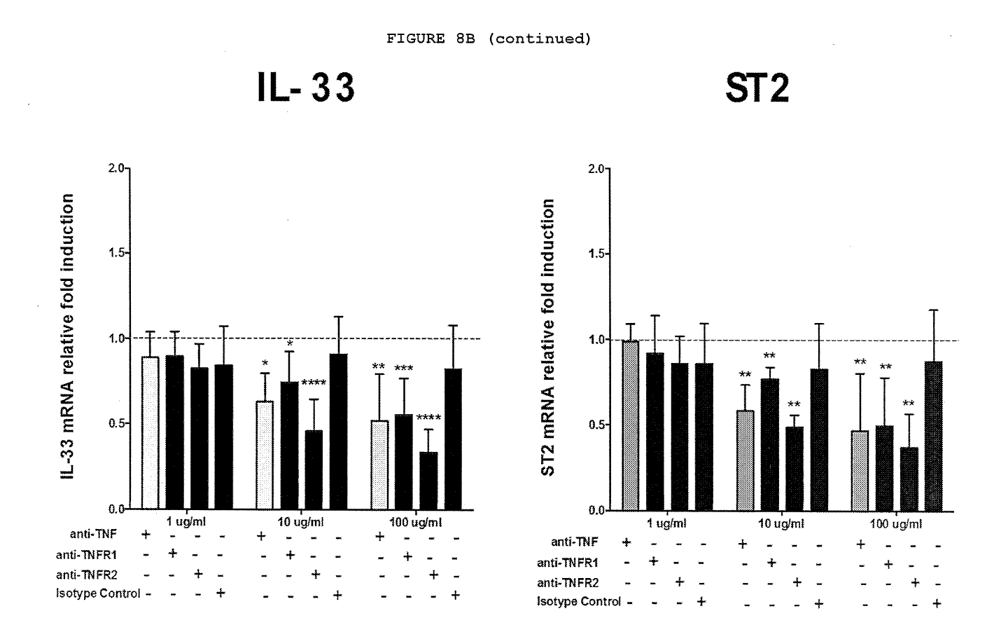

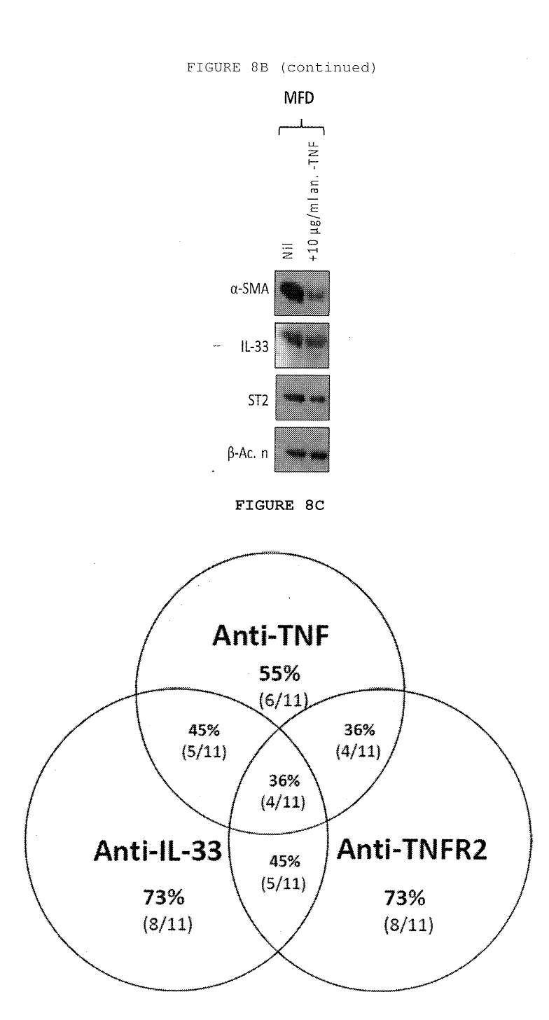

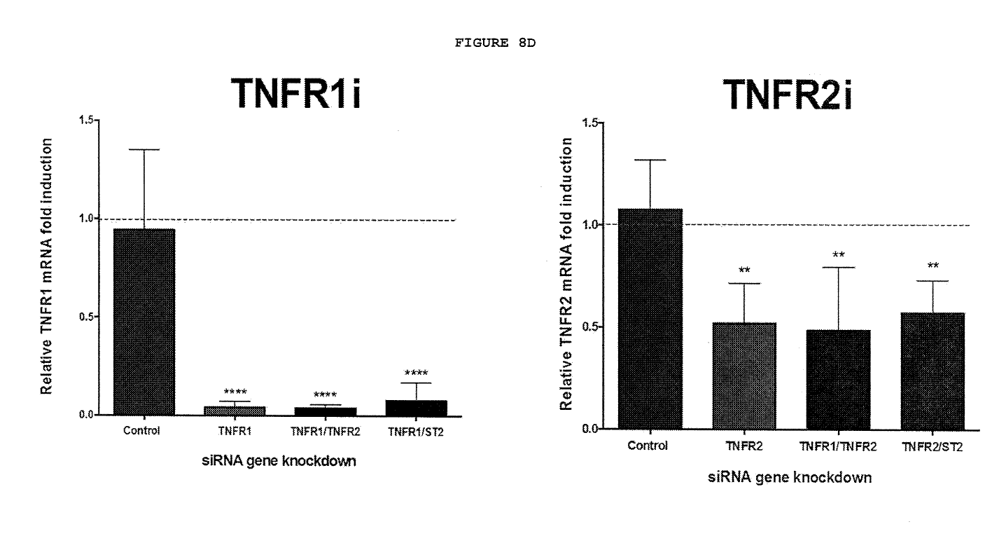

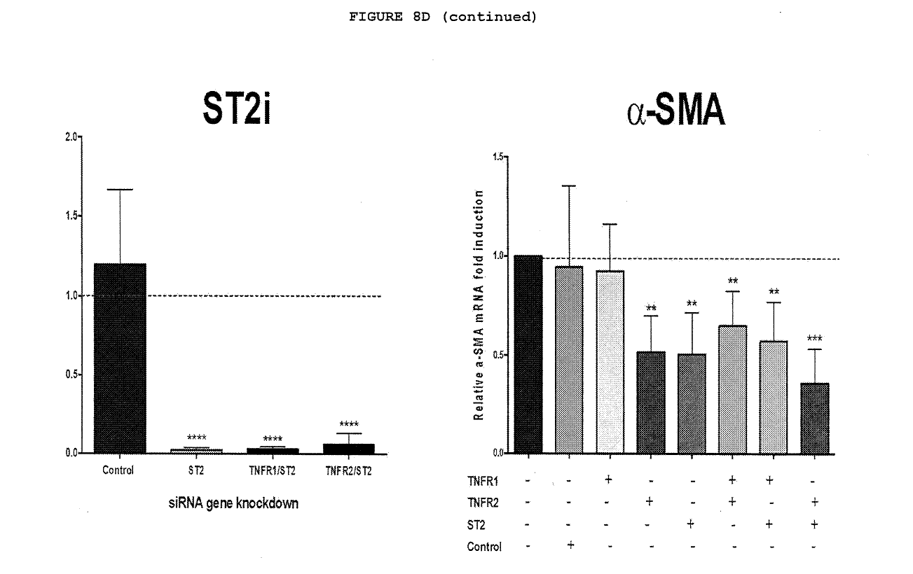

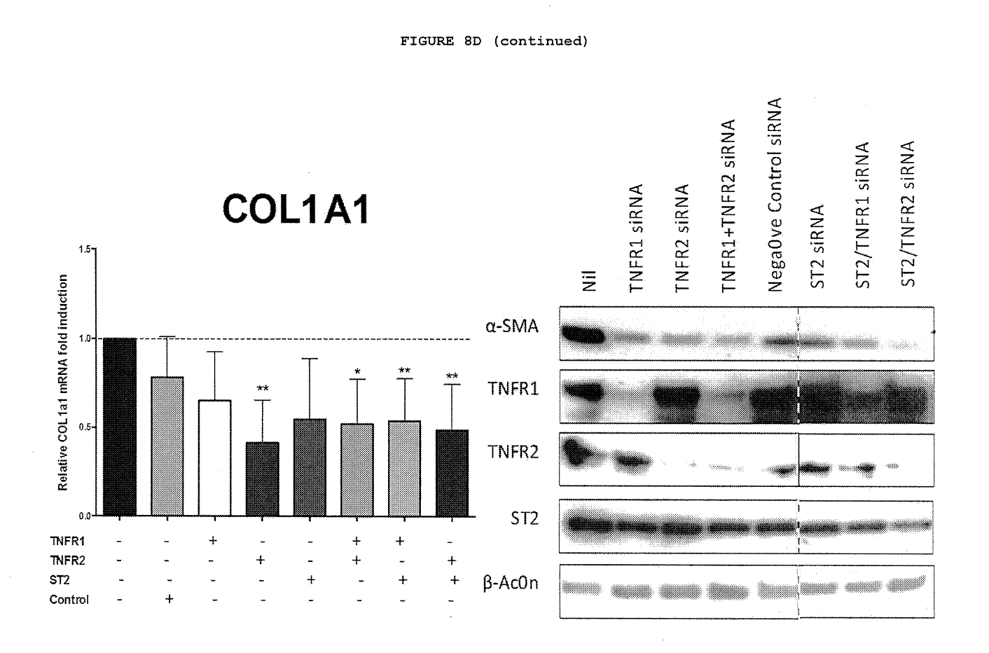

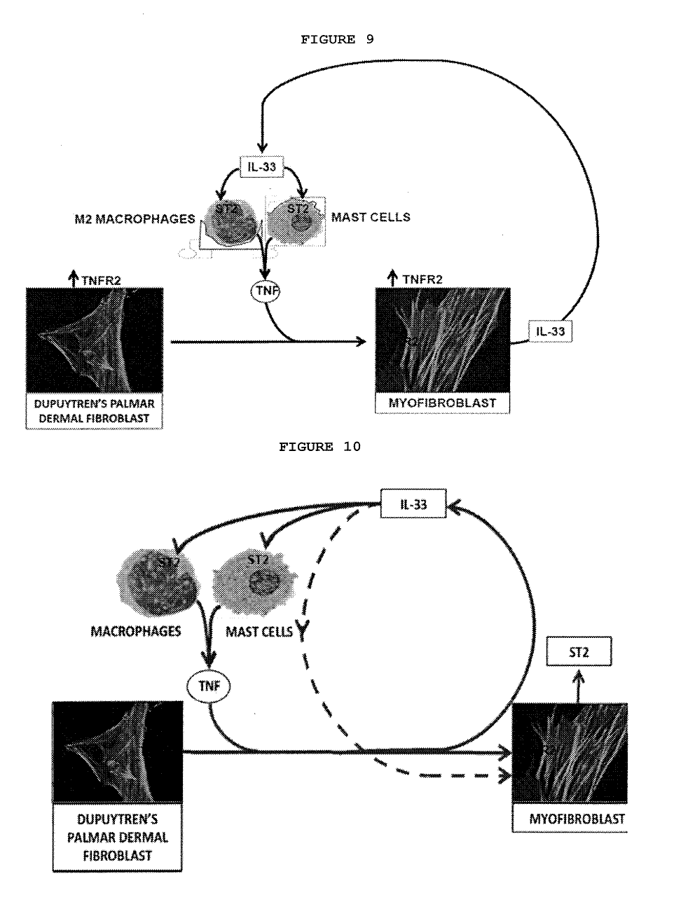

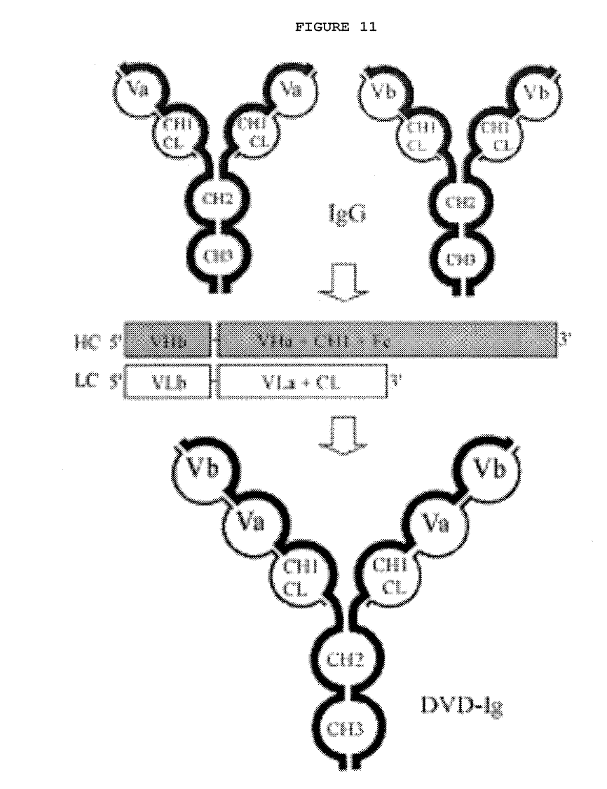

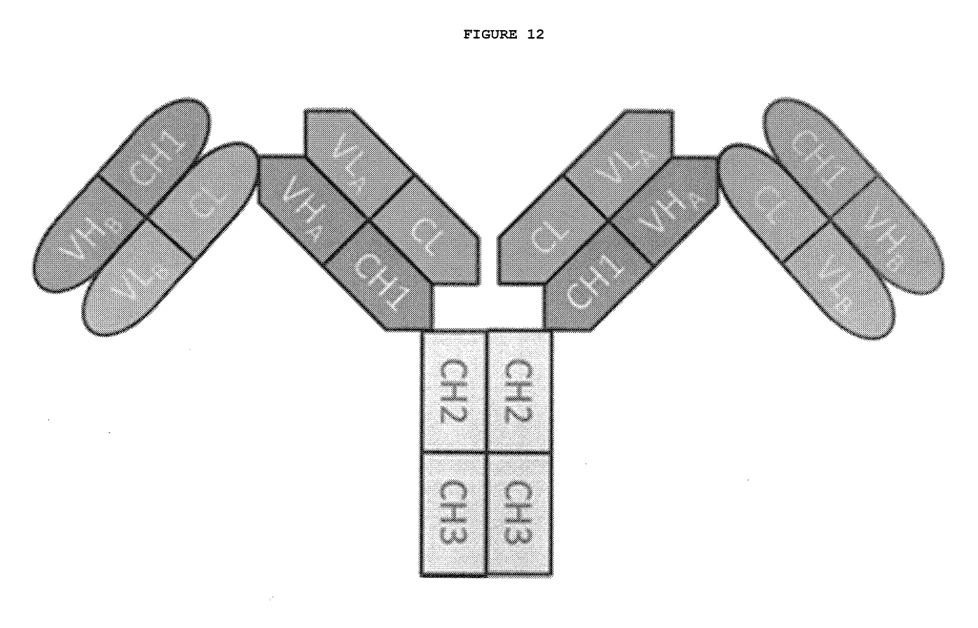

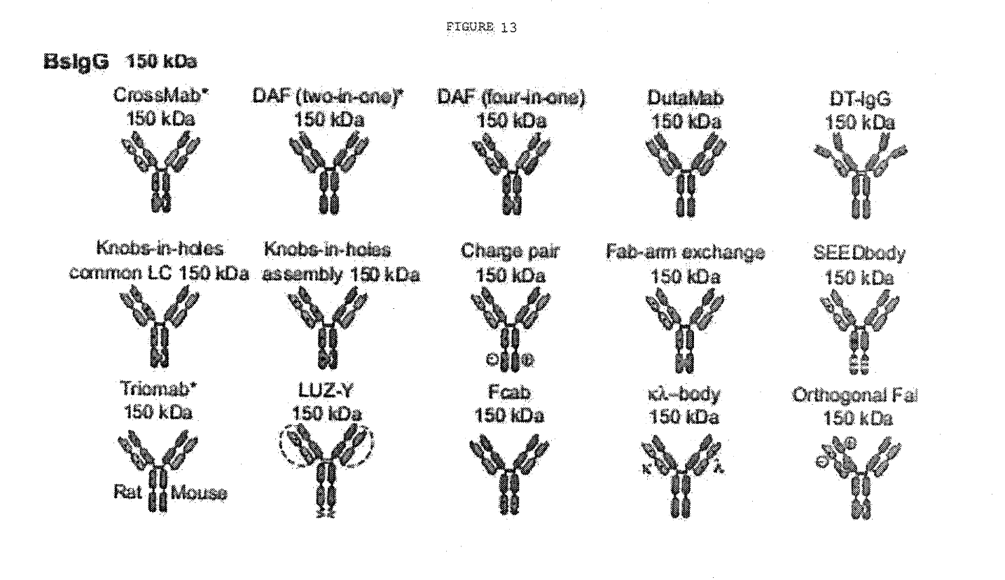

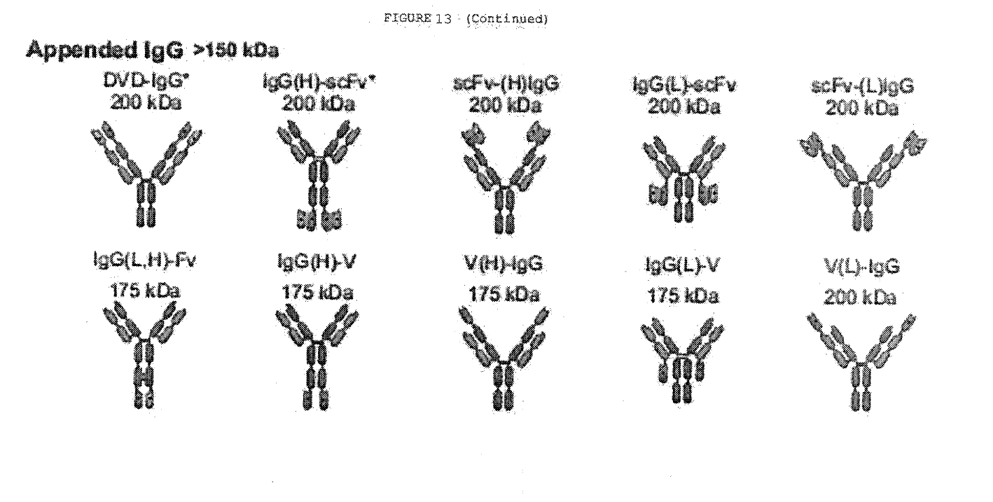

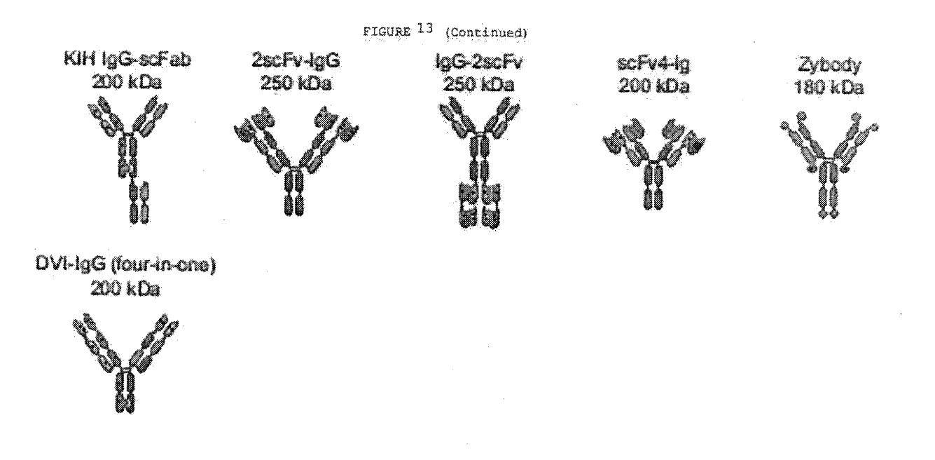

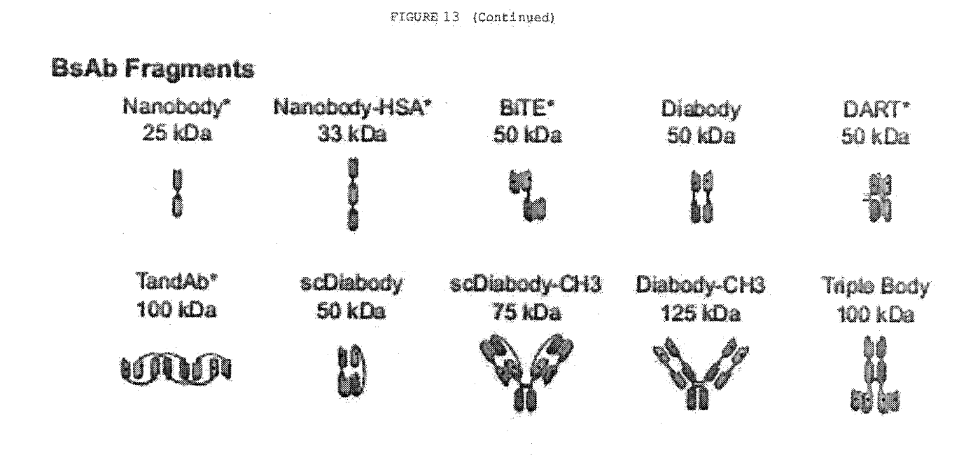

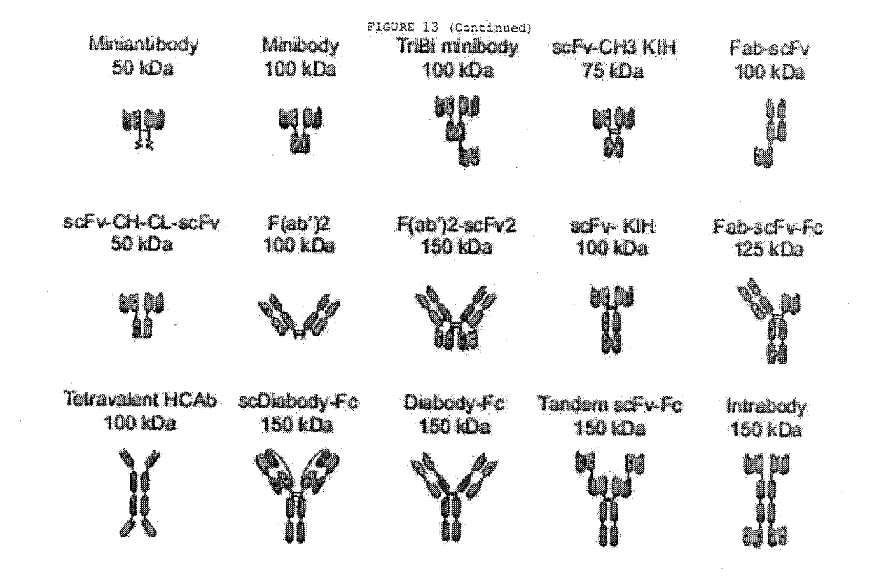

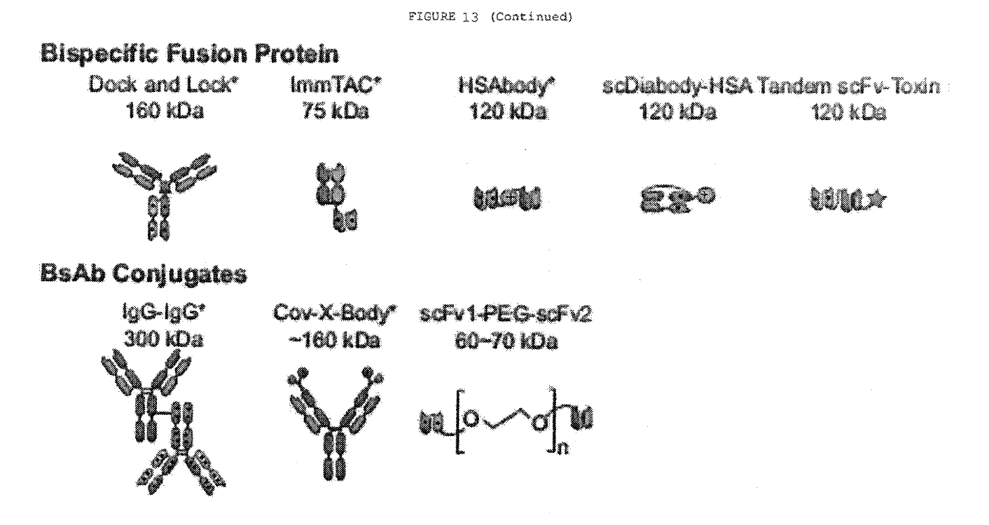

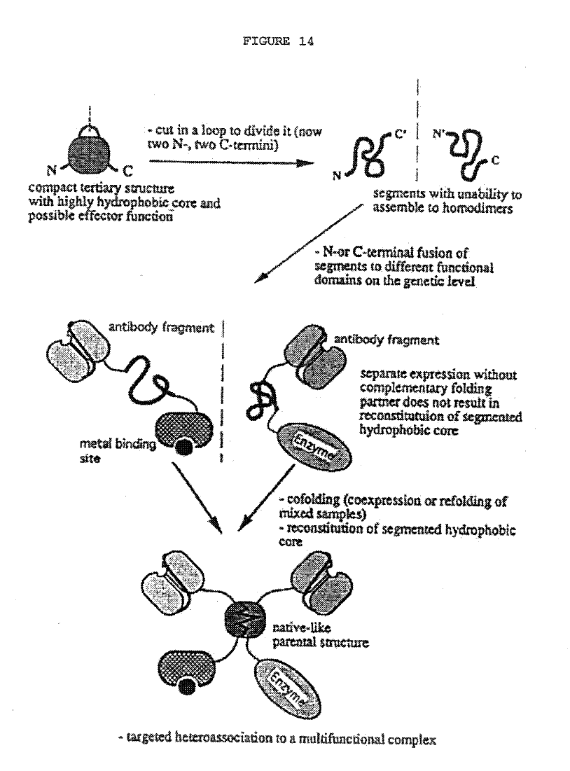

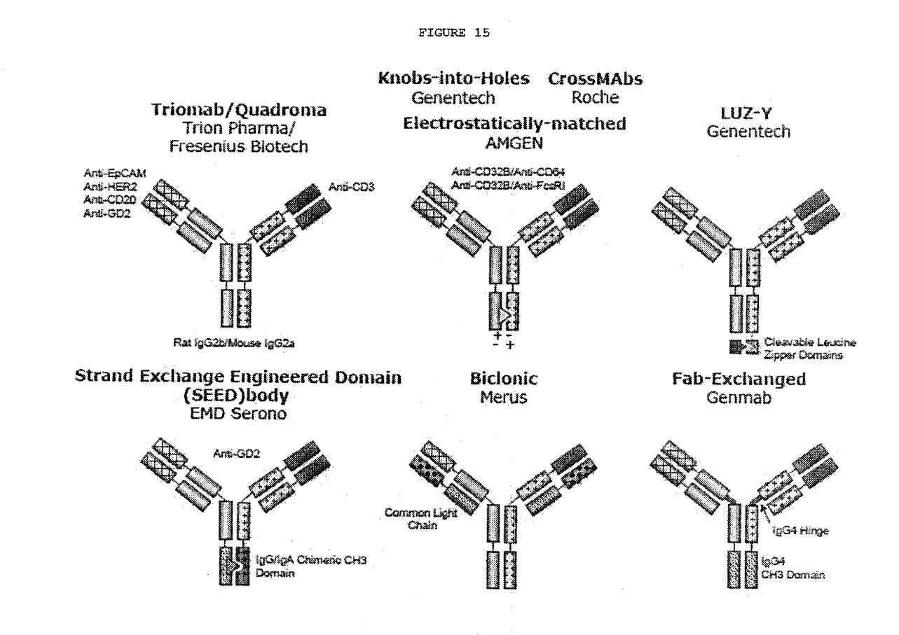

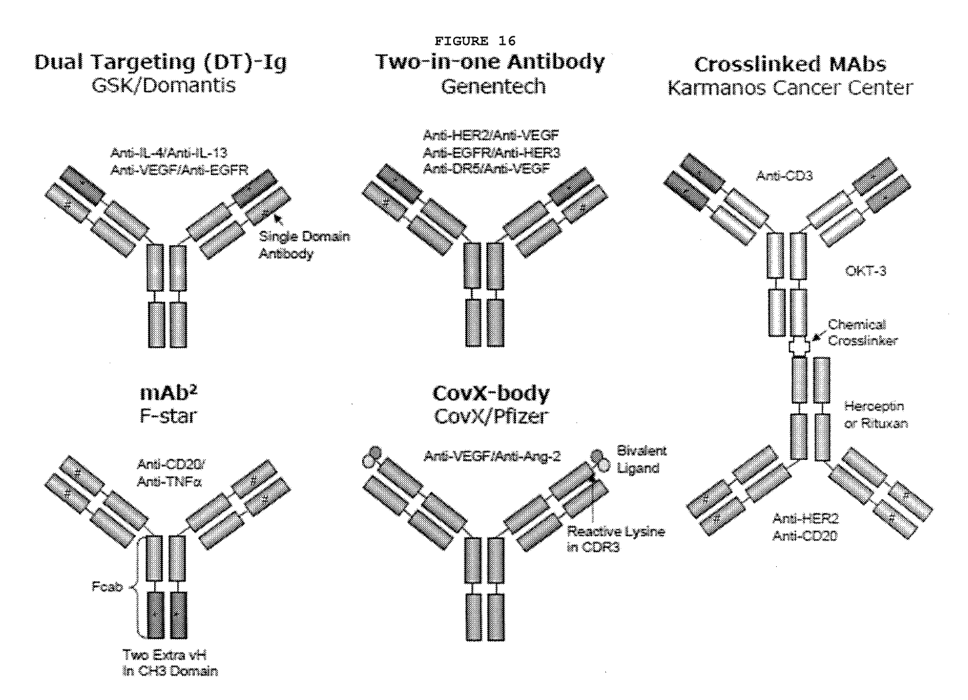

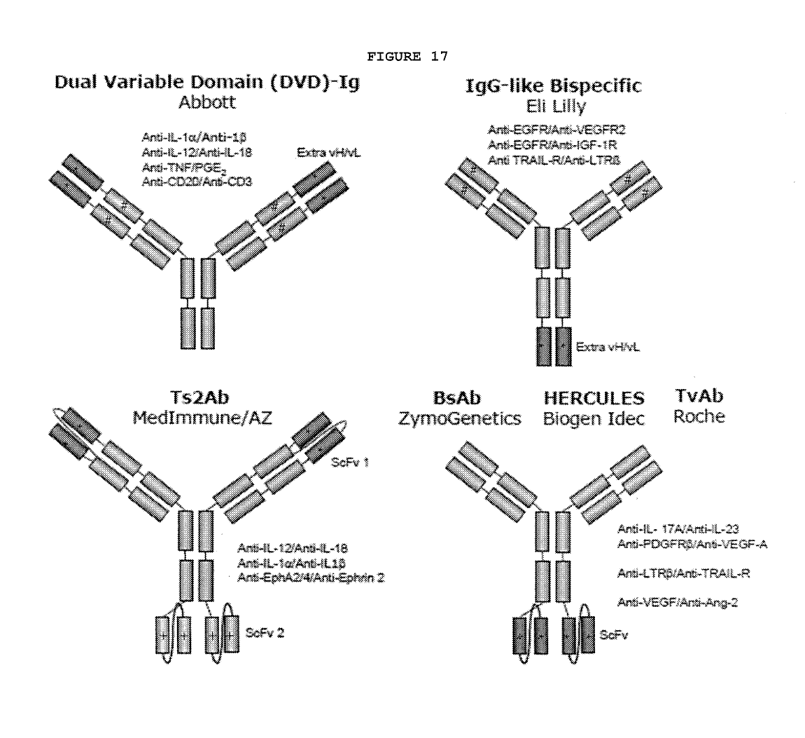

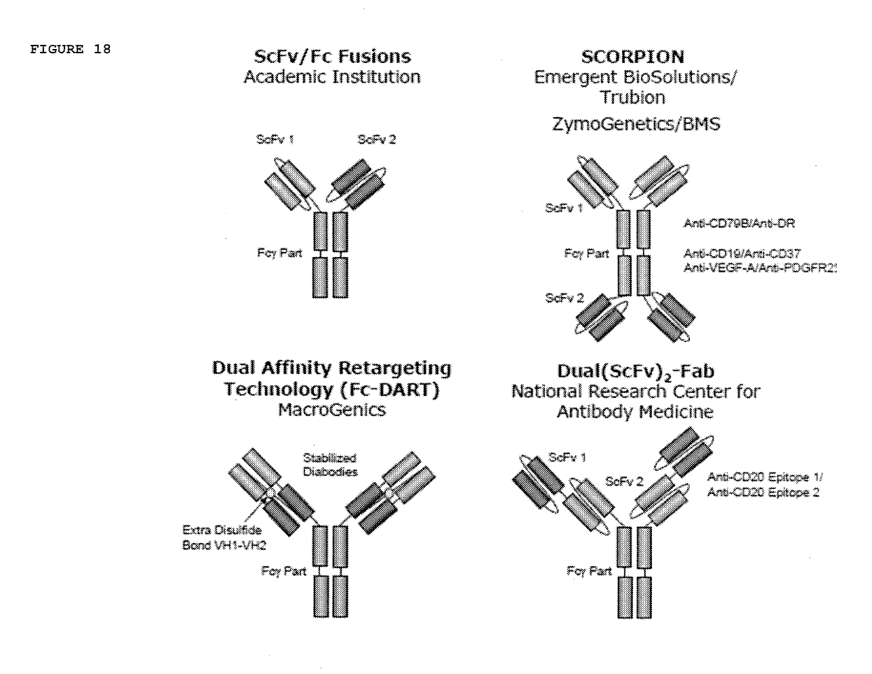

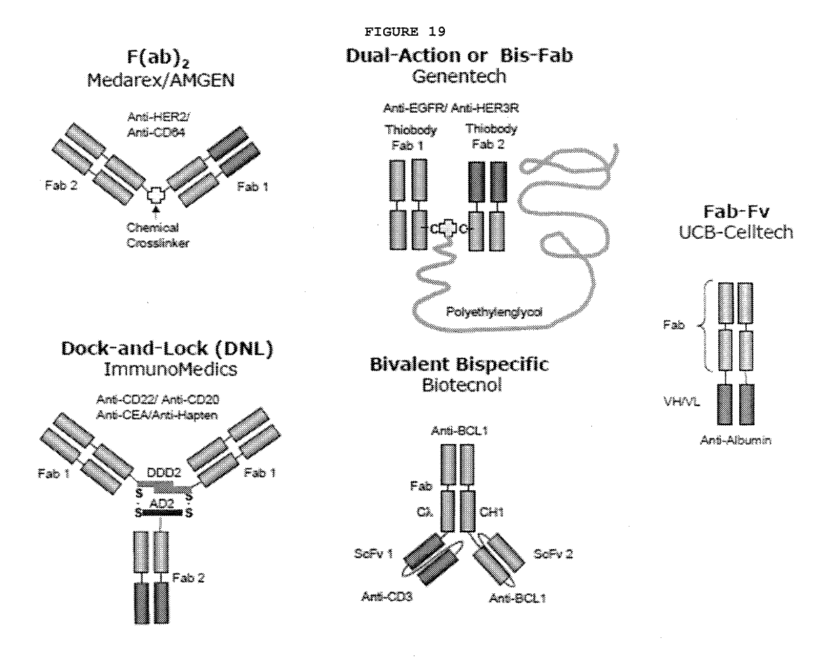

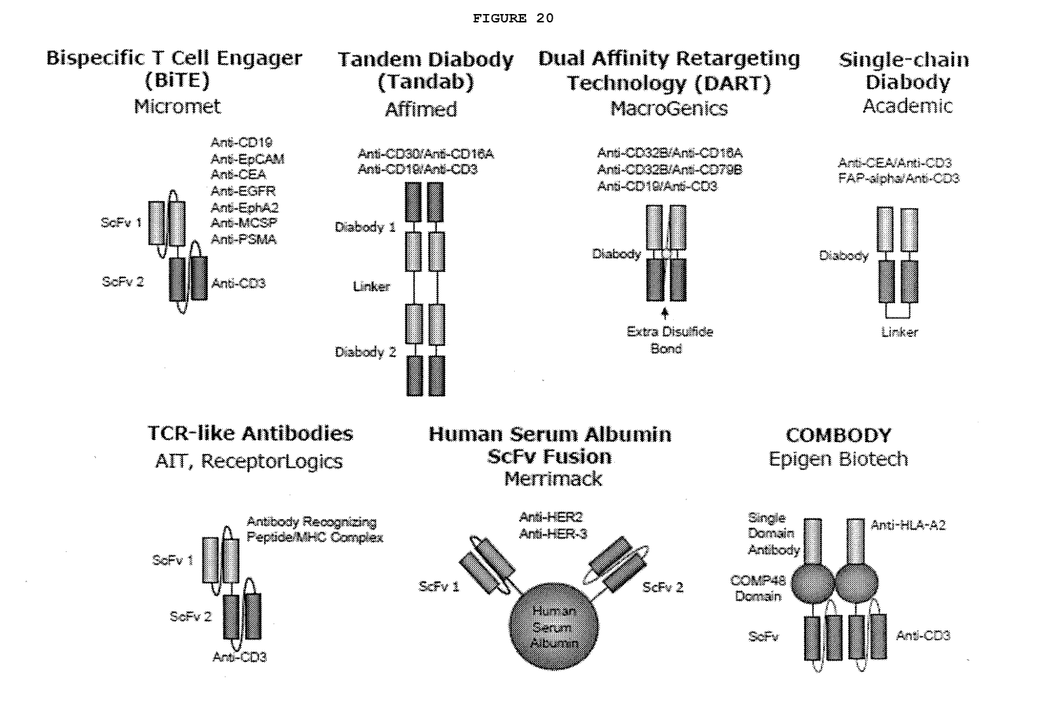

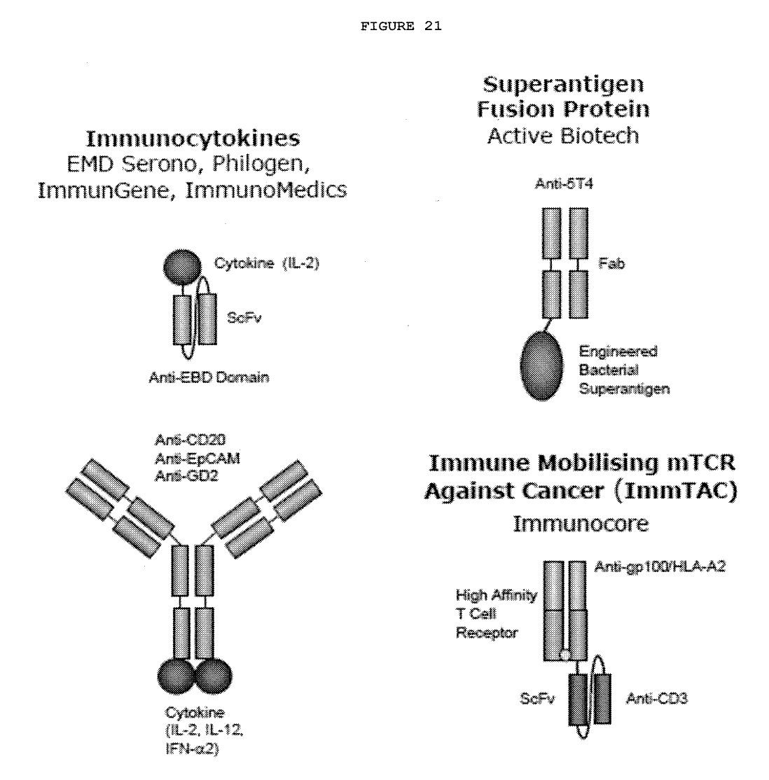

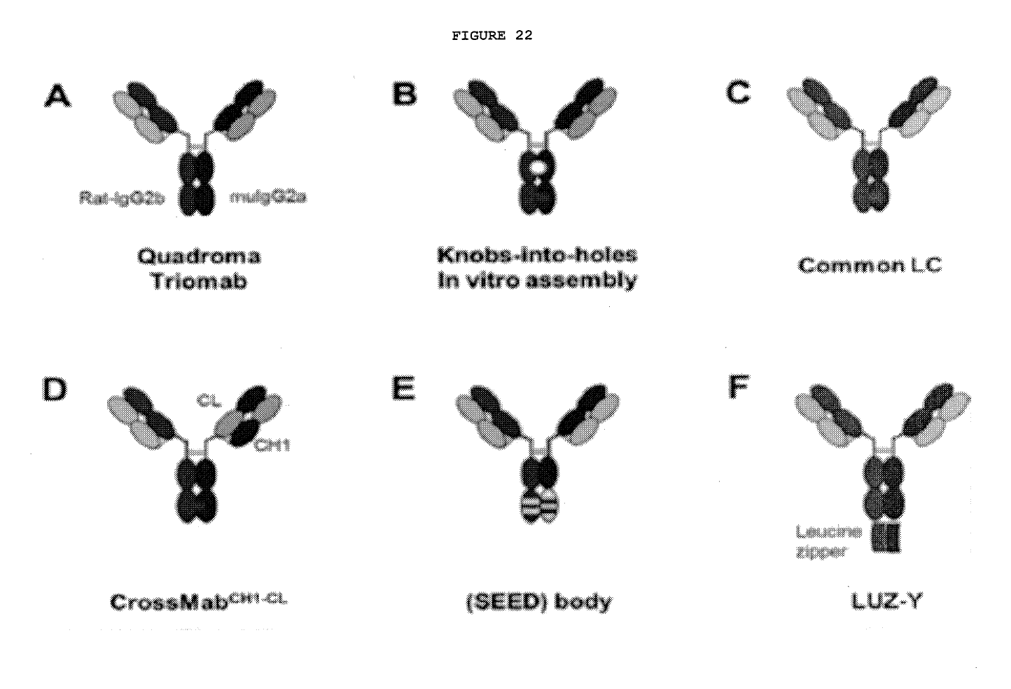

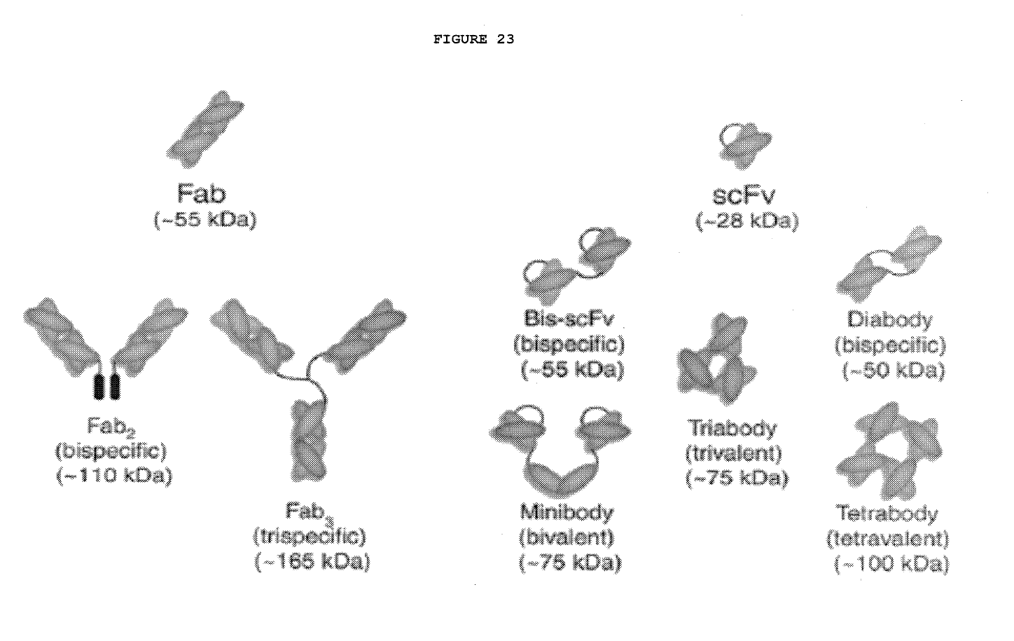

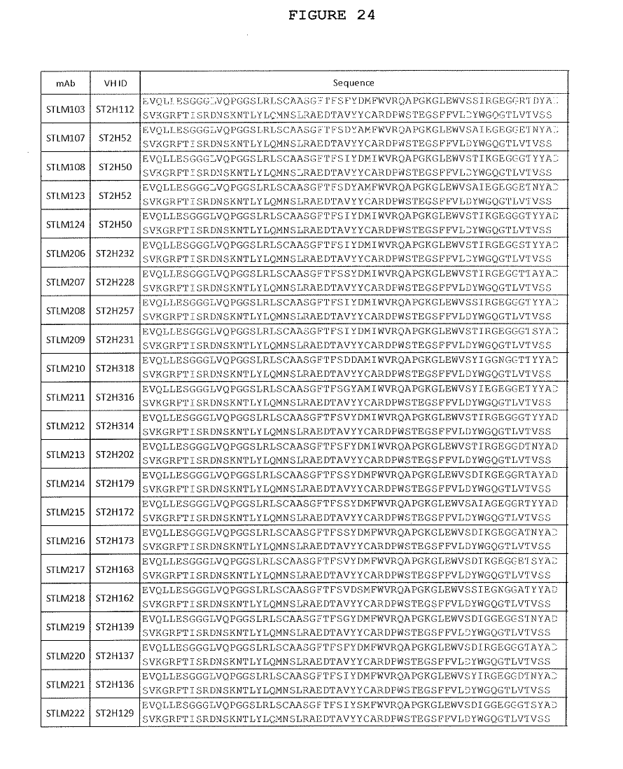

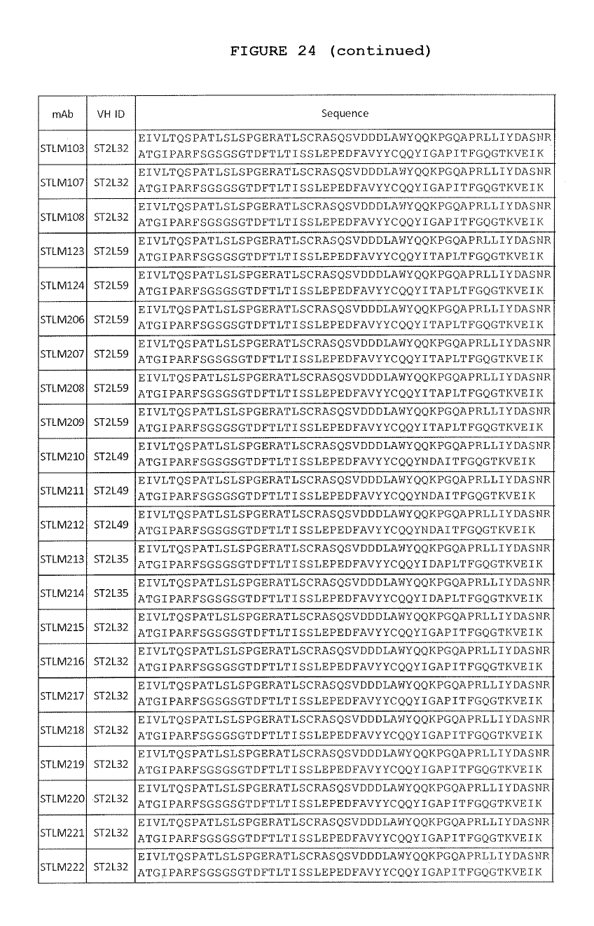

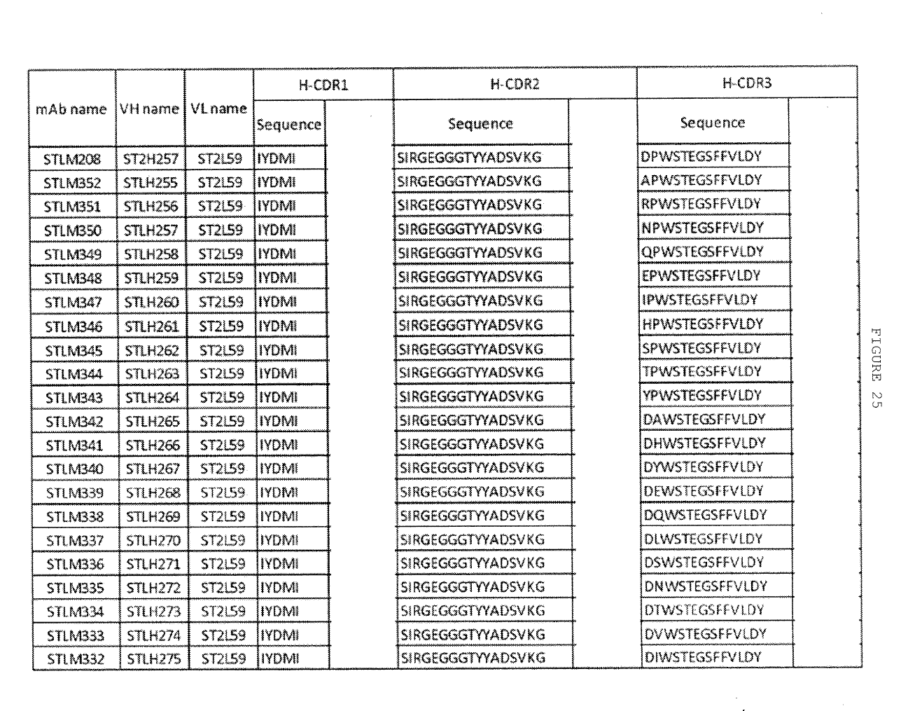

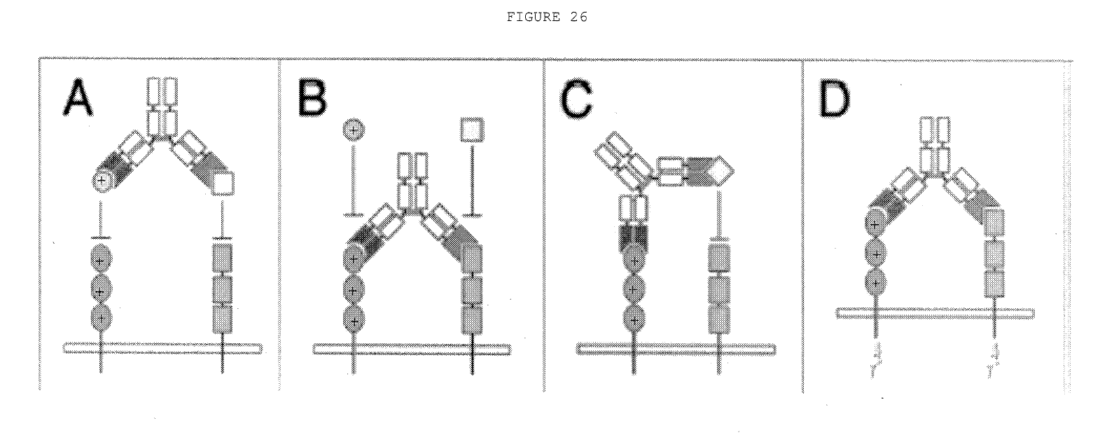

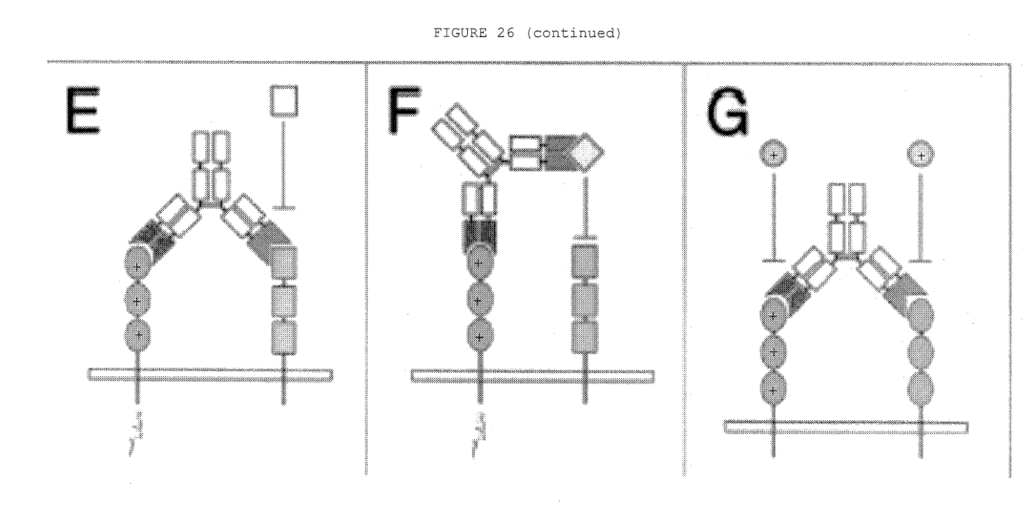

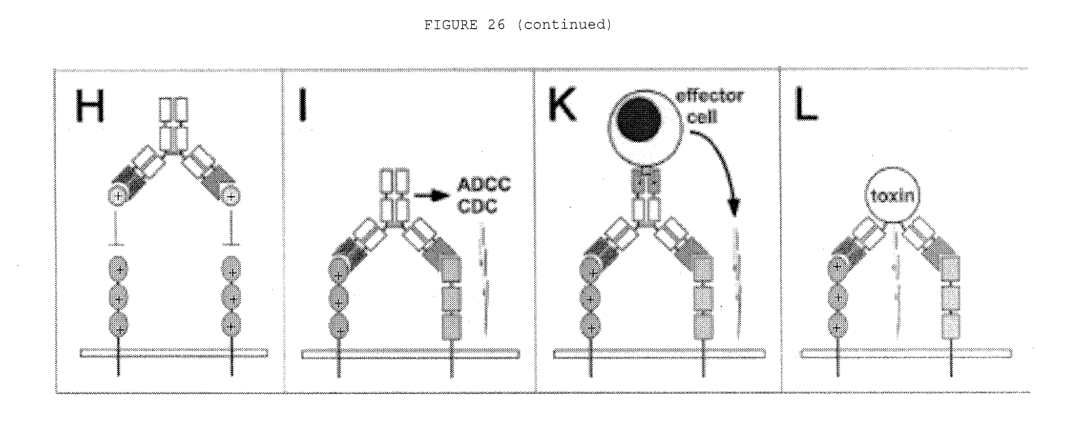

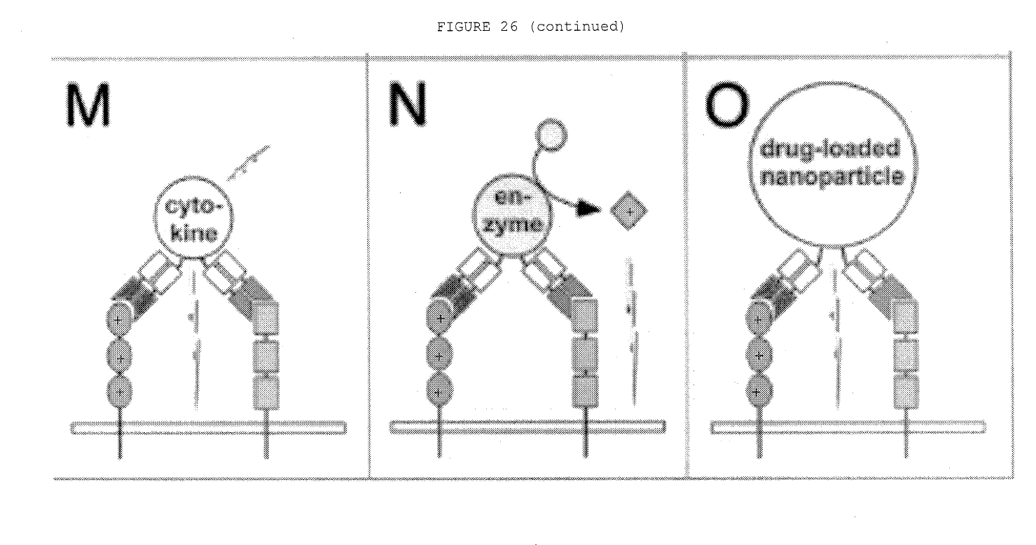

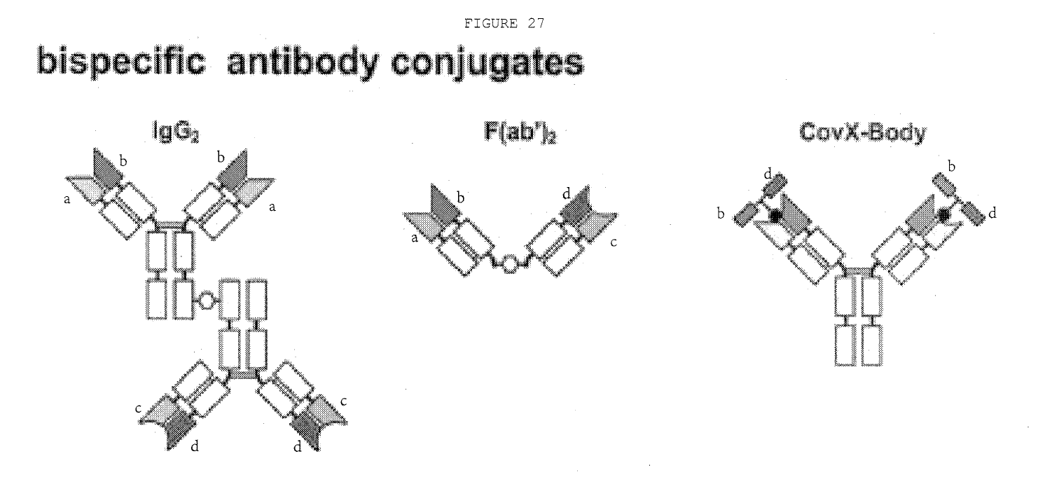

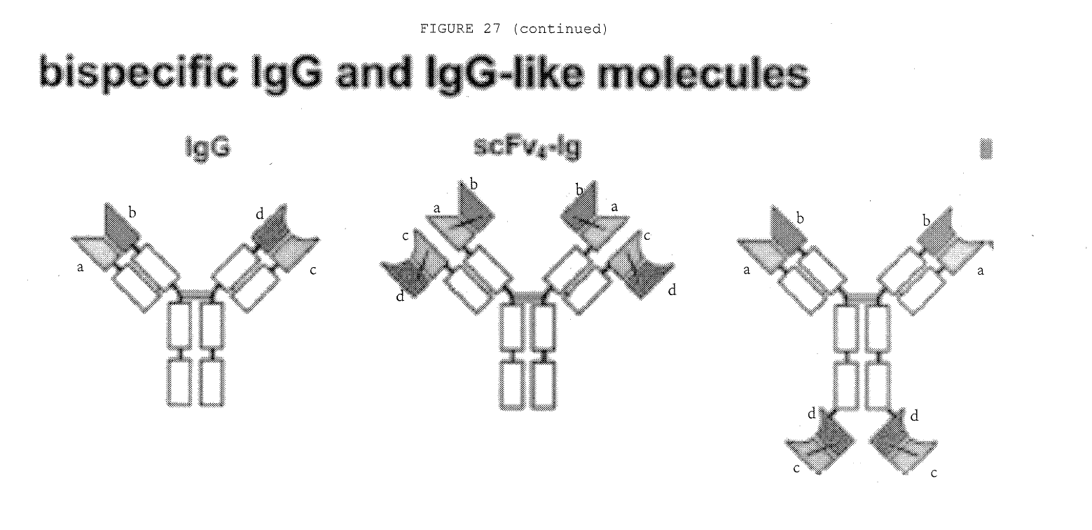

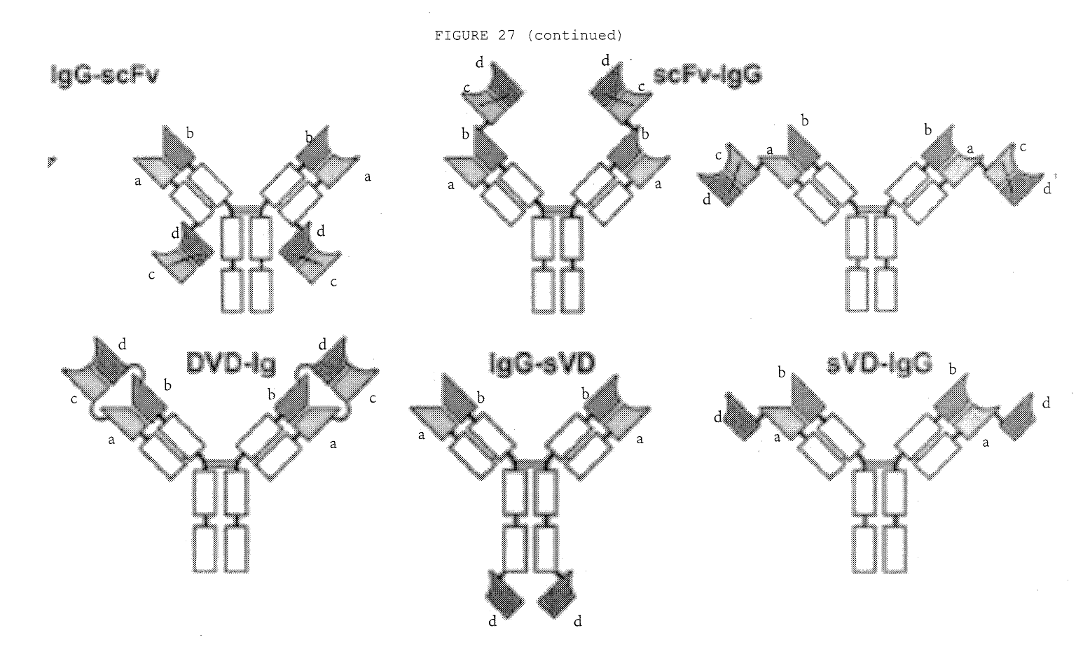

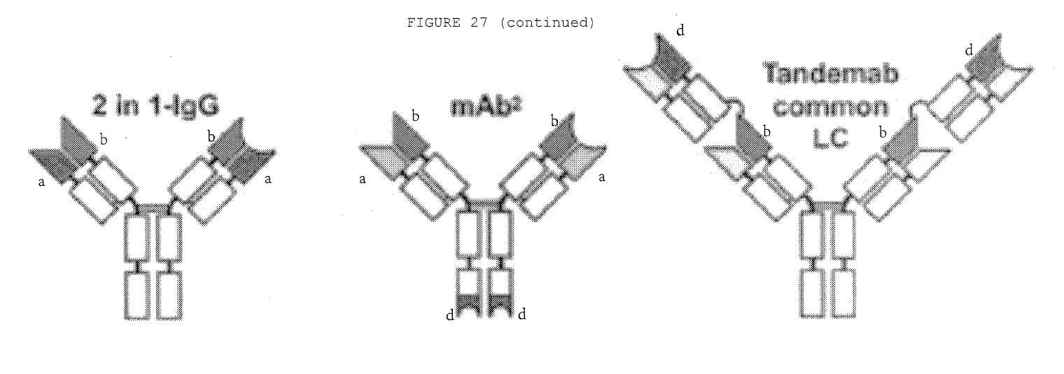

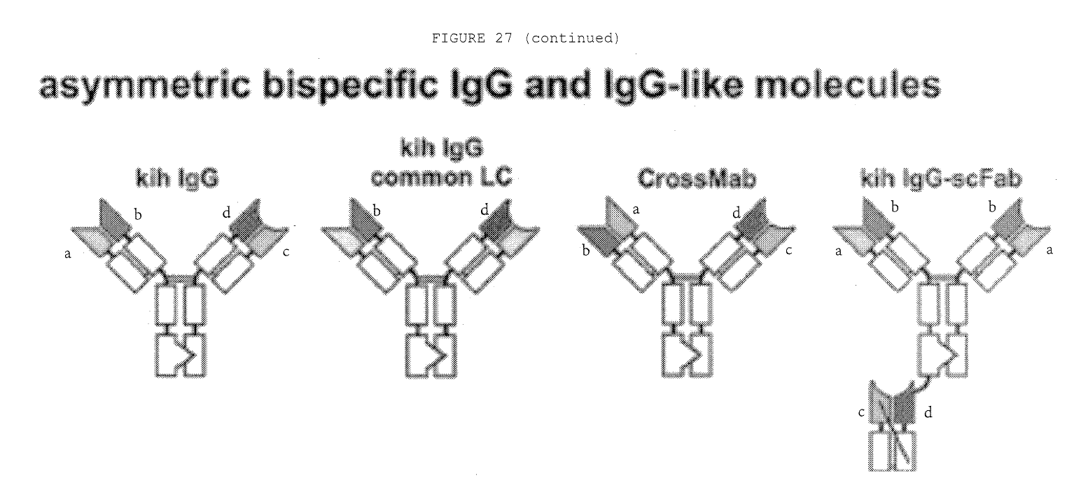

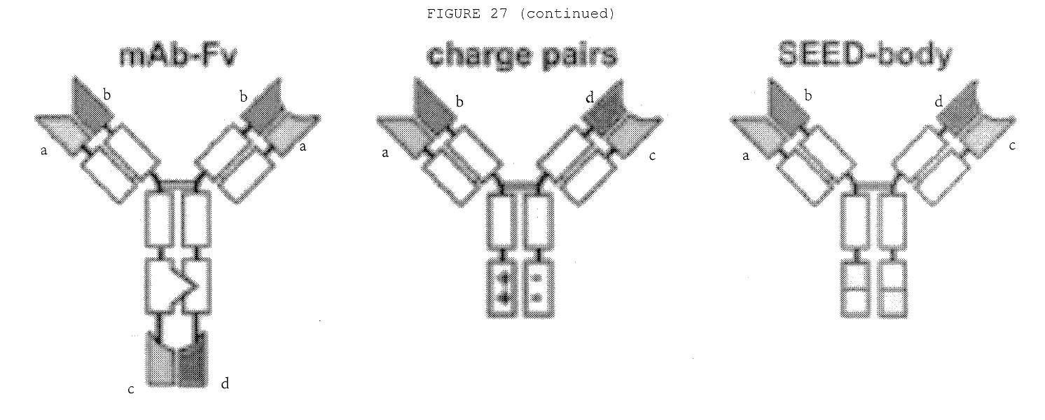

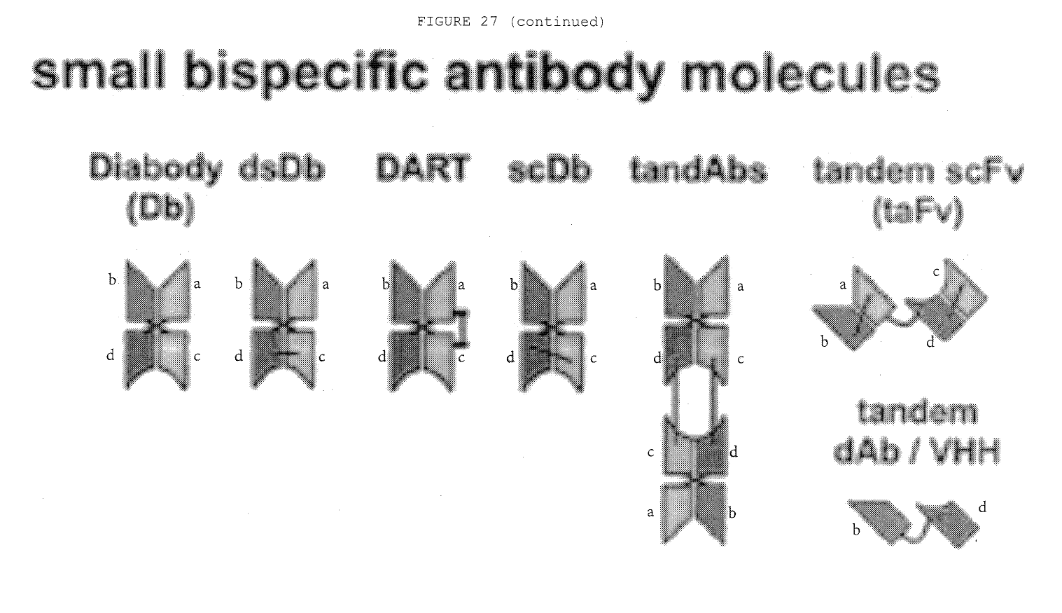

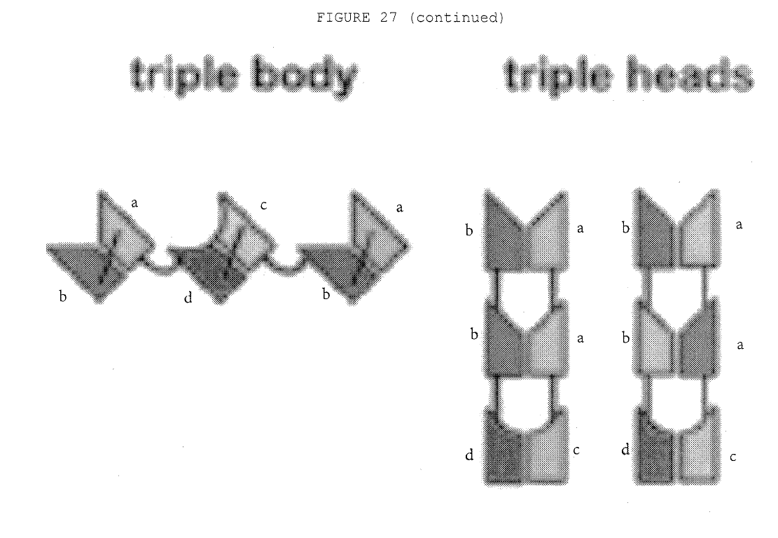

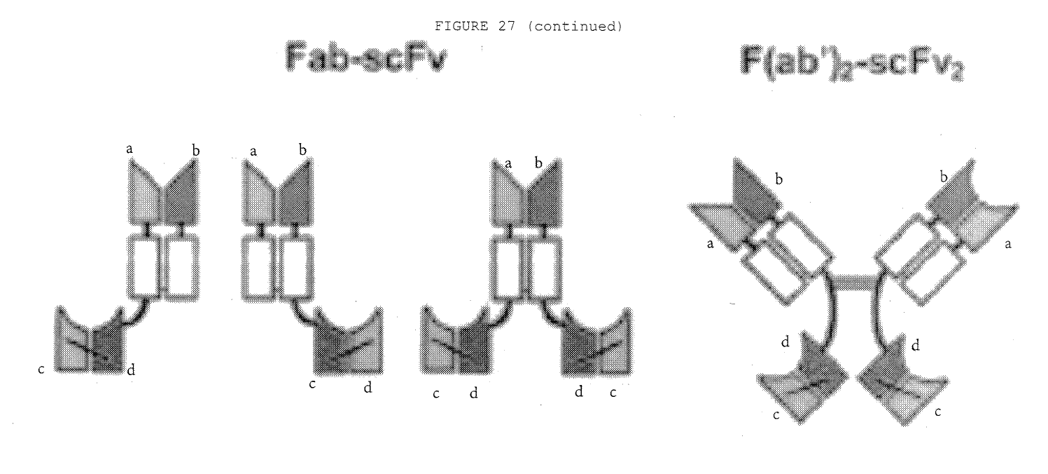

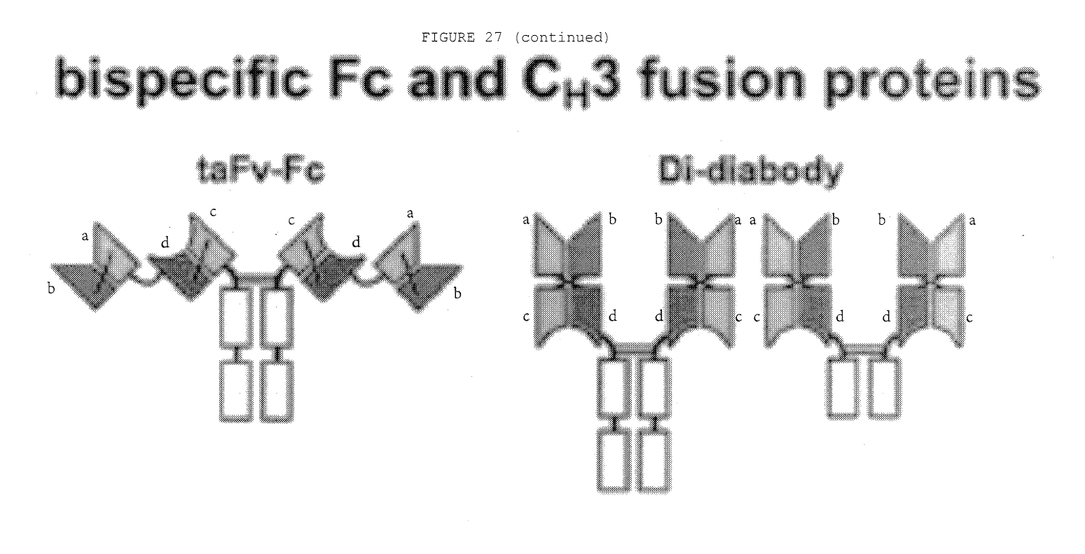

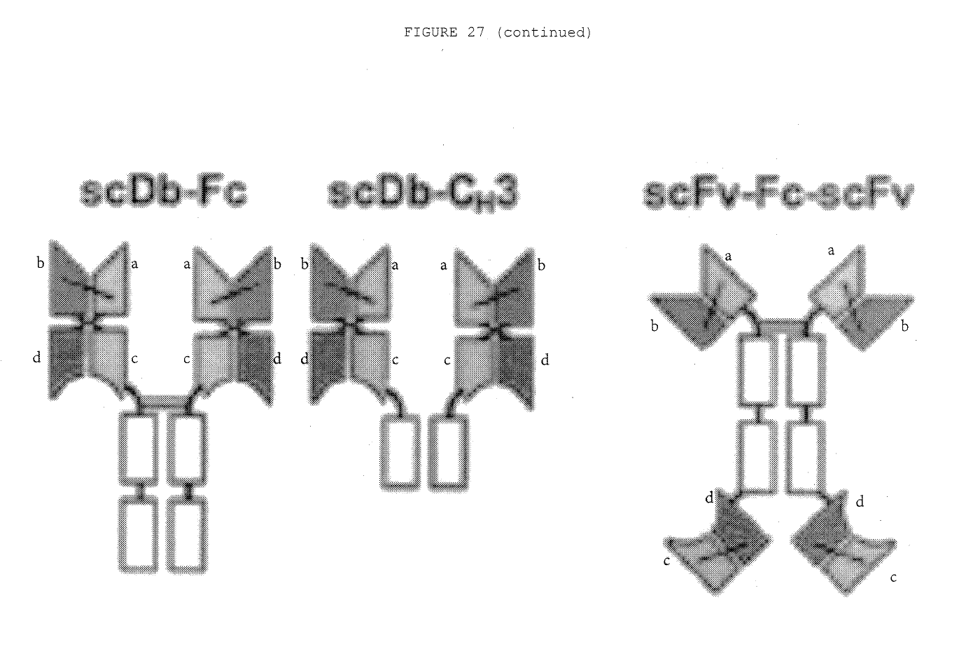

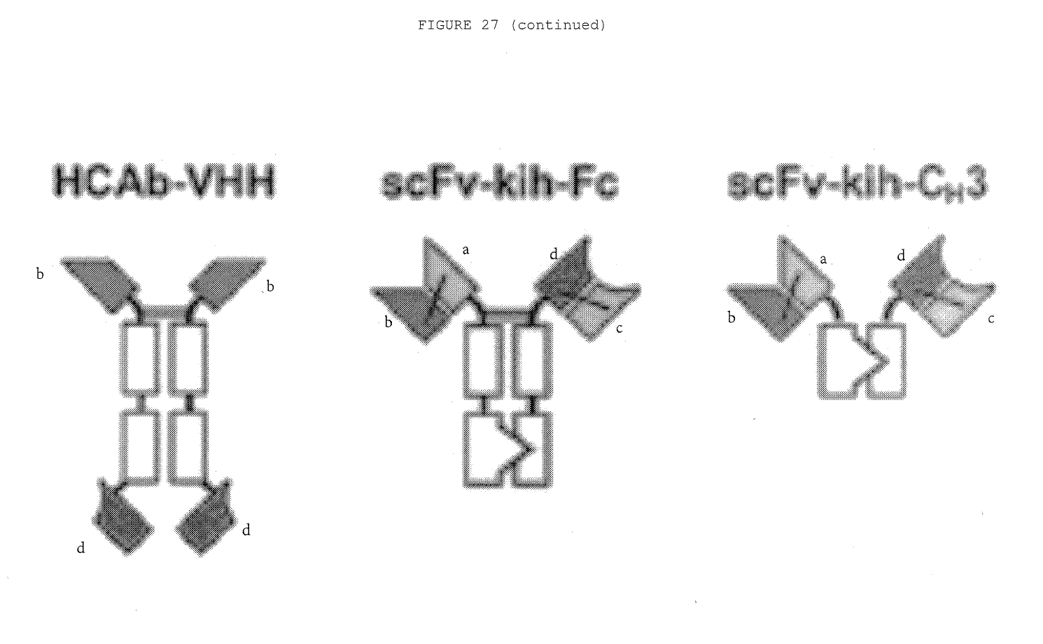

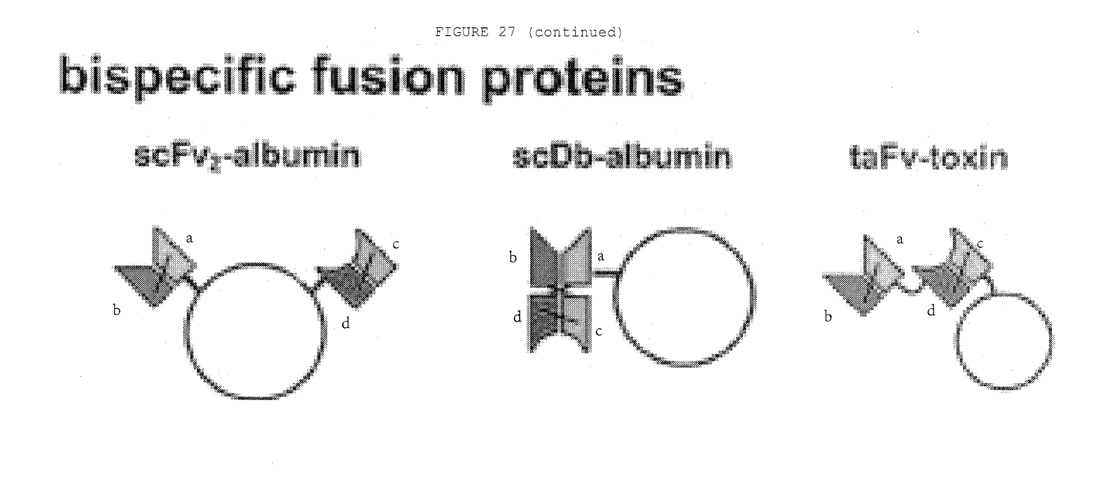

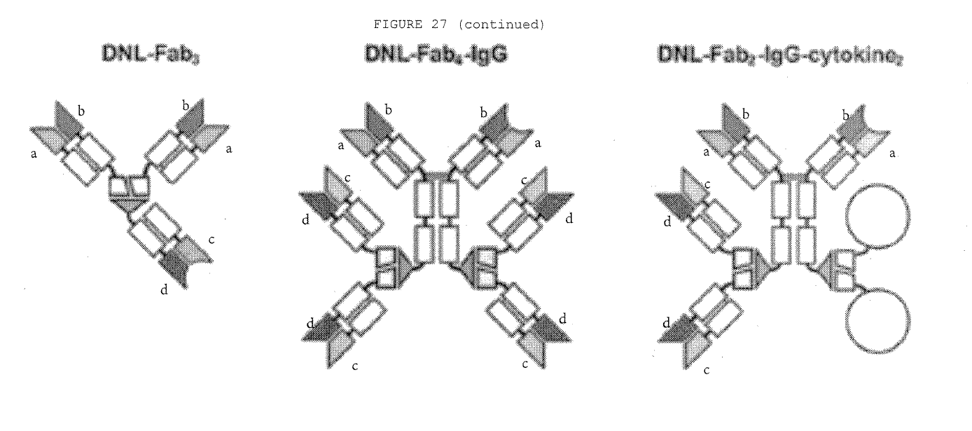

Analysis of supernatants from freshly disaggregated Dupuytren's tissue for a panel of cytokines and chemokines using Meso Scale Discovery (MSD) and detected IL-6 (FIG. 1), CCL2, CXCL8 (IL-8), CXCL10, and CCL26 (FIG. 4C) are presented in FIGS. 1 and 4C. The latter three are known chemokines for mast cells, which also require IL-6 as a growth factor. Elevated CXCL10 and CCL2 levels are consistent with the preponderance of classically activated M1 macrophages found in the Dupuytren's tissue (FIG. 1). These data suggest that macrophages and mast cells may be attracted to the Dupuytren's tissue by locally produced chemokines.

IL-33 was detected in the supernatant of freshly disaggregated Dupuytren's tissue (13.07±10.32 μg/ml) as shown in FIG. 1.

The best characterized human mast cell lines are LAD2, HMC1.1 and HMC1.2. Exposure of all three cell lines to recombinant human IL-33 (rhIL-33) resulted in a dose dependent secretion of TNF (FIG. 6D). Concentrations of IL-33 of the order released by freshly disaggregated tissue (10 pg/ml) led to TNF production at concentrations similar to those secreted ex vivo by freshly disaggregated cells from Dupuytren's nodules (Verjee, 2013).

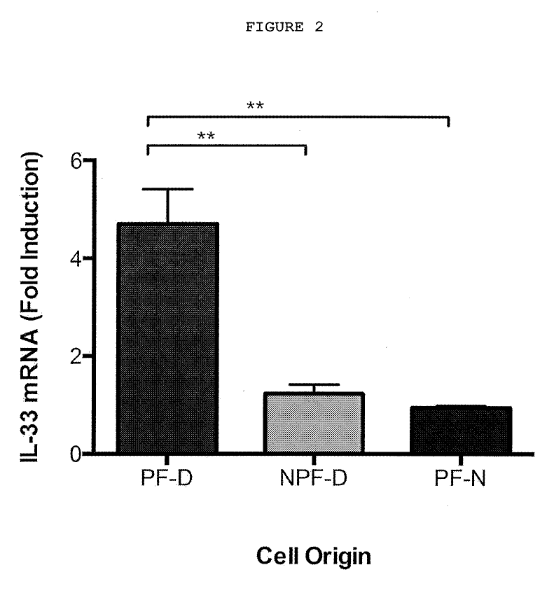

Only palmar dermal fibroblasts (PF-D) from patients with Dupuytren's disease expressed IL-33 on exposure to TNF (FIG. 2) while TGF-β1 indiscriminately induced expression of IL-33 in both palmar and non-palmar dermal fibroblasts (NPF-D) from these patients and in dermal fibroblasts from normal non-Dupuytren's controls (PF-N).

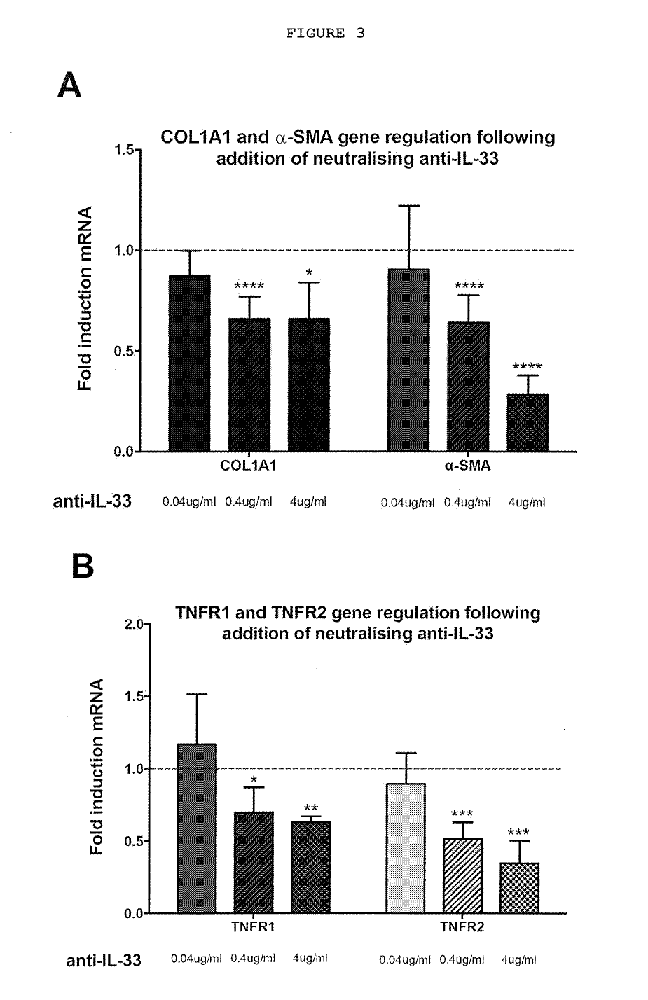

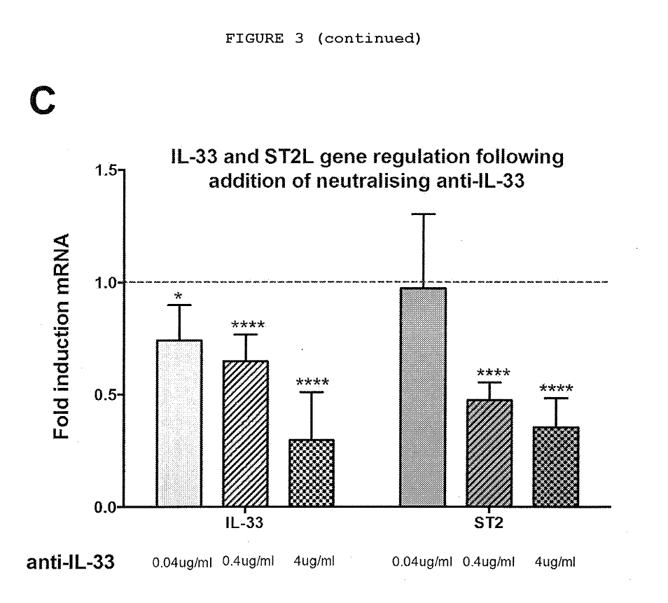

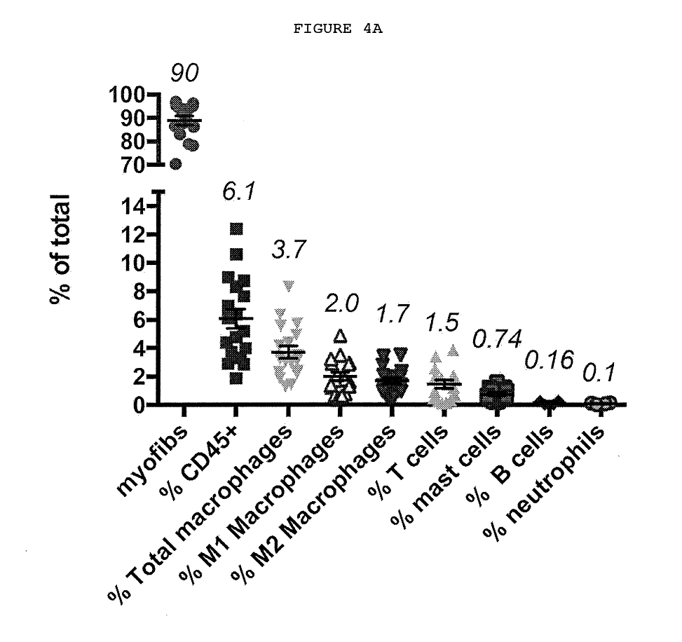

Treatment with anti-IL-33 resulted in a downregulation of the myofibroblasts phenotype in a dose-dependent manner (FIG. 3A). Inhibition of IL-33 also resulted in reduction of IL-33 and ST2 (a receptor for IL-33) expression by myofibroblasts, again in a dose-dependent manner (FIG. 3C). The interaction between the IL-33 and TNF pathways was confirmed as anti-IL-33 resulted in reduced expression of the receptors for TNF, TNFR1 and TNFR2 (FIG. 3B).

FIG. 8C notably and unexpected demonstrates that 82% of patients responded to anti-TNF and anti-IL-33. Additionally, it was also unexpectedly shown that 100% of patients respond to anti-TNFR2 and anti-IL-33. Therefore, applicants have shown that targeting TNFR2 and IL-33 is superior and advantageous compared to using anti-TNF and anti-IL-33. This could not have been predicted and was unexpected. IL-33 stimulates TNF production by classically activated macrophages and mast cells recruited during fibrosis. Elevated local levels of TNF lead to the synthesis of more IL-33 by palmar fibroblasts as they differentiate into myofibroblasts. This in turn promotes further TNF production, creating a positive feedback loop and a chronic fibrotic response. Furthermore, IL-33 enhances the expression of its ST2 receptor on myofibroblasts, thereby inducing a positive autocrine feedback loop (FIG. 10).

### Example 2

It is contemplated that periodically administering a therapeutically effective amount of an IL-33 antagonist to a patient suffering from Dupuytren's disease successfully treats such a patient.

### Example 3

It is contemplated that periodically administering a therapeutically effective amount of an IL-33 antagonist to a patient suffering from frozen shoulder successfully treats such a patient.

### Example 4

It is contemplated that periodically administering a therapeutically effective amount of an IL-33 antagonist to a patient suffering from periarticular fibrosis successfully treats such a patient.

### Example 5

It is contemplated that periodically administering a therapeutically effective amount of an IL-33 antagonist to a patient suffering from keloid or hypertrophic scars successfully treats such a patient.

### Example 6

It is contemplated that periodically administering a therapeutically effective amount of an IL-33 antagonist to a patient suffering from endometriosis successfully treats such a patient.

### Example 7

It is contemplated that periodically administering a therapeutically effective amount of an IL-33 antagonist to a patient suffering from abdominal adhesions successfully treats such a patient.

### Example 8

It is contemplated that periodically administering a therapeutically effective amount of an IL-33 antagonist to a patient suffering from Ledderhose disease successfully treats such a patient.

### Example 9

It is contemplated that periodically administering a therapeutically effective amount of an IL-33 antagonist to a patient suffering from Peyronie's disease successfully treats such a patient.

### Example 10

It is contemplated that periodically administering a therapeutically effective amount of an IL-33 antagonist to a patient suffering from peritendinous adhesions successfully treats such a patient.

### Example 11

It is contemplated that periodically administering a therapeutically effective amount of an IL-33 antagonist to a patient suffering from periarticular fibrosis successfully treats such a patient.

### Example 12

It is contemplated that co-administering a therapeutically effective amount of an IL-33 antagonist and a therapeutically effective amount of a TNF antagonist to a patient suffering from Dupuytren's disease successfully treats such a patient.

### Example 13: Add-on Therapy for Treating Dupuytren's Disease

It is contemplated that periodically administering an IL-33 antagonist as an add-on therapy for a human patient afflicted with Dupuytren's disease who is already receiving an TNF antagonist provides a clinically meaningful advantage and is more effective (provides at least an additive effect or more than an additive effect) in treating the patient than when the TNF antagonist is administered alone (at the same dose).

It is contemplated that periodically administering a TNF antagonist as an add-on therapy for a human patient afflicted with Dupuytren's disease who is already receiving an IL-33 antagonist provides a clinically meaningful advantage and is more effective (provides at least an additive effect or more than an additive effect) in treating the patient than when the IL-33 antagonist or the bispecific antibody is administered alone (at the same dose).

The add-on therapies also provides efficacy (provides at least an additive effect or more than an additive effect) in treating the patient without undue adverse side effects or affecting the safety of the treatment:

1. The add-on therapy is effective (provides at least an additive effect or more than an additive effect) in improving nodule size.
2. The add-on therapy is effective (provides at least an additive effect or more than an additive effect) in improving grip strength and range of motion of the affected digit.

### Example 14: Combination Therapy for Treating Dupuytren's Disease

Disclosed herein is the use of an IL-33 antagonist in addition to or in combination with a TNF antagonist for the treatment of Dupuytren's disease.

It is contemplated that periodically administering a IL-33 antagonist in combination with a TNF antagonist or a IL-33/TNF bispecific antibody to a human patient afflicted with Dupuytren's disease provides increased efficacy (provides at least an additive effect or more than an additive effect) in treating the patient than when a TNF antagonist is administered alone or when an IL-33 antagonist is administered alone (at the same dose). The combination therapy also provides efficacy (provides at least an additive effect or more than an additive effect) in treating the patient without undue adverse side effects or affecting the safety of the treatment.

The combination therapy provides a clinically meaningful advantage and is more effective (provides at least an additive effect or more than an additive effect) in treating the patient than when the IL-33 antagonist or a TNF antagonist is administered alone (at the same dose) in the following manner:

1. The combination therapy is effective (provides at least an additive effect or more than an additive effect) in improving nodule size and vascularity.
2. The combination therapy is effective (provides at least an additive effect or more than an additive effect) in improving grip strength and range of motion of the affected digit.
3. The combination therapy reduces the frequency of injections needed to treat Dupuytren's disease.
4. The combination therapy is effective in (provides at least an additive effect or more than an additive effect) treating a greater percentage of patients than either the IL-33 antagonist alone or the TNF antagonist alone.
5. The combination therapy delays the progression of early disease stage.

### Example 15: Inhibition of Expression of TNFR2, ST2 and Most Effectively TNFR2+ST2 Down Regulates Myofibroblast Phenotype (FIG. 8D)

Methods for siRNA.

Cultured myofibroblasts from patients with Dupuytren's disease were used up to passage 2. 400,000 cells were mixed with 100 μl of Nucleofector Kit for Human Dermal Fibroblast transfection reagent (VPD-1001, Lonza) and 60 nM silencer select siRNA (Applied Biosystem), then electroporated using the AMAXA nucleofection 2b Device (Lonza) to transfect the siRNA probes. Inventoried silencer-select reagents and respective non-targeting negative controls were used for TNFR1 (4390824s, siRNA ID s14265), TNFR2 (439420, siRNA ID s14270), IL1RL1 (439420, s17532, Applied Biosystems).

TNFR2 sense 5′ to 3:
(SEQ ID NO: 1)
GCCUUGGGUCUACUAAUAATT.
TNFR1 sense 5′ to 3:
(SEQ ID NO: 2)
CGGUGACUGUCCCAACUUUTT.
IL1RL1 sense 5′ to 3:
(SEQ ID NO: 3)
GUUACACCGUGGAUUGGUATT.

Negative control siRNAs 1(4390843) and 2 (4390846) (Applied Biosystems) were used with sequences that do not target any gene product and provide a baseline to compare siRNA-treated samples. Cells were immediately transferred to a 6-well plate with 2 ml OptiMEM (31985062, Life Technologies) without serum, pre-warmed to 37° C. in an incubator with 5% CO2. After 16 h the transfection medium was washed three times with Phosphate Buffered Saline, before being replaced by DMEM with 10% FBS and 1% penicillin/streptomycin and incubated for another 32 h in a 37° C. incubator with 5% CO2. RT-PCR analysis was used to quantify knockdown of gene as previously described.

### Discussion and Results:

TNFR1 expression is effectively down regulated by siRNA knockdown of TNFR1, TNFR1+TNFR2 or TNFR1+ST2 knockdown. TNFR2 expression is reduced by siRNA knockdown of TNFR2, TNFR1+TNFR2 or TNFR2+ST2 knockdown. ST2 expression is reduced by siRNA knockdown of ST2, TNFR1+ST2 or TNFR2+ST2 knockdown. Myofibroblast phenotype is down regulated as evidenced by α-SMA expression by siRNA knock down of TNFR2 (but not TNFR1) or ST2 and most effectively by siRNA knockdown of TNFR2+ST2 at mRNA and protein levels. Expression of COL1A1 mRNA, another marker of the myofibroblast phenotype, is reduced by siRNA knockdown of TNFR2 (but not TNFR1), ST2 or TNFR2+ST2 (FIG. 8D).

### Example 16

It is contemplated that co-administering a therapeutically effective amount of an IL-33 antagonist and a therapeutically effective amount of a TNFR2 antagonist to a patient suffering from Dupuytren's disease successfully treats the patient.

### Example 17: Add-on Therapy for Treating Dupuytren's Disease

It is contemplated that periodically administering an IL-33 antagonist as an add-on therapy for a human patient afflicted with Dupuytren's disease who is already receiving an TNFR2 antagonist provides a clinically meaningful advantage and is more effective (provides at least an additive effect or more than an additive effect) in treating the patient than when the TNFR2 antagonist is administered alone (at the same dose).

It is contemplated that periodically administering a TNFR2 antagonist as an add-on therapy for a human patient afflicted with Dupuytren's disease who is already receiving an IL-33 antagonist provides a clinically meaningful advantage and is more effective (provides at least an additive effect or more than an additive effect) in treating the patient than when the IL-33 antagonist is administered alone (at the same dose).

The add-on therapies also provides efficacy (provides at least an additive effect or more than an additive effect) in treating the patient without undue adverse side effects or affecting the safety of the treatment:

1. The add-on therapy is effective (provides at least an additive effect or more than an additive effect) in improving nodule size and vascularity.
2. The add-on therapy is effective (provides at least an additive effect or more than an additive effect) in improving grip strength and range of motion of the affected digit.

### Example 18: Combination Therapy for Treating Dupuytren's Disease

Disclosed herein is the use of an IL-33 antagonist in addition to or in combination with a TNFR2 antagonist for the treatment of Dupuytren's disease.

It is contemplated that periodically administering a IL-33 antagonist in combination with a TNFR2 antagonist to a human patient afflicted with Dupuytren's disease provides increased efficacy (provides at least an additive effect or more than an additive effect) in treating the patient than when a TNFR2 antagonist is administered alone or when an IL-33 antagonist is administered alone (at the same dose). The combination therapy also provides efficacy (provides at least an additive effect or more than an additive effect) in treating the patient without undue adverse side effects or affecting the safety of the treatment.

The combination therapy provides a clinically meaningful advantage and is more effective (provides at least an additive effect or more than an additive effect) in treating the patient than when the IL-33 antagonist or a TNFR2 antagonist is administered alone (at the same dose) in the following manner:

1. The combination therapy is effective (provides at least an additive effect or more than an additive effect) in improving nodule size and vascularity.
2. The combination therapy is effective (provides at least an additive effect or more than an additive effect) in improving grip strength and range of motion of the affected digit.
3. The combination therapy reduces the frequency of injections needed to treat Dupuytren's disease.
4. The combination therapy is effective in (provides at least an additive effect or more than an additive effect) treating a greater percentage of patients than either the IL-33 antagonist alone or the TNFR2 antagonist alone.
5. The combination therapy delays the progression of early disease stage.

### Example 19: Generation of a DVD-Ig

DVD-Ig molecules that bind two antigens are constructed using two parent monoclonal antibodies, one against TNF and the other against IL-33, selected as described herein.

### Example 20: Generation of a DVD-Ig Having Two Linker Lengths

A constant region containing μl Fc with mutations at 234, and 235 to eliminate ADCC/CDC effector functions is used. Four different anti-TNF/IL-33 DVD-Ig constructs are generated: 2 with short linker (SL) and 2 with long linker (LL), each in two different domain orientations: VA-VB-C and VB-VA-C(see Table below).

The linker sequences, derived from the N-terminal sequence of human Cl/Ck or CH1 domain, are as follows:

### For DVDAB Constructs:

light chain (if anti-A has λ)
Short linker:
QPKAAP;
Long linker:
QPKAAPSVTLFPP
light chain (if anti-A has κ)
Short linker:
TVAAP;
Long linker:
TVAAPSVFIFPP
heavy chain (γl):
Short linker:
ASTKGP;
Long linker:
ASTKGPSVFPLAP

### For DVDBA Constructs:

light chain (if anti-B has λ)
Short linker:
QPKAAP;
Long linker:
QPKAAPSVTLFPP
light chain (if anti-B has κ)
Short linker:
TVAAP;
Long linker:
TVAAPSVFIFPP
heavy chain (γl):
Short linker:
ASTKGP;
Long linker:
ASTKGPSVFPLAP

Heavy and light chain constructs are subcloned into the pBOS expression vector, and expressed in COS cells, followed by purification by TNF chromatography. The purified materials are subjected to SDS-PAGE and SEC analysis.

The Table below describes the heavy chain and light chain constructs used to express each anti-TNF/IL-33 DVD-Ig protein.

TABLE
Anti-A/B DVD-Ig Constructs

| DVD-Ig protein | Heavy chain construct | Light chain construct |
|---|---|---|
| DVDABSL | DVDABHC-SL | DVDABLC-SL |
| DVDABLL | DVDABHC-LL | DVDABLC-LL |
| DVDBASL | DVDBAHC-SL | DVDBALC-SL |
| DVDBALL | DVDBAHC-LL | DVDBALC-LL |

### Molecular Cloning of DNA Constructs for DVDABSL and DVDABLL

To generate heavy chain constructs DVDABHC-LL and DVDABHC-SL, VH domain of TNF antibody is PCR amplified using specific primers (3′ primers contain short/long linker sequence for SL/LL constructs, respectively); meanwhile VH domain of IL-33 antibody is amplified using specific primers (5′ primers contains short/long linker sequence for SL/LL constructs, respectively). Both PCR reactions are performed according to standard PCR techniques and procedures. The two PCR products are gel-purified, and used together as overlapping template for the subsequent overlapping PCR reaction. The overlapping PCR products are subcloned into Srf I and Sal I double digested pBOS-hCγl,z non-a mammalian expression vector by using standard homologous recombination approach.

To generate light chain constructs DVDABLC-LL and DVDABLC-SL, VL domain of TNF antibody is PCR amplified using specific primers (3′ primers contain short/long linker sequence for SL/LL constructs, respectively); meanwhile VL domain of IL-33 antibody is amplified using specific primers (5′ primers contains short/long linker sequence for SL/LL constructs, respectively). Both PCR reactions are performed according to standard PCR techniques and procedures. The two PCR products are gel-purified, and are used together as overlapping template for the subsequent overlapping PCR reaction using standard PCR conditions. The overlapping PCR products are subcloned into Srf I and Not I double digested pBOS-hCk mammalian expression vector (Abbott) by using standard homologous recombination approach. Similar approach has been used to generate DVDBASL and DVDBALL as described below:

### Molecular Cloning of DNA Constructs for DVDBASL and DVDBALL

To generate heavy chain constructs DVDBAHC-LL and DVDBAHC-SL, VH domain of IL-33 antibody is PCR amplified using specific primers (3′ primers contain short/long linker sequence for SL/LL constructs, respectively); meanwhile VH domain of antibody TNF is amplified using specific primers (5′ primers contains short/long linker sequence for SL/LL constructs, respectively). Both PCR reactions are performed according to standard PCR techniques and procedures. The two PCR products are gel-purified, and used together as overlapping template for the subsequent overlapping PCR reaction using standard PCR conditions. The overlapping PCR products are subcloned into Srf I and Sal I double digested pBOS-hCγl,z non-a mammalian expression vector by using standard homologous recombination approach.

To generate light chain constructs DVDBALC-LL and DVDBALC-SL, VL domain of antibody B is PCR amplified using specific primers (3′ primers contain short/long linker sequence for SL/LL constructs, respectively); meanwhile VL domain of antibody TNF is amplified using specific primers (5′ primers contains short/long linker sequence for SL/LL constructs, respectively). Both PCR reactions are performed according to standard PCR techniques and procedures. The two PCR products are gel-purified, and used together as overlapping template for the subsequent overlapping PCR reaction using standard PCR conditions. The overlapping PCR products are subcloned into Srf I and Not I double digested pBOS-hCk mammalian expression vector by using standard homologous recombination approach.

### Construction and Expression of Additional DVD-Ig

### Preparation of DVD-Ig Vector Constructs

Parent antibody amino acid sequences for specific antibodies, which recognize specific antigens or epitopes thereof, for incorporation into a DVD-Ig can be obtained by preparation of hybridomas as described above or can be obtained by sequencing known antibody proteins or nucleic acids. In addition, known sequences can be obtained from the literature. Several specific sequences related to antibodies of TNF and sequences related to antibodies of IL-33 are listed in this application. The sequences can be used to synthesize nucleic acids using standard DNA synthesis or amplification technologies and assembling the desired antibody fragments into expression vectors, using standard recombinant DNA technology, for expression in cells.

Expression of the reference antibodies and DVD-lgs is accomplished by known methods and the reference antibodies are purified by a column.

### Characterization and Lead Selection of A/B DVD-Igs

The binding affinities of anti-TNF and anti-IL-33 DVD-Igs are analyzed on Biacore against both TNF and 11-33. The tetravalent property of the DVD-Ig is examined by multiple binding studies on Biacore. Meanwhile, the neutralization potency of the DVD-Igs for TNF and Il-33 are assessed by bioassays, respectively, as described herein. The DVD-Ig molecules that best retain the affinity and potency of the original parent mAbs are selected for in-depth physicochemical and bio-analytical (rat PK) characterizations as described herein for each mAb. Based on the collection of analyses, the final lead DVD-Ig is advanced into CHO stable cell line development, and the CHO-derived material is employed in stability, pharmacokinetic and efficacy studies in cynomolgus monkey, and preformulation activities.

### Example 21: Generation and Characterization of Dual Variable Domain Immunoglobulins (DVD-Ig)

Dual variable domain immunoglobulins (DVD-Ig) using parent antibodies with known amino acid sequences are generated by synthesizing polynucleotide fragments encoding DVD-Ig variable heavy and DVD-Ig variable light chain sequences and cloning the fragments into a pHybC-D2 vector according to the preparation of DVD-Ig Vector Constructs described above. The DVD-Ig constructs are cloned into and expressed in 293 cells as described above. The DVD-Ig protein was purified according to standard methods. Functional characteristics were determined.

### Example 22

An anti-TNF and IL-33 bispecific antibody of the present invention is a combined scFv construct and is made using a dual variable antibody approach. The construction of this antibody starts with two single clain Fv (scFv) fragments (see Economides, A. N., et al. (2003) Nat. Med. 9(1): 47-52), one that recognizes the antigen TNF antigen and another that recognizes an IL-33 antigen. The two scFc fragments are linked with an additional peptide linker. The peptide linkers are used including linkers up to 63 residues. Examples of these linkers are described above.

### Example 23: IL-33/TNF Bispecific Antibody for Treating Dupuytren's Disease

Disclosed herein is the use of a TNF antagonist for the treatment of Dupuytren's disease.

It is contemplated that periodically administering a IL-33/TNF bispecific antibody to a human patient afflicted with Dupuytren's disease provides increased efficacy (provides at least an additive effect or more than an additive effect) in treating the patient than when a TNF antibody is administered alone or when an IL-33 antibody is administered alone (at the same dose). The bispecific antibody also provides efficacy (provides at least an additive effect or more than an additive effect) in treating the patient without undue adverse side effects or affecting the safety of the treatment.

The bispecific antibody provides a clinically meaningful advantage and is more effective (provides at least an additive effect or more than an additive effect) in treating the patient than when the IL-33 antibody or a TNF antibody is administered alone (at the same dose) in the following manner:

1. The bispecific antibody is effective (provides at least an additive effect or more than an additive effect) in improving nodule size and vascularity.
2. The bispecific antibody is effective (provides at least an additive effect or more than an additive effect) in improving grip strength and range of motion of the affected digit.
3. The bispecific antibody reduces the frequency of injections needed to treat Dupuytren's disease.
4. The bispecific antibody is effective in (provides at least an additive effect or more than an additive effect) treating a greater percentage of patients than either the IL-33 antagonist alone or the TNF antagonist alone.
5. The bispecific antibody delays the progression of early disease stage.

### Example 24

It is contemplated that periodically administering a therapeutically effective amount of a IL-33/TNF bispecific antibody to a patient suffering from Dupuytren's disease successfully treats such a patient.

### Example 25

It is contemplated that periodically administering a therapeutically effective amount of a IL-33/TNF bispecific antibody to a patient suffering from frozen shoulder successfully treats such a patient.

### Example 26

It is contemplated that periodically administering a therapeutically effective amount of a IL-33/TNF bispecific antibody to a patient suffering from periarticular fibrosis successfully treats the patient.

### Example 27

It is contemplated that periodically administering a therapeutically effective amount of a IL-33/TNF bispecific antibody to a patient suffering from keloid or hypertrophic scars successfully treats such a patient.

### Example 28

It is contemplated that periodically administering a therapeutically effective amount of a IL-33/TNF bispecific antibody to a patient suffering from endometriosis successfully treats such a patient.

### Example 29

It is contemplated that periodically administering a therapeutically effective amount of a IL-33/TNF bispecific antibody to a patient suffering from abdominal adhesions successfully treats such a patient.

### Example 30

It is contemplated that periodically administering a therapeutically effective amount of a IL-33/TNF bispecific antibody to a patient suffering from Ledderhose disease successfully treats such a patient.

### Example 31

It is contemplated that periodically administering a therapeutically effective amount of a IL-33/TNF bispecific antibody to a patient suffering from Peyronie's disease successfully treats such a patient.

### Example 32

It is contemplated that periodically administering a therapeutically effective amount of a IL-33/TNF bispecific antibody to a patient suffering from peritendinous adhesions successfully treats such a patient.

### Example 33

It is contemplated that periodically administering a therapeutically effective amount of a IL-33/TNF bispecific antibody to a patient suffering from breast implant capsules successfully treats such a patient.

### Example 34

It is contemplated that periodically administering a therapeutically effective amount of a IL-33/TNF bispecific antibody to a patient suffering from pelvic adhesions successfully treats such a patient.

### Example 35

It is contemplated that periodically administering a therapeutically effective amount of a IL-33/TNF bispecific antibody to a patient suffering from perineural fibrosis successfully treats such a patient.

### Example 36

It is contemplated that periodically administering a therapeutically effective amount of a IL-33/TNF bispecific antibody to a patient suffering from bone fibrosis successfully treats such a patient.

### Example 37

### Tissue Preparation

Tissue samples are obtained with informed consent from patients with Dupuytren's disease according to a protocol approved by the local Research Ethical Review Committee (REC 07/H0706/81). Dupuytren's myofibroblasts (MF-D) are isolated from α-SMA-rich nodules. Tissue samples into dissected into small pieces and digested in DMEM with type I collagenase in a concentration of 4 mg/mL for up to 3 h at 37° C. Cells were re-suspended in DMEM+5% fetal bovine serum. The freshly disaggregated cells are then used for the traction force microscopy (TFM) or subcultured up to passage 1.

### Antibody Inhibition

Cells are treated with neutralising anti-TNF (Dose range 1-10 ug/ml-MAB2101, R&D), neutralising anti-IL-33 (Dose range 1-100 ug/ml 0.04-4 ug/ml-500-P261, Peprotech) or isotype controls.

mRNA Levels

RNA is extracted from each sample using the QIAamp RNeasy Mini Kit (Qiagen) according to manufacturer's instructions. Isolated RNA is quantified using a NanoDrop ND-1000 spectrophotometer (NanoDrop Technologies). For cDNA the High-Capacity RNA-to-cDNA kit (4387406) is used. For real-time RT-PCR, Gene expression Assays are used for α-SMA (Hs00426835-g1), COL1a1 (Hs00164004-m1), TaqMan Fast Advanced Master Mix (4444557). Samples are run on the ABI 7900HT Fast Real-Time PCR System (Applied Biosystems). Expression is normalized to GAPDH and compared with the level of gene expression at baseline, which was assigned a value of 1.

### Protein Levels

### Western Blotting

Cell lysates are prepared in lysis buffer [25 mM Hepes (pH 7.0), 150 mM NaCl, and 1% Nonidet P-40], containing protease and phosphatase inhibitor mixture, and electrophoresed on 10% (wt/vol) SDS polyacrylamide gels, followed by electrotransfer of proteins onto PVDF transfer membranes. Membranes are blocked in 5% (wt/vol) BSA/TBS+0.05% Tween and incubated overnight at 4° C. with primary antibodies against α-SMA primary antibody. The bound HRP-conjugated secondary antibody are detected using enhanced chemiluminescence kit and visualized using Hyperfilm MP.

### Electrochemiluminescence

Cell lysates prepared as above are analyzed for levels of α-SMA using paired antibodies (Abnova H00000059-AP41) and detected using Meso Scale Discovery platform (Maryland, USA).

### Traction Force Microscopy

25 mm coverslips are coated with 0.5% (3-aminopropyl)trimethoxysilane (APTMS), washed in distilled water and subsequently treated with 0.5% glutaraldehyde. 18 mm coverslips are coated with 0.01% poly-L-lysine and washed in distilled water. After this, the 18 mm coverslips are coated with 0.2 μm crimson fluorescent beads (40:1000), washed in distilled water and allowed to air dry. A predetermined solution of acrylamide and bis-acrylamide is made with the exact ratio of each determining the final Young's Modulus (kPa) of the gel. Gels with stiffness in the range 6.4-25 kPa are used. 7 ul of the acrylamide, bisacrylamide solution is added to the 18 mm coverslips then the 25 mm coverslip is immediately placed on top and the polyacrylamide is allowed to solidify. Once the gel is formed the 18 mm coverslip is removed. To functionalize the gel 100 μL of 2 mg/ml freshly prepared sulfo-SANPAH (sulfosuccinimidyl 6-(4′-azido-2′-nitrophenylamino)hexanoate) is placed on the polyacrylamide gel, which is then immediately placed in a 365 nm UV crosslinker for 10 min. After washing the gel the surface is coated with 50 μL of 500 μg/ml fibronectin and incubated for 1 hour at 37° C. Freshly disaggregated myofibroblasts are cultured for 1 week and 1×10⁵ cells are seeded to the fibronectin-coated gel. Cells are cultured on the gels for up to 3 days.

The cells are maintained in a microscope chamber at 37° C. and stained using CellMask Green Plasma Membrane Stain (C37608). Images are acquired using a confocal laser microscope with a 63× objective and a 633 nm and 488 mm laser for bead and cell detection, respectively. The image is acquired in a 1054×1054 pixel frame with a pixel size of 0.1 μm and the region of interest selected is 20×20 μm. Following image acquisition, 50 μL of 0.05% trypsin-EDTA is added to the cells for 5 minutes. Once the cells have detached a further image is acquired. The data are analyzed using ImageJ Software (Java). The two images of the beads are combined using the stacking tool and the displacement of the beads between the two frames assessed using the particle image velocimetry ImageJ plugin (sites.google.com/site/qingzongtseng/piv). The corresponding traction map is determined using the FTTC traction force microscopy plugin (sites.google.com/site/qingzongtseng/tfm). The mean of the maximal traction force from 20 cells is then compared for different conditions.

### Atomic Force Microscopy

The stiffness of the freshly dissected Dupuytren's nodules is determined by atomic force microscopy.

### Example 38

It is contemplated that periodically administering a therapeutically effective amount of an IL-33 aptamer antagonist to a patient suffering from Dupuytren's disease, frozen shoulder, periarticular fibrosis, keloid or hypertrophic scars, endometriosis, abdominal adhesions, Ledderhose disease, Peyronie's disease, peritendinous adhesions, or periarticular fibrosis successfully treats such a patient.

### REFERENCES

Ball, C. et al. Systematic review of non-surgical treatments for early dupuytren's disease. BMC Musculoskelet Disord. 2016; 17: 345.
Barbie, D. A. et al. Systematic RNA interference reveals that oncogenic KRAS-driven cancers require TBK1. Nature 462, 108-112 (2009)
Beaudreuil J (2012) La maladie de Dupuytren en 2012. Revue du Rhumatisme Monographies 79, 126-132.
Betz N et al. Radiotherapy in early-stage Dupuytren's contracture. Long-term results after 13 years. Strahlenther Onkol. 2010 February; 186(2):82-90.
Bischoff S. C. (2007). Role of mast cells in allergic and non-allergic immune responses: comparison of human and murine data. Nat. Rev. Immunol. 7, 93-104.
Brod et al. (2000) Annals of Neurology, 47:127-131.
Bulstrode N W et al. The complications of Dupuytren's contracture surgery. J Hand Surg [Am]. 2005 September; 30(5):1021-5.
Chen N C, A systematic review of outcomes of fasciotomy, aponeurotomy, and collagenase treatments for Dupuytren's contracture. Hand (N Y). 2011 September; 6(3): 250-255.
Spiess et al. “Alternative molecular formats and therapeutic applications for bispecific antibodies.” Molecular Immunology, Volume 67, Issue 2, Part A, October 2015, Pages 95-106.
Chiu, Ya-Lin and Rana, Tariq M. siRNA function in RNAi: A chemical modification analysis. RNA (2003), 9:1034-1048.
Coloma, M. Josefina and Morrison, Sherie L., “Design and production of novel tetravalent bispecific antibodies.” Nature Biotechnology 15, 159-163 (1997).
Crean S M et al. The efficacy and safety of fasciectomy and fasciotomy for Dupuytren's contracture in European patients: a structured review of published studies. J Hand Surg Eur Vol. 2011 June; 36(5):396-407.
Dar, S. A. et al. siRNAmod: A database of experimentally validated chemically modified siRNAs. Sci. Rep. 6, 20031; doi: 10.1038/srep20031 (2016).
Davis et al., Gaps in exposure to essential competencies in hand surgery fellowship training: a national survey of program directors. Hand (N Y). 2013 March; 8(1): 1-11.
Deleavey, Glen F. and Damha, Masad J. Designing Chemically Modified Oligonucleotides for Targeted Gene Silencing, Chemistry & Biology, Volume 19, Issue 8, 24 Aug. 2012, Pages 937-954
National Institute for Health and Clinical Excellence (NICE), Radiation therapy for early Dupuytren's disease, NICE interventional procedure guidance 368 issued November 2010.
Feng Li. et al. Cell culture processes for monoclonal antibody production. MAbs. 2010 September-October; 2(5): 466-477.
Friedlander M, Fibrosis and diseases of the eye. J Clin Invest. 2007 Mar. 1; 117(3): 576-586.
FDA 2005. Draft Guidance for Industry—Systemic Lupus Erythematosus—Developing Drugs for Treatment.
Gaglione, M. & Messere, A. Recent progress in chemically modified siRNAs. Mini reviews in medicinal chemistry 10, 578-595 (2010).
Gailit J et al. The differentiation and function of myofibroblasts is regulated by mast cell mediators. J Invest Dermatol. 2001 November; 117(5):1113-9.
Hindocha S et al. “Epidemiological Evaluation of Dupuytren's Disease Incidence and Prevalence Rates in Relation to Etiology.” Hand (N Y). 2009 September; 4(3): 256-269.
Laganà, A et al. Computational Design of Artificial RNA Molecules for Gene Regulation. Methods Mol Biol. 2015; 1269: 393-412.
Langer-Gould et al. (2005) New England Journal of Medicine, 353:369-379.
Luo, J. et al. A genome-wide RNAi screen identifies multiple synthetic lethal interactions with the Ras oncogene. Cell 137, 835-848 (2009)
Hughes T B et al. Dupuytren's Disease. J Hand Surg Am 2003; 3:27-40.
Hurst et al. “Injectable Collagenase Clostridium Histolyticum for Dupuytren's Contracture” N ENGL J MED 361; 10 Sep. 3, 2009.
Hinz B, Alpha-Smooth Muscle Actin Expression Upregulates Fibroblast Contractile Activity. Mol Biol Cell. 2001 September; 12(9): 2730-2741.
Hinz B. Myofibroblasts. Experimental Eye Research 2016; 142:56-70.
Ishikawa K, et al. Molecular mechanisms of subretinal fibrosis in age-related macular degeneration. Experimental Eye Research 2016; 142:19-25.
Jakob, Clarissa G. et al. “Structure reveals function of the dual variable domain immunoglobulin (DVD-Ig™) molecule.” MAbs. 2013 May-June; 5(3):358-63.
Kanasty, R. et al. Delivery materials for siRNA therapeutics. Nature Mater. 12, 967-977 (2013).
Kakkar T, Lee R T, The IL-33/ST2 pathway: therapeutic target and novel biomarker. Nat Rev Drug Discov. 2008 October; 7(10):827-40.
Ketchum L D, Donahue T K, The injection of nodules of Dupuytren's disease with triamcinolone acetonide. J Hand Surg Am. 2000 November; 25(6):1157-62.
Kleinschmidt-DeMasters et al. (2005) New England Journal of Medicine, 353:369-379.
Klein C, et al. Progress in overcoming the chain association issue in bispecific heterodimeric IgG antibodies. mAbs 2012; 4(6):653-663.
Kobori A et al. (2010) Interleukin-33 expression is specifically enhanced in inflamed mucosa of ulcerative colitis. J Gastroenterol.
Kontermann, R E, Bispecific Antibodies. Springer 2011.
Kontermann R E, “Dual targeting strategies with bispecific antibodies” mAbs 4:2, 182-197; March/April 2012.
Kunisch E et al. IL-33 regulates TNF-α dependent effects in synovial fibroblasts. Int J Mol Med. 2012 April; 29(4):530-40.
Larson D et al. Clinical effectiveness of post-operative splinting after surgical release of Dupuytren's contracture: a systematic review. BMC Musculoskeletal Disorders 2008, 9:104.
Liu C et al. “Surgical correction of ptosis in ocular fibrosis Syndrome” British Journal of Ophthalmology 1994; 78: 271-274
Moulin D. (2007) Interleukin (IL)-33 induces the release of pro-inflammatory mediators by mast cells. Cytokine 40, 216-225.
Martin and Martin, Interleukin 33 is a guardian of barriers and a local alarmin. Nature Immunology; Vol 17, No. 2, February 2016.
Paddison P J, et al. Short hairpin RNAs (shRNAs) induce sequence-specific silencing in mammalian cells. Genes Dev 2002; 16:948-958.
Paladini et al. “Mutations in the Catalytic Domain of Human Matrix Metalloproteinase-l (MMP-l) That Allow for Regulated Activity through the Use of Ca2+” THE JOURNAL OF BIOLOGICAL CHEMISTRY VOL. 288, NO. 09. pp. 6629-6639, Mar. 1, 2013.
Palmer G, Gabay C, “Interleukin-33 biology with potential insights into human diseases.” Nat. Rev. Rheumatol. 7, 321-329 (2011)
Pastorelli L. et al. (2010) Epithelial-derived IL-33 and its receptor ST2 are dysregulated in ulcerative colitis and in experimental Th1/Th2 driven enteritis. Proc Natl Acad Sci USA 107: 8017-8022.
Peimer C A at al., Dupuytren contracture recurrence following treatment with collagenase clostridium histolyticum (CORDLESS study): 3-year data. J Hand Surg Am. 2013 January; 38(1):12-22.
Pohl S, et al. (2002) Prophylactic radiotherapy in Dupuytren's contracture: three years outcome. International Journal of Radiation Oncology Biology Physics 54: 23.
Rankin A L et al. IL-33 induces IL-13-dependent cutaneous fibrosis. J Immunol. 2010 Feb. 1; 184(3):1526-35.
Rayan G. M. (2007). Dupuytren disease: anatomy, pathology, presentation, and treatment. J. Bone Joint Surg. Am. 89, 189-198.
Rombouts, J Hand Surg Am, 14, 644-652, 1989.
Schiering C et al. The alarmin IL-33 promotes regulatory T-cell function in the intestine. Nature. 2014 Sep. 25; 513 (7519):564-8.
Silva, J. M. et al. Profiling essential genes in human mammary cells by multiplex RNAi screening. Science 319, 617-620 (2008)
Rao, D. D. et al. Enhanced target gene knockdown by a bifunctional shRNA: a novel approach of RNA interference. Cancer Gene Ther. 17:780-791 (2010).
Rudick, R. (1999) “Disease-Modifying Drugs for Relapsing-Remitting Multiple Sclerosis and Future Directions for Multiple Sclerosis Therapeutics”, Neurotherpatueics. 56:1079-84.
Schmitz J et al. IL-33, an interleukin-1-like cytokine that signals via the IL-1 receptor-related protein ST2 and induces T helper type 2-associated cytokines. Immunity. 2005 November; 23(5):479-90.
Seegenschmiedt M et al. “Radiotherapy for Non-Malignant Disorders” Springer (Berlin, New York, 2008).
Sponheim J et al. Inflammatory bowel disease-associated interleukin-33 is preferentially expressed in ulceration-associated myofibroblasts. Am J Pathol 2010; 177:2804-2815.
Summary basis of approval XIAFLEX-EU: Available at www.ema.europa.eu/docs/en_GB/document_library/EPAR_-_Product_Information/human/002048/WC500103373.pdf
Taylor M C (2005) Interviewing. In Qualitative Research in Healthcare (Holloway I ed). Open University Press, Berkshire, pp. 39-53.
Chang K, et al. Tools for studying and using small RNAs: from pathways to functions to therapies. Nature Reviews, Genetics Sep. 18, 2014.
Tracy et al. “Tumor necrosis factor antagonist mechanisms of action: A comprehensive review” Pharmacology & Therapeutics 117 (2008) 244-279.
Tukel et al. A new syndrome, congenital extraocular muscle fibrosis with ulnar hand anomalies, maps to chromosome 21qter. J Med Genet 2005; 42:408-415.
Ullah A S et al. (2009) Does a ‘firebreak’ full-thickness skin graft prevent recurrence after surgery for Dupuytren's contracture?: a prospective, randomised trial. J Bone Joint Surg Br 91: 374-378.
van Rijssen A L et al. Five-year results of a randomized clinical trial on treatment in Dupuytren's disease: percutaneous needle fasciotomy versus limited fasciectomy. Plast Reconstr Surg. 2012 February; 129(2):469-77.
Verjee at al. “Unraveling the signaling pathways promoting fibrosis in Dupuytren's disease reveals TNF as a therapeutic target” PNAS vol. 110 no. 10 published Feb. 19, 2013.
Vollmer et al. (2008) “Pridopidine after induction therapy with mitoxantrone in relapsing multiple sclerosis” Multiple Sclerosis, //00:1-8.
Xu D et al. IL-33 exacerbates antigen-induced arthritis by activating mast cells. PNAS, Aug. 5, 2008, vol. 105, no. 31, 10913-10918.

What is claimed is:

1. A method of treating a patient suffering from a localized fibrotic condition which comprises administering to the patient an amount of an IL-33 antagonist effective to treat the patient.

2. A method of treating a patient suffering from a localized fibrotic condition which comprises administering to the patient an amount of a bispecific antibody comprising
a) a IL-33 antigen binding domain of which (i) binds to and inhibits activation of, an IL-33 receptor, or (ii) specifically binds to IL-33 and inhibits IL-33 from binding to the IL-33 receptor, and
b) a TNF antigen binding domain of which (i) binds to and inhibits activation of, a TNF receptor, or (ii) specifically binds to TNF and inhibits TNF from binding to the TNF receptor,
wherein the bispecific antibody is effective to treat the patient.

3. The method of claim 1 or 2, wherein the localized fibrotic condition is Dupuytren's disease, frozen shoulder (adhesive capsulitis), periarticular fibrosis, scars, endometriosis, abdominal adhesions, pelvic adhesions, perineural fibrosis, Ledderhose disease, Peyronie's disease, peritendinous adhesions, peri-implant capsules, periarticular fibrosis, or bone fibrosis.

4. The method of claim 1 or 2, wherein the localized fibrotic condition is Dupuytren's disease.

5. The method of claim 1 or 2, wherein the localized fibrotic condition is early disease stage Dupuytren's disease.

6. The method of claim 1 or 2, wherein the localized fibrotic condition is frozen shoulder (adhesive capsulitis).

7. The method of claim 1 or 2, wherein the localized fibrotic condition is periarticular fibrosis.

8. The method of claim 1 or 2, wherein the localized fibrotic condition is keloid or hypertrophic scars.

9. The method of claim 1 or 2, wherein the localized fibrotic condition is Ledderhose disease.

10. The method of claim 1 or 2, wherein the localized fibrotic condition is Peyronie's disease.

11. The method of claim 1 or 2, wherein the localized fibrotic condition is endometriosis.

12. The method of claim 1 or 2, wherein the localized fibrotic condition is abdominal adhesions.

13. The method of claim 1 or 2, wherein the localized fibrotic condition is perineural fibrosis.

14. The method of claim 1 or 2, wherein the localized fibrotic condition is peritendinous adhesions.

15. The method of claim 1 or 2, wherein the localized fibrotic condition is periarticular fibrosis.

16. The method of claim 15, wherein the patient began suffering from periarticular fibrosis after an injury or after an arthroplasty.

17. The method of claim 1 or 2, wherein the localized fibrotic condition is scars.

18. The method of claim 17, wherein scars is keloid scars, hypertrophic scars, burn scars, post traumatic, or post-surgical scars.

19. The method of claim 1 or 2, wherein the localized fibrotic condition is abdominal adhesions or pelvic adhesions.

20. The method of claim 1 or 2, wherein the localized fibrotic condition is breast implant capsules or fibrosis around other implants.

21. The method of claim 1 or 2, wherein the localized fibrotic condition is bone fibrosis.

22. The method of claim 21, wherein the bone fibrosis is fibrous dysplasia.

23. The method of any one of claims 1 and 3-22, wherein the IL-33 antagonist is
a) an antibody, or antigen binding fragment of an antibody, that specifically binds to, and inhibits activation of, an IL-33 receptor;
b) a soluble form of an IL-33 receptor that specifically binds to IL-33 and inhibits IL-33 from binding to the IL-33 receptor;
c) an antisense oligonucleotide that specifically inhibits synthesis of IL-33;
d) a small molecule that specifically inhibits the activity of IL-33;
e) a bispecific antibody comprising at least one antigen binding domain of which binds to and inhibits activation of, an IL-33 receptor; or
f) small interfering RNA (siRNA) that specifically inhibits synthesis of IL-33.

24. The method of claim 23, wherein the IL-33 antagonist is a bispecific antibody and a
i) asymmetric IgG-like bispecific antibody;
ii) symmetric IgG-like bispecific antibody;
iii) IgG fusion bispecific antibody;
iv) Fc fusion bispecific antibody;
v) Fab fusion bispecific antibody;
vi) ScFv- or diabody-based bispecific antibody; or
vii) IgG/Non-IgG fusion bispecific antibody.

25. The method of any one of claims 1 and 3-24, wherein the IL-33 antagonist is an antibody which is a chimeric antibody, a humanized antibody, a human antibody, or an antigen binding fragment of a chimeric humanized and human antibody.

26. The method of claims 1 and 3-23, wherein the IL-33 antagonist is a soluble IL-1R4 polypeptide, a soluble IL-1RAP protein or ANB020.

27. The method of any one of claims 1, 3-22, and 26 wherein the IL-33 antagonist is a plasmid encoding monoclonal antibody (Mab) or a AAV encoding Mab.

28. The method of any one of claims 1 and 3-22, wherein the IL-33 antagonist is a RNA interference (RNAi) antagonist.

29. The method of claim 28, wherein the RNAi antagonist is a AAV-RNAi.

30. The method of any one of claims 1, 3-22, and 28-29 wherein the IL-33 antagonist is:
a) a small interfering RNA (siRNA);
b) a short hairpin RNA (shRNA); or
c) a siRNA that specifically inhibits synthesis of IL-33.

31. The method of any one of claims 28-30, wherein the RNAi antagonist, the siRNA or the shRNA is directed to and targeting the IL-33 receptor IL-1R4.

32. The method of any one of claims 1 and 3-22, wherein the IL-33 antagonist is an aptamer antagonist.

33. The method of any one of claims 2-24, wherein the bispecific antibody is a
i) asymmetric IgG-like bispecific antibody,
ii) symmetric IgG-like bispecific antibody,
iii) IgG fusion bispecific antibody,
iv) Fc fusion bispecific antibody,
v) Fab fusion bispecific antibody,
vi) ScFv- or diabody-based bispecific antibody,
vii) IgG/Non-IgG fusion bispecific antibody, or
viii) fragment-based bispecific antibody.

34. The method of any one of claims 2-24 and 33, wherein the bispecific antibody is a bispecific monoclonal antibody inhibitor.

35. The method of any one of claims 2-24 and 33, wherein the bispecific antibody is a viral vector.

36. The method of any one of claims 2-24 and 33, wherein the bispecific antibody is expressed in an adeno-associated virus (AAV) expression vector.

37. The method of any one of claims 2-24 and 33-36, wherein the bispecific antibody is a combined single-chain variable fragment (scFv) construct.

38. The method of any one of claims 2-24 and 33-37, wherein the bispecific antibody is made by a dual variable antibody approach.

39. The method of any one of claims 2-24 and 33-38, wherein the bispecific antibody further comprises a transcriptional promotor that is expressed only in myofibroblasts.

40. The method of any one of claims 2-24 and 33-39, wherein the IL-33 antigen binding domain is a direct IL-33 antagonist.

41. The method of any one of claims 2-24 and 33-39, wherein the IL-33 antigen binding domain is an anti-IL-1R4 receptor antagonist.

42. The method of any one of claims 2-24 and 33-39, wherein the IL-33 antigen binding domain is the binding domain of an antibody, wherein the antibody is a chimeric antibody, a humanized antibody, a human antibody, or an antigen binding fragment of a chimeric humanized and human antibody.

43. The method of any one of claims 2-24 and 33-40, wherein the IL-33 antigen binding domain is from the binding domains of a soluble IL-1R4 polypeptide, a soluble IL-1RAP protein or ANB020.

44. The method of any one of claims 2-24 and 33-43, wherein the IL-33 antigen binding domain specifically targets the IL-33 receptor IL-1R4.

45. The method of any one of claims 2-24 and 33-39, wherein the IL-33 antigen binding domain (a) binds to the cytokine IL-33, preferably neutralizing biological function, (b) is an antibody to the cytokine IL-33, (c) is an antibody to IR-1R4, (d) is an antibody to IR-1R3 (e) is a IL-1R4 soluble receptor, or (f) is a IL-1R3 soluble receptor.

46. The method of any one of claims 1-45, wherein the IL-33 antagonist or the bispecific antibody is administered orally, intralesionally, by intravenous therapy or by subcutaneous, intramuscular, intraarterial, intravenous, intracavitary, intracranial, or intraperitoneal injection.

47. The method of claim 46, wherein the IL-33 antagonist or bispecific antibody is administered by intravenous injection.

48. The method of claim 46, wherein the IL-33 antagonist or bispecific antibody is administered orally.

49. The method of claim 46, wherein the IL-33 antagonist or bispecific antibody is injected directly into the affected tissue.

50. The method of claim 46, wherein the IL-33 antagonist or bispecific antibody is injected to a site of maximal cellularity or maximal inflammation.

51. The method of any one of claims 1-50, wherein the IL-33 antagonist or bispecific antibody is administered daily.

52. The method of any one of claims 1-50, wherein the IL-33 antagonist or bispecific antibody is administered weekly.

53. The method of any one of claims 1-50, wherein the IL-33 antagonist or bispecific antibody is administered monthly.

54. The method of any one of claims 1-50, wherein the IL-33 antagonist or bispecific antibody is administered biweekly, once every two months, once every three months, once every 6 months, or once every 12 months.

55. The method of any one of claims 1, 3-32, and 46-54 wherein the effective amount of the IL-33 antagonist is an amount between about 0.1 mg and about 500 mg.

56. A method of any one of claims 1, 3-32, and 46-55, which further comprises co-administering a TNF antagonist.

57. The method of claim 56, wherein the administration of the IL-33 antagonist precedes the administration of the TNF antagonist.

58. The method of claim 56, wherein the patient is receiving the IL-33 antagonist prior to initiating administering the TNF antagonist and continues to receive the IL-33 antagonist after administration of the TNF antagonist is initiated.

59. The method of claim 56, wherein the administration of the TNF antagonist precedes the administration of the IL-33 antagonist.

60. The method of claim 56, wherein the patient is receiving the TNF antagonist prior to initiating administering the IL-33 antagonist and continues to receive the IL-33 antagonist after administration of the TNF antagonist is initiated.

61. The method of any one of claims 56-60, wherein the TNF antagonist is administered in an amount between about 0.05 and about 5.0 times the clinical dose of the TNF antagonist typically administered to a patient with rheumatoid arthritis.

62. The method any one of claims 56-61, wherein the amount of the TNF antagonist is between about 5 mg and about 300 mg.

63. The method of any one of claims 56-62, wherein the TNF antagonist is one or more of infliximab, adalimumab, certolizumab pegol, golimumab or etanercept.

64. The method of claim 63, wherein the TNF antagonist is golimumab and the amount of golimumab administered is between about 1 mg and about 90 mg.

65. The method of claim 63, wherein the TNF antagonist is adalimumab and the amount of adalimumab administered is between about 5 mg and about 100 mg.

66. The method of claim 63, wherein the TNF antagonist is certolizumab pegol and the amount of certolizumab pegol administered is between about 50 mg and about 200 mg.

67. The method of claim 63, wherein the TNF antagonist is infliximab and the amount of infliximab administered is between about 50 mg and about 300 mg.

68. The method of claim 63, wherein the TNF antagonist is etanercept and the amount of etanercept administered is between about 5 mg and about 50 mg.

69. The method of any one of claims 56-60, wherein the TNF antagonist is an aptamer antagonist.

70. The method of any one of claims 56-62 and 69, wherein the TNF antagonist is a TNF receptor 1 (TNFR1) antagonist.

71. The method of any one of claims 56-62 and 69-70, wherein the TNF antagonist is a TNF receptor 2 (TNFR2) antagonist.

72. The method of any one of claims 56-62 and 70-71, wherein the TNF antagonist is an antisense oligonucleotide.

73. The method of any one of claims 56-62 and 70-71, wherein the TNF antagonist is a RNA interference (RNAi) antagonist.

74. The method of claim 73, wherein the RNAi antagonist is an AAV-RNAi.

75. The method of any one of claims 56-62 and 70-71, wherein the TNF antagonist is a plasmid encoding Mab or an AAV encoding Mab.

76. The method of any one of claims 56-62 and 70-71, wherein the TNF antagonist is:
a) a siRNA; or
b) a shRNA.

77. A method of any one of claims 1, 3-32, and 46-76, which further comprises co-administering a GM-CSF antagonist.

78. The method of claim 77, wherein the administration of the IL-33 antagonist precedes the administration of the GM-CSF antagonist.

79. The method of claim 77, wherein the patient is receiving the IL-33 antagonist prior to initiating administering the GM-CSF antagonist and continues to receive the IL-33 antagonist after administration of the GM-CSF antagonist is initiated.

80. The method of claim 77, wherein the administration of the GM-CSF antagonist precedes the administration of the IL-33 antagonist.

81. The method of claim 77, wherein the patient is receiving the GM-CSF antagonist prior to initiating administering the IL-33 antagonist and continues to receive the IL-33 antagonist after administration of the GM-CSF antagonist is initiated.

82. A method of any one of claims 1, 3-32, and 46-81, which further comprises co-administering one or more of an IL-17 antagonist, an IL-21 antagonist or an IL-23 antagonist.

83. The method of claim 82, wherein the administration of the IL-33 antagonist precedes the administration of the one or more of the IL-17 antagonist, the IL-21 antagonist, or the IL-23 antagonist.

84. The method of claim 82, wherein the patient is receiving the IL-33 antagonist prior to initiating administering the one or more of the IL-17 antagonist, the IL-21 antagonist, or the IL-23 antagonist and continues to receive the IL-33 antagonist after administration of the one or more of the IL-17 antagonist, the IL-21 antagonist, or the IL-23 antagonist is initiated.

85. The method of claim 82, wherein the administration of the one or more of the IL-17 antagonist, the IL-21 antagonist, or the IL-23 antagonist precedes the administration of the IL-33 antagonist.

86. The method of claim 82, wherein the patient is receiving the one or more of the IL-17 antagonist, the IL-21 antagonist, or the IL-23 antagonist prior to initiating administering the IL-33 antagonist and continues to receive the IL-33 antagonist after administration of the one or more of the IL-17 antagonist, the IL-21 antagonist, or the IL-23 antagonist is initiated.

87. The method of any one of claims 82-86, wherein the amount of the one or more of the IL-17 antagonist, the IL-21 antagonist, or the IL-23 antagonist is between about 75 mg and about 300 mg.

88. A method of any one of claims 1, 3-32, and 46-87, further comprising administering a therapeutically, prophylactically or progression-inhibiting amount of a DAMP antagonist and/or an AGE inhibitor to the patient.

89. The method of claim 88, wherein a DAMP antagonist is administered and the DAMP antagonist is an Alarmin antagonist.

90. The method of claim 88, wherein the Alarmin antagonist is one or more of an antagonist of HMGB1, an antagonist of S100A8, an antagonist of S100A9, an antagonist of SI00A8/9, and a heat shock protein.

91. The method of any one of claims 2-24 and 33-54, wherein the effective amount of the bispecific antibody is an amount between about 0.1 mg and about 500 mg.

92. The method of any one of claims 2-24, 33-54, and 90, wherein the amount of the bispecific antibody is between about 0.1 mg and about 100 mg.

93. The method of any one of claims 2-24 and 33-54, wherein the bispecific antibody is administered in an amount such that the amount of the TNF antigen binding domain is between about 0.05 and about 5.0 times the clinical dose of the TNF antigen binding domain typically administered to a patient with rheumatoid arthritis.

94. The method of any one of claims 2-24, 33-54, and 90-92 wherein the TNF antigen binding domain is an infliximab construct, adalimumab construct, certolizumab pegol construct, golimumab construct or etanercept construct.

95. The method of claim 94, wherein the TNF antigen binding domain is an adalimumab construct.

96. The method of any one of claims 2-24, 33-54, and 91-95, wherein the TNF receptor is a TNF receptor 1 (TNFR1) and a TNF receptor 2 (TNFR2).

97. The method of any one of claims 2-24, 33-54, and 91-95, wherein the TNF receptor is a TNFR1.

98. The method of any one of claims 2-24, 33-54, and 91-95, wherein the TNF receptor is a TNFR2.

99. The method of any one of claims 2-24, 33-54, and 91-95, wherein the TNF antigen binding domain binds to and inhibits TNF from binding to TNFR1 and TNFR2.

100. The method of any one of claims 2-24, 33-54, and 91-95, wherein the TNF antigen binding domain binds to and inhibits TNF from binding to TNFR1.

101. The method of any one of claims 2-24, 33-54, and 91-95, wherein the TNF antigen binding domain binds to and inhibits TNF from binding to TNFR2.


SEQUENCE LISTING (39 sequences)

SEQ ID NO: 1 - DNA, 21 bp, Homo sapiens
gccuugggucuacuaauaatt

SEQ ID NO: 2 - DNA, 21 bp, Homo sapiens
cggugacugucccaacuuutt

SEQ ID NO: 3 - DNA, 21 bp, Homo sapiens
guuacaccguggauugguatt

SEQ ID NO: 4 - DNA, 2644 bp, Homo sapiens
caacagaatactgaaaaatgaagcctaaaatgaagtattcaaccaacaaaatttccacagcaaagtggaagaacacagcaagcaaagccttgtgtttcaagctgggaaaatcccaacagaaggccaaagaagtttgccccatgtactttatgaagctccgctctggccttatgataaaaaaggaggcctgttactttaggagagaaaccaccaaaaggccttcactgaaaacaggtagaaagcacaaaagacatctggtactcgctgcctgtcaacagcagtctactgtggagtgctttgcctttggtatatcaggggtccagaaatatactagagcacttcatgattcaagtatcacaggaatttcacctattacagagtatcttgcttctctaagcacatacaatgatcaatccattacttttgctttggaggatgaaagttatgagatatatgttgaagacttgaaaaaagatgaaaagaaagataaggtgttactgagttactatgagtctcaacacccctcaaatgaatcaggtgacggtgttgatggtaagatgttaatggtaaccctgagtcctacaaaagacttctggttgcatgccaacaacaaggaacactctgtggagctccataagtgtgaaaaaccactgccagaccaggccttctttgtccttcataatatgcactccaactgtgtttcatttgaatgcaagactgatcctggagtgtttataggtgtaaaggataatcatcttgctctgattaaagtagactcttctgagaatttgtgtactgaaaatatcttgtttaagctctctgaaacttagttgatggaaacctgtgagtcttgggttgagtacccaaatgctaccactggagaaggaatgagagataaagaaagagacaggtgacatctaagggaaatgaagagtgcttagcatgtgtggaatgttttccatattatgtataaaaatattttttctaatcctccagttattcttttatttccctctgtataactgcatcttcaatacaagtatcagtatattaaatagggtattggtaaagaaacggtcaacattctaaagagatacagtctgacctttacttttctctagtttcagtccagaaagaacttcatatttagagctaaggccactgaggaaagagccatagcttaagtctctatgtagacagggatccattttaaagagctacttagagaaataattttccacagttccaaacgataggctcaaacactagagctgctagtaaaaagaagaccagatgcttcacagaattatcattttttcaactggaataaaacaccaggtttgtttgtagatgtcttaggcaacactcagagcagatctcccttactgtcaggggatatggaacttcaaaggcccacatggcaagccaggtaacataaatgtgtgaaaaagtaaagataactaaaaaatttagaaaaataaatccagtatttgtaaagtgaataacttcatttctaattgtttaatttttaaaattctgatttttatatattgagtttaagcaaggcattcttacacgaggaagtgaagtaaattttagttcagacataaaatttcacttattaggaatatgtaacatgctaaaacttttttttttttaaagagtactgagtcacaacatgttttagagcatccaagtaccatataatccaactatcatggtaaggccagaaatcttctaacctaccagagcctagatgagacaccgaattaacattaaaatttcagtaactgactgtccctcatgtccatggcctaccatcccttctgaccctggcttccagggacctatgtcttttaatactcactgtcacattgggcaaagttgcttctaatccttatttcccatgtgcacaagtctttttgtattccagcttcctgataacactgcttactgtggaatattcatttgacatctgtctcttttcatttcttttaactaccatgcccttgatatatcttttgcacctgctgaacttcatttctgtatcacctgacctctggatgccaaaacgtttattctgctttgtctgttgtagaattttagataaagctattaatggcaatatttttttgctaaacgtttttgttttttactgtcactagggcaataaaatttatactcaaccatataataacattttttaactactaaaggagtagtttttattttaaagtcttagcaatttctattacaacttttcttagacttaacacttatgataaatgactaacatagtaacagaatctttatgaaatatgaccttttctgaaaatacatacttttacatttctactttattgagacctattagatgtaagtgctagtagaatataagataaaagaggctgagaattaccatacaagggtattacaactgtaaaacaatttatctttgtttcattgttctgtcaataattgttaccaaagagataaaaataaaagcagaatgtatatcatcccatctgaaaaacactaattattgacatgtgcatctgtacaataaacttaaaatgattattaaataatcaaatatatctactacattgtttatattattgaataaagtatattttccaaatgt

SEQ ID NO: 5 - protein, 328 aa, Homo sapiens
  SIGNAL (1)..(18)
  CHAIN (19)..(328)
MGFWILAILTILMYSTAAKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPSKECF

SEQ ID NO: 6 - protein, 270 aa, Homo sapiens
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 7 - protein, 117 aa, Artificial Sequence
  Description of Artificial Sequence Synthetic polypeptide
EVQLQESGPGLVKPSETLSLTCTVSGFSLSDYGVNWVRQPPGKGLEWLGMIWGDGSTDYDSTLKSRLTISKDNSKSQISLKLSSVTAADTAVYYCAREWHHGPVAYWGQGTLVTVSS

SEQ ID NO: 8 - protein, 117 aa, Artificial Sequence
  Description of Artificial Sequence Synthetic polypeptide
EVQLQESGPGLVKPSETLSLTCTVSGFSLSDYGVNWIRQPPGKGLEWIGMIWGDGSTDYDSTLKSRVTISKDTSKNQFSLKLSSVTAADTAVYYCAREWHHGPVAYWGQGTLVTVSS

SEQ ID NO: 9 - protein, 117 aa, Artificial Sequence
  Description of Artificial Sequence Synthetic polypeptide
EVQLVESGGGLVQPGGSLRLSCAASGFTFSDYGVNWVRQAPGKGLEWVSMIWGDGSTDYDSTLKSRFTISRDNSKNTLYLQMNSLRAEDTAVYYCAREWHHGPVAYWGQGTLVTVSS

SEQ ID NO: 10 - protein, 117 aa, Artificial Sequence
  Description of Artificial Sequence Synthetic polypeptide
EVQLVESGGGLVQPGGSLRLSCAVSGFTLSDYGVNWVRQAPGKGLEWLGMIWGDGSTDYDSTLKSRLTISKDNSKSTIYLQMNSLRAEDTAVYYCAREWHHGPVAYWGQGTLVTVSS

SEQ ID NO: 11 - protein, 117 aa, Artificial Sequence
  Description of Artificial Sequence Synthetic polypeptide
EVQLVESGGGLVQPGGSLRLSCAASGFTLSDYGVNWVRQAPGKGLEWVSMIWGDGSTDYDSTLKSRFTISKDNSKNTLYLQMNSLRAEDTAVYYCAREWHHGPVAYWGQGTLVTVSS

SEQ ID NO: 12 - protein, 107 aa, Artificial Sequence
  Description of Artificial Sequence Synthetic polypeptide
DIQMTQSPSSLSASVGDRVTITCKASQAVSSAVAWYQQKPGKSPKLLIYWASTRHTGVPSRFSGSGSGTDFTLTISSLQPEDFATYYCQQHYSTPFTFGQGTKLEIK

SEQ ID NO: 13 - protein, 107 aa, Artificial Sequence
  Description of Artificial Sequence Synthetic polypeptide
EIVMTQSPATLSLSPGERATLSCKASQAVSSAVAWYQQKPGQAPRLLIYWASTRHTGIPARFSGSGSGTDFTLTISSLQPEDFAVYYCQQHYSTPFTFGQGTKLEIK

SEQ ID NO: 14 - protein, 107 aa, Artificial Sequence
  Description of Artificial Sequence Synthetic polypeptide
EIVMTQSPATLSLSPGERATLSCKASQAVSSAVAWYQQKPGQSPRLLIYWASTRHTGVPARFSGSGSGTDFTLTISSLQPEDFAVYYCQQHYSTPFTFGQGTKLEIK

SEQ ID NO: 15 - protein, 117 aa, Mus sp.
QVQLKESGPGLVAPSQSLSITCTVSGFSLTDYGVNWVRQPPGKGLEWLGMIWGDGSTDYDSTLKSRLSISKDNSKSQIFLKMNSLQTDDTARYYCAREWHHGPVAYWGQGTLVTVSA

SEQ ID NO: 16 - protein, 108 aa, Mus sp.
DIVMTQSHKFMSTTVGDRVSITCKASQAVSSAVAWYQQKPGQSPKLLIYWASTRHTGVPDRFTGSGSVTDFTLTIHNLQAEDLALYYCQQHYSTPFTFGSGTKLEIKR

SEQ ID NO: 17 - protein, 117 aa, Artificial Sequence
  Description of Artificial Sequence Synthetic polypeptide
QVQLQESGPGLVKPSETLSLTCTVSGGSISDYGVNWIRQPPGKGLEWIGMIWGDGSTDYDSTLKSRVTISVDTSKNQFSLKLSSVTAADTAVYYCAREWHHGPVAYWCQGTLVTVSS

SEQ ID NO: 18 - protein, 117 aa, Artificial Sequence
  Description of Artificial Sequence Synthetic polypeptide
EVQLVESGGGLIQPGGSLRLSCAASGFTVSDYGVNWVRQAPGKGLEWVSMIWGDGSTDYDSTLKSRFTISRDNSKNTLYLQMNSLRAEDTAVYYCAREWHHGPVAYWGQGTLVTVSS

SEQ ID NO: 19 - protein, 107 aa, Artificial Sequence
  Description of Artificial Sequence Synthetic polypeptide
DIQMTQSPSSLSASVGDRVTITCKASQAVSSAVAWYQQKPGKAPKLLIYWASTRHTGVPSRFSGSGSGTDFTLTISSLQPEDFATYYCQQHYSTPFTFGQGTKLEIK

SEQ ID NO: 20 - protein, 107 aa, Artificial Sequence
  Description of Artificial Sequence Synthetic polypeptide
EIVMTQSPATLSVSPGERATLSCKASQAVSSAVAWYQQKPGQAPRLLIYWASTRHTGIPARFSGSGSGTEFTLTISSLQSEDFAVYYCQQHYSTPFTFGQGTKLEIK

SEQ ID NO: 21 - protein, 117 aa, Artificial Sequence
  Description of Artificial Sequence Synthetic polypeptide
EVQLQESGPGLVKPSETLSLTCTVSGGSISDYGVNWIRQPPGKGLEWIGMIWGDGSTDYDSTLKSRVTISVDTSKNQFSLKLSSVTAADTAVYYCAREWHHGPVAYWGQGTLVTVSS

SEQ ID NO: 22 - protein, 570 aa, Artificial Sequence
  Description of Artificial Sequence Synthetic polypeptide
MIDRQRMGLWALAILTLPMYLTVTEGSKSSWGLENEALIVRCPQRGRSTYPVEWYYSDTNESIPTQKRNRIFVSRDRLKFLPARVEDSGIYACVIRSPNLNKTGYLNVTIHKKPPSCNIPDYLMYSTVRGSDKNFKITCPTIDLYNWTAPVQWFKNCKALQEPRFRAHRSYLFIDNVTHDDEGDYTCQFTHAENGTNYIVTATRSFTVEEKGFSMFPVITNPPYNHTMEVEIGKPASIACSACFGKGSHFLADVLWQINKTVVGNFGEARIQEEEGRNESSSNDMDCLTSVLRITGVTEKDLSLEYDCLALNLHGMIRHTIRLRRKQPIDHRSIYYVDPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 23 - protein, 44 aa, Homo sapiens
KKDKVLLSYYESQHPSNESGDGVDGKMLMVTLSPTKDFWLHANN

SEQ ID NO: 24 - protein, 24 aa, Homo sapiens
ESQHPSNESGDGVDGKMLMVTLSP

SEQ ID NO: 25 - protein, 4 aa, Homo sapiens
DGVD

SEQ ID NO: 26 - protein, 270 aa, Homo sapiens
MKPKMKYSTNKISTAKWKNTASKALCFKLGKSQQKAKGVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 27 - protein, 104 aa, Homo sapiens
KFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTV

SEQ ID NO: 28 - protein, 6 aa, Mus musculus
SDYAWN

SEQ ID NO: 29 - protein, 16 aa, Mus musculus
FISYSGDTSFNPSLKS

SEQ ID NO: 30 - protein, 8 aa, Mus musculus
YDGYSFDY

SEQ ID NO: 31 - protein, 15 aa, Mus musculus
RASKSVSTSGSSYMF

SEQ ID NO: 32 - protein, 7 aa, Mus musculus
LASNLES

SEQ ID NO: 33 - protein, 9 aa, Mus musculus
QHSREIPYT

SEQ ID NO: 34 - protein, 5 aa, Mus musculus
DDYMH

SEQ ID NO: 35 - protein, 17 aa, Mus musculus
RIDPAIGNTEYAPKFQD

SEQ ID NO: 36 - protein, 8 aa, Mus musculus
GDFYAMDY

SEQ ID NO: 37 - protein, 11 aa, Mus musculus
ITNTDIDDVIH

SEQ ID NO: 38 - protein, 7 aa, Mus musculus
EGNTLRP

SEQ ID NO: 39 - protein, 8 aa, Mus musculus
LQSDNMLT